SEQUENCE LISTING (18 sequences)

SEQ ID NO: 1 - protein, 2321 aa, Homo sapiens
MGPGARGRRRRRRPMSPPPPPPPVRALPLLLLLAGPGAAAPPCLDGSPCANGGRCTQLPSREAACLCPPGWVGERCQLEDPCHSGPCAGRGVCQSSVVAGTARFSCRCPRGFRGPDCSLPDPCLSSPCAHGARCSVGPDGRFLCSCPPGYQGRSCRSDVDECRVGEPCRHGGTCLNTPGSFRCQCPAGYTGPLCENPAVPCAPSPCRNGGTCRQSGDLTYDCACLPGFEGQNCEVNVDDCPGHRCLNGGTCVDGVNTYNCQCPPEWTGQFCTEDVDECQLQPNACHNGGTCFNTLGGHSCVCVNGWTGESCSQNIDDCATAVCFHGATCHDRVASFYCACPMGKTGLLCHLDDACVSNPCHEDAICDTNPVNGRAICTCPPGFTGGACDQDVDECSIGANPCEHLGRCVNTQGSFLCQCGRGYTGPRCETDVNECLSGPCRNQATCLDRIGQFTCICMAGFTGTYCEVDIDECQSSPCVNGGVCKDRVNGFSCTCPSGFSGSTCQLDVDECASTPCRNGAKCVDQPDGYECRCAEGFEGTLCDRNVDDCSPDPCHHGRCVDGIASFSCACAPGYTGTRCESQVDECRSQPCRHGGKCLDLVDKYLCRCPSGTTGVNCEVNIDDCASNPCTFGVCRDGINRYDCVCQPGFTGPLCNVEINECASSPCGEGGSCVDGENGFRCLCPPGSLPPLCLPPSHPCAHEPCSHGICYDAPGGFRCVCEPGWSGPRCSQSLARDACESQPCRAGGTCSSDGMGFHCTCPPGVQGRQCELLSPCTPNPCEHGGRCESAPGQLPVCSCPQGWQGPRCQQDVDECAGPAPCGPHGICTNLAGSFSCTCHGGYTGPSCDQDINDCDPNPCLNGGSCQDGVGSFSCSCLPGFAGPRCARDVDECLSNPCGPGTCTDHVASFTCTCPPGYGGFHCEQDLPDCSPSSCFNGGTCVDGVNSFSCLCRPGYTGAHCQHEADPCLSRPCLHGGVCSAAHPGFRCTCLESFTGPQCQTLVDWCSRQPCQNGGRCVQTGAYCLCPPGWSGRLCDIRSLPCREAAAQIGVRLEQLCQAGGQCVDEDSSHYCVCPEGRTGSHCEQEVDPCLAQPCQHGGTCRGYMGGYMCECLPGYNGDNCEDDVDECASQPCQHGGSCIDLVARYLCSCPPGTLGVLCEINEDDCGPGPPLDSGPRCLHNGTCVDLVGGFRCTCPPGYTGLRCEADINECRSGACHAAHTRDCLQDPGGGFRCLCHAGFSGPRCQTVLSPCESQPCQHGGQCRPSPGPGGGLTFTCHCAQPFWGPRCERVARSCRELQCPVGVPCQQTPRGPRCACPPGLSGPSCRSFPGSPPGASNASCAAAPCLHGGSCRPAPLAPFFRCACAQGWTGPRCEAPAAAPEVSEEPRCPRAACQAKRGDQRCDRECNSPGCGWDGGDCSLSVGDPWRQCEALQCWRLFNNSRCDPACSSPACLYDNFDCHAGGRERTCNPVYEKYCADHFADGRCDQGCNTEECGWDGLDCASEVPALLARGVLVLTVLLPPEELLRSSADFLQRLSAILRTSLRFRLDAHGQAMVFPYHRPSPGSEPRARRELAPEVIGSVVMLEIDNRLCLQSPENDHCFPDAQSAADYLGALSAVERLDFPYPLRDVRGEPLEPPEPSVPLLPLLVAGAVLLLVILVLGVMVARRKREHSTLWFPEGFSLHKDVASGHKGRREPVGQDALGMKNMAKGESLMGEVATDWMDTECPEAKRLKVEEPGMGAEEAVDCRQWTQHHLVAADIRVAPAMALTPPQGDADADGMDVNVRGPDGFTPLMLASFCGGALEPMPTEEDEADDTSASIISDLICQGAQLGARTDRTGETALHLAARYARADAAKRLLDAGADTNAQDHSGRTPLHTAVTADAQGVFQILIRNRSTDLDARMADGSTALILAARLAVEGMVEELIASHADVNAVDELGKSALHWAAAVNNVEATLALLKNGANKDMQDSKEETPLFLAAREGSYEAAKLLLDHFANREITDHLDRLPRDVAQERLHQDIVRLLDQPSGPRSPPGPHGLGPLLCPPGAFLPGLKAAQSGSKKSRRPPGKAGLGPQGPRGRGKKLTLACPGPLADSSVTLSPVDSLDSPRPFGGPPASPGGFPLEGPYAAATATAVSLAQLGGPGRAGLGRQPPGGCVLSLGLLNPVAVPLDWARLPPPAPPGPSFLLPLAPGPQLLNPGTPVSPQERPPPYLAVPGHGEEYPAAGAHSSPPKARFLRVPSEHPYLTPSPESPEHWASPSPPSLSDWSESTPSPATATGAMATTTGALPAQPLPLSVPSSLAQAQTQLGPQPEVTPKRQVLA

SEQ ID NO: 2 - DNA, 8091 bp, Homo sapiens
  source 1..8091: mol_type = genomic DNA
acgcggcgcggaggctggcccgggacgcgcccggagcccagggaaggagggaggaggggagggtcgcggccggccgccatggggccgggggcccgtggccgccgccgccgccgtcgcccgatgtcgccgccaccgccaccgccacccgtgcgggcgctgcccctgctgctgctgctagcggggccgggggctgcagcccccccttgcctggacggaagcccgtgtgcaaatggaggtcgttgcacccagctgccctcccgggaggctgcctgcctgtgcccgcctggctgggtgggtgagcggtgtcagctggaggacccctgtcactcaggcccctgtgctggccgtggtgtctgccagagttcagtggtggctggcaccgcccgattctcatgccggtgcccccgtggcttccgaggccctgactgctccctgccagatccctgcctcagcagcccttgtgcccacggtgcccgctgctcagtggggcccgatggacgcttcctctgctcctgcccacctggctaccagggccgcagctgccgaagcgacgtggatgagtgccgggtgggtgagccctgccgccatggtggcacctgcctcaacacacctggctccttccgctgccagtgtccagctggctacacagggccactatgtgagaaccccgcggtgccctgtgcgccctcaccatgccgtaacgggggcacctgcaggcagagtggcgacctcacttacgactgtgcctgtcttcctgggtttgagggtcagaattgtgaagtgaacgtggacgactgtccaggacaccgatgtctcaatggggggacatgcgtggatggcgtcaacacctataactgccagtgccctcctgagtggacaggccagttctgcacggaggacgtggatgagtgtcagctgcagcccaacgcctgccacaatgggggtacctgcttcaacacgctgggtggccacagctgcgtgtgtgtcaatggctggacaggtgagagctgcagtcagaatatcgatgactgtgccacagccgtgtgcttccatggggccacctgccatgaccgcgtggcttctttctactgtgcctgccccatgggcaagactggcctcctgtgtcacctggatgacgcctgtgtcagcaacccctgccacgaggatgctatctgtgacacaaatccggtgaacggccgggccatttgcacctgtcctcccggcttcacgggtggggcatgtgaccaggatgtggacgagtgctctatcggcgccaacccctgcgagcacttgggcaggtgcgtgaacacgcagggctccttcctgtgccagtgcggtcgtggctacactggacctcgctgtgagaccgatgtcaacgagtgtctgtcggggccctgccgaaaccaggccacgtgcctcgaccgcataggccagttcacctgtatctgtatggcaggcttcacaggaacctattgcgaggtggacattgacgagtgtcagagtagcccctgtgtcaacggtggggtctgcaaggaccgagtcaatggcttcagctgcacctgcccctcgggcttcagcggctccacgtgtcagctggacgtggacgaatgcgccagcacgccctgcaggaatggcgccaaatgcgtggaccagcccgatggctacgagtgccgctgtgccgagggctttgagggcacgctgtgtgatcgcaacgtggacgactgctcccctgacccatgccaccatggtcgctgcgtggatggcatcgccagcttctcatgtgcctgtgctcctggctacacgggcacacgctgcgagagccaggtggacgaatgccgcagccagccctgccgccatggcggcaaatgcctagacctggtggacaagtacctctgccgctgcccttctgggaccacaggtgtgaactgcgaagtgaacattgacgactgtgccagcaacccctgcacctttggagtctgccgtgatggcatcaaccgctacgactgtgtctgccaacctggcttcacagggcccctttgtaacgtggagatcaatgagtgtgcttccagcccatgcggcgagggaggttcctgtgtggatggggaaaatggcttccgctgcctctgcccgcctggctccttgcccccactctgcctccccccgagccatccctgtgcccatgagccctgcagtcacggcatctgctatgatgcacctggcgggttccgctgtgtgtgtgagcctggctggagtggcccccgctgcagccagagcctggcccgagacgcctgtgagtcccagccgtgcagggccggtgggacatgcagcagcgatggaatgggtttccactgcacctgcccgcctggtgtccagggacgtcagtgtgaactcctctccccctgcaccccgaacccctgtgagcatgggggccgctgcgagtctgcccctggccagctgcctgtctgctcctgcccccagggctggcaaggcccacgatgccagcaggatgtggacgagtgtgctggccccgcaccctgtggccctcatggtatctgcaccaacctggcagggagtttcagctgcacctgccatggagggtacactggcccttcctgtgatcaggacatcaatgactgtgaccccaacccatgcctgaacggtggctcgtgccaagacggcgtgggctccttttcctgctcctgcctccctggtttcgccggcccacgatgcgcccgcgatgtggatgagtgcctgagcaacccctgcggcccgggcacctgtaccgaccacgtggcctccttcacctgcacctgcccgccgggctacggaggcttccactgcgaacaggacctgcccgactgcagccccagctcctgcttcaatggcgggacctgtgtggacggcgtgaactcgttcagctgcctgtgccgtcccggctacacaggagcccactgccaacatgaggcagacccctgcctctcgcggccctgcctacacgggggcgtctgcagcgccgcccaccctggcttccgctgcacctgcctcgagagcttcacgggcccgcagtgccagacgctggtggattggtgcagccgccagccttgtcaaaacgggggtcgctgcgtccagactggggcctattgcctttgtccccctggatggagcggacgcctctgtgacatccgaagcttgccctgcagggaggccgcagcccagatcggggtgcggctggagcagctgtgtcaggcgggtgggcagtgtgtggatgaagacagctcccactactgcgtgtgcccagagggccgtactggtagccactgtgagcaggaggtggacccctgcttggcccagccctgccagcatggggggacctgccgtggctatatggggggctacatgtgtgagtgtcttcctggctacaatggtgataactgtgaggacgacgtggacgagtgtgcctcccagccctgccagcacgggggttcatgcattgacctcgtggcccgctatctctgctcctgtcccccaggaacgctgggggtgctctgcgagattaatgaggatgactgcggcccaggcccaccgctggactcagggccccggtgcctacacaatggcacctgcgtggacctggtgggtggtttccgctgcacctgtcccccaggatacactggtttgcgctgcgaggcagacatcaatgagtgtcgctcaggtgcctgccacgcggcacacacccgggactgcctgcaggacccaggcggaggtttccgttgcctttgtcatgctggcttctcaggtcctcgctgtcagactgtcctgtctccctgcgagtcccagccatgccagcatggaggccagtgccgtcctagcccgggtcctgggggtgggctgaccttcacctgtcactgtgcccagccgttctggggtccgcgttgcgagcgggtggcgcgctcctgccgggagctgcagtgcccggtgggcgtcccatgccagcagacgccccgcgggccgcgctgcgcctgccccccagggttgtcgggaccctcctgccgcagcttcccggggtcgccgccgggggccagcaacgccagctgcgcggccgccccctgtctccacgggggctcctgccgccccgcgccgctcgcgcccttcttccgctgcgcttgcgcgcagggctggaccgggccgcgctgcgaggcgcccgccgcggcacccgaggtctcggaggagccgcggtgcccgcgcgccgcctgccaggccaagcgcggggaccagcgctgcgaccgcgagtgcaacagcccaggctgcggctgggacggcggcgactgctcgctgagcgtgggcgacccctggcggcaatgcgaggcgctgcagtgctggcgcctcttcaacaacagccgctgcgaccccgcctgcagctcgcccgcctgcctctacgacaacttcgactgccacgccggtggccgcgagcgcacttgcaacccggtgtacgagaagtactgcgccgaccactttgccgacggccgctgcgaccagggctgcaacacggaggagtgcggctgggatgggctggattgtgccagcgaggtgccggccctgctggcccgcggcgtgctggtgctcacagtgctgctgccgccggaggagctactgcgttccagcgccgactttctgcagcggctcagcgccatcctgcgcacctcgctgcgcttccgcctggacgcgcacggccaggccatggtcttcccttaccaccggcctagtcctggctccgaaccccgggcccgtcgggagctggcccccgaggtgatcggctcggtagtaatgctggagattgacaaccggctctgcctgcagtcgcctgagaatgatcactgcttccccgatgcccagagcgccgctgactacctgggagcgttgtcagcggtggagcgcctggacttcccgtacccactgcgggacgtgcggggggagccgctggagcctccagaacccagcgtcccgctgctgccactgctagtggcgggcgctgtcttgctgctggtcattctcgtcctgggtgtcatggtggcccggcgcaagcgcgagcacagcaccctctggttccctgagggcttctcactgcacaaggacgtggcctctggtcacaagggccggcgggaacccgtgggccaggacgcgctgggcatgaagaacatggccaagggtgagagcctgatgggggaggtggccacagactggatggacacagagtgcccagaggccaagcggctaaaggtagaggagccaggcatgggggctgaggaggctgtggattgccgtcagtggactcaacaccatctggttgctgctgacatccgcgtggcaccagccatggcactgacaccaccacagggcgacgcagatgctgatggcatggatgtcaatgtgcgtggcccagatggcttcaccccgctaatgctggcttccttctgtgggggggctctggagccaatgccaactgaagaggatgaggcagatgacacatcagctagcatcatctccgacctgatctgccagggggctcagcttggggcacggactgaccgtactggcgagactgctttgcacctggctgcccgttatgcccgtgctgatgcagccaagcggctgctggatgctggggcagacaccaatgcccaggaccactcaggccgcactcccctgcacacagctgtcacagccgatgcccagggtgtcttccagattctcatccgaaaccgctctacagacttggatgcccgcatggcagatggctcaacggcactgatcctggcggcccgcctggcagtagagggcatggtggaagagctcatcgccagccatgctgatgtcaatgctgtggatgagcttgggaaatcagccttacactgggctgcggctgtgaacaacgtggaagccactttggccctgctcaaaaatggagccaataaggacatgcaggatagcaaggaggagacccccctattcctggccgcccgcgagggcagctatgaggctgccaagctgctgttggaccactttgccaaccgtgagatcaccgaccacctggacaggctgccgcgggacgtagcccaggagagactgcaccaggacatcgtgcgcttgctggatcaacccagtgggccccgcagcccccccggtccccacggcctggggcctctgctctgtcctccaggggccttcctccctggcctcaaagcggcacagtcggggtccaagaagagcaggaggccccccgggaaggcggggctggggccgcaggggccccgggggcggggcaagaagctgacgctggcctgcccgggccccctggctgacagctcggtcacgctgtcgcccgtggactcgctggactccccgcggcctttcggtgggccccctgcttcccctggtggcttcccccttgaggggccctatgcagctgccactgccactgcagtgtctctggcacagcttggtggcccaggccgggcaggtctagggcgccagccccctggaggatgtgtactcagcctgggcctgctgaaccctgtggctgtgcccctcgattgggcccggctgcccccacctgcccctccaggcccctcgttcctgctgccactggcgccgggaccccagctgctcaacccagggacccccgtctccccgcaggagcggcccccgccttacctggcagtcccaggacatggcgaggagtacccggtggctggggcacacagcagccccccaaaggcccgcttcctgcgggttcccagtgagcacccttacctgaccccatcccccgaatcccctgagcactgggccagcccctcacctccctccctctcagactggtccgaatccacgcctagcccagccactgccactggggccatggccaccaccactggggcactgcctgcccagccacttcccttgtctgttcccagctcccttgctcaggcccagacccagctggggccccagccggaagttacccccaagaggcaagtgttggcctgagacgctcgtcagttcttagatcttgggggcctaaagagacccccgtcctgcctcctttctttctctgtctcttccttccttttagtctttttcatcctcttctctttccaccaaccctcctgcatccttgccttgcagcgtgaccgagataggtcatcagcccagggcttcagtcttcctttatttataatgggtgggggctaccacccaccctctcagtcttgtgaagagtctgggacctccttcttccccacttctctcttccctcattcctttctctctccttctggcctctcatttccttacactctgacatgaatgaattattattatttttctttttctttttttttttacattttgtatagaaacaaattcatttaaacaaacttattattattattttttacaaaatatatatatggagatgctccctccccctgtgaaccccccagtgcccccgtggggctgagtctgtgggcccattcggccaagctggattctgtgtacctagtacacaggcatgactgggatcccgtgtaccgagtacacgacccaggtatgtaccaagtaggcacccttgggcgcacccactggggccaggggtcgggggagtgttgggagcctcctccccaccccacctccctcacttcactgcattccagattggacatgttccatagccttgctggggaagggcccactgccaactccctctgccccagccccacccttggccatctccctttgggaactagggggctgctggtgggaaatgggagccagggcagatgtatgcattcctttatgtccctgtaaatgtgggactacaagaagaggagctgcctgagtggtactttctcttcctggtaatcctctggcccagccttatggcagaatagaggtatttttaggctatttttgtaatatggcttctggtcaaaatccctgtgtagctgaattcccaagccctgcattgtacagccccccactcccctcaccacctaataaaggaatagttaacactcaaaaaaaaaaaaaaaaaaa

SEQ ID NO: 3 - protein, 1532 aa, Homo sapiens
APPCLDGSPCANGGRCTQLPSREAACLCPPGWVGERCQLEDPCHSGPCAGRGVCQSSVVAGTARFSCRCPRGFRGPDCSLPDPCLSSPCAHGARCSVGPDGRFLCSCPPGYQGRSCRSDVDECRVGEPCRHGGTCLNTPGSFRCQCPAGYTGPLCENPAVPCAPSPCRNGGTCRQSGDLTYDCACLPGFEGQNCEVNVDDCPGHRCLNGGTCVDGVNTYNCQCPPEWTGQFCTEDVDECQLQPNACHNGGTCFNTLGGHSCVCVNGWTGESCSQNIDDCATAVCFHGATCHDRVASFYCACPMGKTGLLCHLDDACVSNPCHEDAICDTNPVNGRAICTCPPGFTGGACDQDVDECSIGANPCEHLGRCVNTQGSFLCQCGRGYTGPRCETDVNECLSGPCRNQATCLDRIGQFTCICMAGFTGTYCEVDIDECQSSPCVNGGVCKDRVNGFSCTCPSGFSGSTCQLDVDECASTPCRNGAKCVDQPDGYECRCAEGFEGTLCDRNVDDCSPDPCHHGRCVDGIASFSCACAPGYTGTRCESQVDECRSQPCRHGGKCLDLVDKYLCRCPSGTTGVNCEVNIDDCASNPCTFGVCRDGINRYDCVCQPGFTGPLCNVEINECASSPCGEGGSCVDGENGFRCLCPPGSLPPLCLPPSHPCAHEPCSHGICYDAPGGFRCVCEPGWSGPRCSQSLARDACESQPCRAGGTCSSDGMGFHCTCPPGVQGRQCELLSPCTPNPCEHGGRCESAPGQLPVCSCPQGWQGPRCQQDVDECAGPAPCGPHGICTNLAGSFSCTCHGGYTGPSCDQDINDCDPNPCLNGGSCQDGVGSFSCSCLPGFAGPRCARDVDECLSNPCGPGTCTDHVASFTCTCPPGYGGFHCEQDLPDCSPSSCFNGGTCVDGVNSFSCLCRPGYTGAHCQHEADPCLSRPCLHGGVCSAAHPGFRCTCLESFTGPQCQTLVDWCSRQPCQNGGRCVQTGAYCLCPPGWSGRLCDIRSLPCREAAAQIGVRLEQLCQAGGQCVDEDSSHYCVCPEGRTGSHCEQEVDPCLAQPCQHGGTCRGYMGGYMCECLPGYNGDNCEDDVDECASQPCQHGGSCIDLVARYLCSCPPGTLGVLCEINEDDCGPGPPLDSGPRCLHNGTCVDLVGGFRCTCPPGYTGLRCEADINECRSGACHAAHTRDCLQDPGGGFRCLCHAGFSGPRCQTVLSPCESQPCQHGGQCRPSPGPGGGLTFTCHCAQPFWGPRCERVARSCRELQCPVGVPCQQTPRGPRCACPPGLSGPSCRSFPGSPPGASNASCAAAPCLHGGSCRPAPLAPFFRCACAQGWTGPRCEAPAAAPEVSEEPRCPRAACQAKRGDQRCDRECNSPGCGWDGGDCSLSVGDPWRQCEALQCWRLFNNSRCDPACSSPACLYDNFDCHAGGRERTCNPVYEKYCADHFADGRCDQGCNTEECGWDGLDCASEVPALLARGVLVLTVLLPPEELLRSSADFLQRLSAILRTSLRFRLDAHGQAMVFPYHRPSPGSEPRARR

SEQ ID NO: 4 - protein, 1533 aa, Mus musculus
APPCLDGSPCANGGRCTHQQPSLEAACLCLPGWVGERCQLEDPCHSGPCAGRGVCQSSVVAGTARFSCRCLRGFQGPDCSQPDPCVSRPCVHGAPCSVGPDGRFACACPPGYQGQSCQSDIDECRSGTTCRHGGTCLNTPGSFRCQCPLGYTGLLCENPVVPCAPSPCRNGGTCRQSSDVTYDCACLPGFEGQNCEVNVDDCPGHRCLNGGTCVDGVNTYNCQCPPEWTGQFCTEDVDECQLQPNACHNGGTCFNLLGGHSCVCVNGWTGESCSQNIDDCATAVCFHGATCHDRVASFYCACPMGKTGLLCHLDDACVSNPCHEDAICDTNPVSGRAICTCPPGFTGGACDQDVDECSIGANPCEHLGRCVNTQGSFLCQCGRGYTGPRCETDVNECLSGPCRNQATCLDRIGQFTCICMAGFTGTYCEVDIDECQSSPCVNGGVCKDRVNGFSCTCPSGFSGSMCQLDVDECASTPCRNGAKCVDQPDGYECRCAEGFEGTLCERNVDDCSPDPCHHGRCVDGIASFSCACAPGYTGIRCESQVDECRSQPCRYGGKCLDLVDKYLCRCPPGTTGVNCEVNIDDCASNPCTFGVCRDGINRYDCVCQPGFTGPLCNVEINECASSPCGEGGSCVDGENGFHCLCPPGSLPPLCLPANHPCAHKPCSHGVCHDAPGGFRCVCEPGWSGPRCSQSLAPDACESQPCQAGGTCTSDGIGFRCTCAPGFQGHQCEVLSPCTPSLCEHGGHCESDPDRLTVCSCPPGWQGPRCQQDVDECAGASPCGPHGTCTNLPGNFRCICHRGYTGPFCDQDIDDCDPNPCLHGGSCQDGVGSFSCSCLDGFAGPRCARDVDECLSSPCGPGTCTDHVASFTCACPPGYGGFHCEIDLPDCSPSSCFNGGTCVDGVSSFSCLCRPGYTGTHCQYEADPCFSRPCLHGGICNPTHPGFECTCREGFTGSQCQNPVDWCSQAPCQNGGRCVQTGAYCICPPGWSGRLCDIQSLPCTEAAAQMGVRLEQLCQEGGKCIDKGRSHYCVCPEGRTGSHCEHEVDPCTAQPCQHGGTCRGYMGGYVCECPAGYAGDSCEDNIDECASQPCQNGGSCIDLVARYLCSCPPGTLGVLCEINEDDCDLGPSLDSGVQCLHNGTCVDLVGGFRCNCPPGYTGLHCEADINECRPGACHAAHTRDCLQDPGGHFRCVCHPGFTGPRCQIALSPCESQPCQHGGQCRHSLGRGGGLTFTCHCVPPFWGLRCERVARSCRELQCPVGIPCQQTARGPRCACPPGLSGPSCRVSRASPSGATNASCASAPCLHGGSCLPVQSVPFFRCVCAPGWGGPRCETPSAAPEVPEEPRCPRAACQAKRGDQNCDRECNTPGCGWDGGDCSLNVDDPWRQCEALQCWRLFNNSRCDPACSSPACLYDNFDCYSGGRDRTCNPVYEKYCADHFADGRCDQGCNTEECGWDGLDCASEVPALLARGVLVLTVLLPPEELLRSSADFLQRLSAILRTSLRFRLDARGQAMVFPYHRPSPGSESRVRR

SEQ ID NO: 5 - protein, 183 aa, Homo sapiens
HSHRDFQPVLHLVALNSPLSGGMRGIRGADFQCFQQARAVGLAGTFRAFLSSRLQDLYSIVRRADRAAVPIVNLKDELLFPSWEALFSGSEGPLKPGARIFSFDGKDVLRHPTWPQKSVWHGSDPNGRRLTESYCETWRTEAPSATGQASSLLGGRLLGQSAASCHHAYIVLCIENSFMTASK

SEQ ID NO: 6 - DNA, 549 bp, Homo sapiens
  source 1..549: mol_type = genomic DNA
cacagccaccgcgacttccagccggtgctccacctggttgcgctcaacagccccctgtcaggcggcatgcggggcatccgcggggccgacttccagtgcttccagcaggcgcgggccgtggggctggcgggcaccttccgcgccttcctgtcctcgcgcctgcaggacctgtacagcatcgtgcgccgtgccgaccgcgcagccgtgcccatcgtcaacctcaaggacgagctgctgtttcccagctgggaggctctgttctcaggctctgagggtccgctgaagcccggggcacgcatcttctcctttgacggcaaggacgtcctgaggcaccccacctggccccagaagagcgtgtggcatggctcggaccccaacgggcgcaggctgaccgagagctactgtgagacgtggcggacggaggctccctcggccacgggccaggcctcctcgctgctggggggcaggctcctggggcagagtgccgcgagctgccatcacgcctacatcgtgctctgcattgagaacagcttcatgactgcctccaag

SEQ ID NO: 7 - protein, 259 aa, Homo sapiens
MSEVPVARVWLVLLLLTVQVGVTAGAPWQCAPCSAEKLALCPPVSASCSEVTRSAGCGCCPMCALPLGAACGVATARCARGLSCRALPGEQQPLHALTRGQGACVQESDASAPHAAEAGSPESPESTEITEEELLDNFHLMAPSEEDHSILWDAISTYDGSKALHVTNIKKWKEPCRIELYRVVESLAKAQETSGEEISKFYLPNCNKNGFYHSRQCETSMDGEAGLCWCVYPWNGKRIPGSPEIRGDPNCQIYFNVQN

SEQ ID NO: 8 - DNA, 1660 bp, Homo sapiens
  source 1..1660: mol_type = genomic DNA
ggtgcactagcaaaacaaacttattttgaacactcagctcctagcgtgcggcgctgccaatcattaacctcctggtgcaagtggcgcggcctgtgccctttataaggtgcgcgctgtgtccagcgagcatcggccaccgccatcccatccagcgagcatctgccgccgcgccgccgccaccctcccagagagcactggccaccgctccaccatcacttgcccagagtttgggccaccgcccgccgccaccagcccagagagcatcggcccctgtctgctgctcgcgcctggagatgtcagaggtccccgttgctcgcgtctggctggtactgctcctgctgactgtccaggtcggcgtgacagccggcgctccgtggcagtgcgcgccctgctccgccgagaagctcgcgctctgcccgccggtgtccgcctcgtgctcggaggtcacccggtccgccggctgcggctgttgcccgatgtgcgccctgcctctgggcgccgcgtgcggcgtggcgactgcacgctgcgcccggggactcagttgccgcgcgctgccgggggagcagcaacctctgcacgccctcacccgcggccaaggcgcctgcgtgcaggagtctgacgcctccgctccccatgctgcagaggcagggagccctgaaagcccagagagcacggagataactgaggaggagctcctggataatttccatctgatggccccttctgaagaggatcattccatcctttgggacgccatcagtacctatgatggctcgaaggctctccatgtcaccaacatcaaaaaatggaaggagccctgccgaatagaactctacagagtcgtagagagtttagccaaggcacaggagacatcaggagaagaaatttccaaattttacctgccaaactgcaacaagaatggattttatcacagcagacagtgtgagacatccatggatggagaggcgggactctgctggtgcgtctacccttggaatgggaagaggatccctgggtctccagagatcaggggagaccccaactgccagatatattttaatgtacaaaactgaaaccagatgaaataatgttctgtcacgtgaaatatttaagtatatagtatatttatactctagaacatgcacatttatatatatatgtatatgtatatatatatagtaactactttttatactccatacataacttgatatagaaagctgtttatttattcactgtaagtttattttttctacacagtaaaaacttgtactatgttaataacttgtcctatgtcaatttgtatatcatgaaacacttctcatcatattgtatgtaagtaattgcatttctgctcttccaaagctcctgcgtctgtttttaaagagcatggaaaaatactgcctagaaaatgcaaaatgaaataagagagagtagtttttcagctagtttgaaggaggacggttaacttgtatattccaccattcacatttgatgtacatgtgtagggaaagttaaaagtgttgattacataatcaaagctacctgtggtgatgttgccacctgttaaaatgtacactggatatgttgttaaacacgtgtctataatggaaacatttacaataaatattctgcatggaaatactgttaaaaaaaaaaa

SEQ ID NO: 9 - protein, 480 aa, Homo sapiens
MQIPRAALLPLLLLLLAAPASAQLSRAGRSAPLAAGCPDRCEPARCPPQPEHCEGGRARDACGCCEVCGAPEGAACGLQEGPCGEGLQCVVPFGVPASATVRRRAQAGLCVCASSEPVCGSDANTYANLCQLRAASRRSERLHRPPVIVLQRGACGQGQEDPNSLRHKYNFIADVVEKIAPAVVHIELFRKLPFSKREVPVASGSGFIVSEDGLIVTNAHVVTNKHRVKVELKNGATYEAKIKDVDEKADIALIKIDHQGKLPVLLLGRSSELRPGEFVVAIGSPFSLQNTVTTGIVSTTQRGGKELGLRNSDMDYIQTDAIINYGNSGGPLVNLDGEVIGINTLKVTAGISFAIPSDKIKKFLTESHDRQAKGKAITKKKYIGIRMMSLTSSKAKELKDRHRDFPDVISGAYIIEVIPDTPAEAGGLKENDVIISINGQSVVSANDVSDVIKRESTLNMVVRRGNEDIMITVIPEEIDP

SEQ ID NO: 10 - protein, 22 aa, synthetic construct
  REGION 1..22: note = positions 1-22 of (SEQ ID NO: 9) which correspond to the signal peptide
MQIPRAALLPLLLLLLAAPASA

SEQ ID NO: 11 - protein, 161 aa, synthetic construct
GSGFIVSEDGLIVTNAHVVTNKHRVKVELKNGATYEAKIKDVDEKADIALIKIDHQGKLPVLLLGRSSELRPGEFVVAIGSPFSLQNTVTTGIVSTTQRGGKELGLRNSDMDYIQTDAIINYGNSGGPLVNLDGEVIGINTLKVTAGISFAIPSDKIKKFL

SEQ ID NO: 12 - DNA, 2138 bp, Homo sapiens
  source 1..2138: mol_type = genomic DNA
caatgggctgggccgcgcggccgcgcgcactcgcacccgctgcccccgaggccctcctgcactctccccggcgccgctctccggccctcgccctgtccgccgccaccgccgccgccgccagagtcgccatgcagatcccgcgcgccgctcttctcccgctgctgctgctgctgctggcggcgcccgcctcggcgcagctgtcccgggccggccgctcggcgcctttggccgccgggtgcccagaccgctgcgagccggcgcgctgcccgccgcagccggagcactgcgagggcggccgggcccgggacgcgtgcggctgctgcgaggtgtgcggcgcgcccgagggcgccgcgtgcggcctgcaggagggcccgtgcggcgaggggctgcagtgcgtggtgcccttcggggtgccagcctcggccacggtgcggcggcgcgcgcaggccggcctctgtgtgtgcgccagcagcgagccggtgtgcggcagcgacgccaacacctacgccaacctgtgccagctgcgcgccgccagccgccgctccgagaggctgcaccggccgccggtcatcgtcctgcagcgcggagcctgcggccaagggcaggaagatcccaacagtttgcgccataaatataactttatcgcggacgtggtggagaagatcgcccctgccgtggttcatatcgaattgtttcgcaagcttccgttttctaaacgagaggtgccggtggctagtgggtctgggtttattgtgtcggaagatggactgatcgtgacaaatgcccacgtggtgaccaacaagcaccgggtcaaagttgagctgaagaacggtgccacttacgaagccaaaatcaaggatgtggatgagaaagcagacatcgcactcatcaaaattgaccaccagggcaagctgcctgtcctgctgcttggccgctcctcagagctgcggccgggagagttcgtggtcgccatcggaagcccgttttcccttcaaaacacagtcaccaccgggatcgtgagcaccacccagcgaggcggcaaagagctggggctccgcaactcagacatggactacatccagaccgacgccatcatcaactatggaaactcgggaggcccgttagtaaacctggacggtgaagtgattggaattaacactttgaaagtgacagctggaatctcctttgcaatcccatctgataagattaaaaagttcctcacggagtcccatgaccgacaggccaaaggaaaagccatcaccaagaagaagtatattggtatccgaatgatgtcactcacgtccagcaaagccaaagagctgaaggaccggcaccgggacttcccagacgtgatctcaggagcgtatataattgaagtaattcctgataccccagcagaagctggtggtctcaaggaaaacgacgtcataatcagcatcaatggacagtccgtggtctccgccaatgatgtcagcgacgtcattaaaagggaaagcaccctgaacatggtggtccgcaggggtaatgaagatatcatgatcacagtgattcccgaagaaattgacccataggcagaggcatgagctggacttcatgtttccctcaaagactctcccgtggatgacggatgaggactctgggctgctggaataggacactcaagacttttgactgccattttgtttgttcagtggagactccctggccaacagaatccttcttgatagtttgcaggcaaaacaaatgtaatgttgcagatccgcaggcagaagctctgcccttctgtatcctatgtatgcagtgtgctttttcttgccagcttgggccattcttgcttagacagtcagcatttgtctcctcctttaactgagtcatcatcttagtccaactaatgcagtcgatacaatgcgtagatagaagaagccccacgggagccaggatgggactggtcgtgtttgtgcttttctccaagtcagcacccaaaggtcaatgcacagagaccccgggtgggtgagcgctggcttctcaaacggccgaagttgcctcttttaggaatctctttggaattgggagcacgatgactctgagtttgagctattaaagtacttcttacacattgcaaaaaaaaaaaaaaaaaa

SEQ ID NO: 13 - DNA, 1043 bp, synthetic construct
  source 1..1043: mol_type = other DNA
aaatgtagtcctaggctcctggcctggctgagtgagttttagcggttttttgtttgtttgttttgtttgagacagggtcttactctgttgcccaggctggagcgcagtggtacgatttcggctaagtgcaacctcagcctcctgggttgaagcgattttcctgcttcagcctctcgaatagctgggactacaggcgtgcgccaccacgctcagctaatttttgtattttcagtagagtccgggtttcaccatgttggccaggctggtctccaactcttgacctcaagtgatccatccacctctgcctctcaaagtgctgggattacaggccggagccaccgcgcccggtccgacttttaggtttgtgaatgttttagatcagagtcctggggaagcttggtgcatgggcttgcatttgtgcgtccgtggctgtgggtccatgagcctctcaggacgtgactggcctcagtttccagagtttctgggaggctgtgttttttgtcccggctccagaggtgtccggctctgggtgtgtactgggggatggggatggggtgcgtgggcgttcacgaggttgggtgtgcccgccactccgggttctgcccgcgtctcactgcatgctcggcctgggtttccgagggtccgcgcgtcccaggctgtgcgggtggagggtgggcagggaccccgggaggccgggcggggggcgggggcgcgctgggccggccccggggcggggcgagccttcgagggctgggggcggggcggcccggccgcctcacttcggcgaagttggcggcgcggaggctggcccgggacgcgcccggagcccagggaaggagggaggaggggagggtcgcggccggccgccatggggccgggggcccgtggccgccgccgccgccgtcgcccgatgtcgccgccaccgccaccgccacccgtgcgggcgctgcccctgctgctgctgctagcggggccgggggctgcaggtgaggggccgggacctggcggatgggacgagggcggcagagggggagtgcaagaac

SEQ ID NO: 14 - DNA, 1068 bp, synthetic construct
  source 1..1068: mol_type = other DNA
gctttgagacgtgaaaaggaatgtatctgtcaatatccagagatatccaatctgtgctggatgaccttgaccaaatcacattccctctctgtgccaattcacccctgggccttggtttgtccttctgaaaaacggctgatcatcttggaggatctttccatctcctccatgtcacctcttctcagcagtgctctctgattctctcaggcttccgttaggggtccttctaaatgcactctcagcctcctgtctgccgaccgggcacctgcttgcccaatgaatatcttgctcactagcctggtgcttctgaggtcaggaccacgtcttgctactgtcccctgcacccagtacagtgttagaatgtagtaaatgctcagtatgcttgttcaatgaatgaaggttgactgaatcaaccacagagatgatcagagtctcctttacagatggggaaactgaggcactgagaaatgatctccctgggtgccatggcaaggggaggccgccacccgacatgggcatcgggaaagtggggccgaggccactctttgacaaaagtgactcagcagctggcggctggcgtgggctgtggaatttctaagcatcccctcacatcctgagcgaacgggcgatgggagggagtaagcagagtgaaaagaagaaaaacaagaaacttgggggaaaagaaagagagagggggaaaaaaactgaaattgaaaacagacgcacgcgtccaccctccctgccccgccgcccccccttctcccagctgggccaatcagaagccaccctgcagcctttcccccaaagtggcgggggaagtgggggggcatggaagcccatagtggtgtgagctcctgaggctgccagcagccagcagtgactgcccgccctatctgggacccaggatcgctctgtgagcaacttggagccagagaggagatcaacaaggaggaggagagagccggcccctcagccctgctgcccagcagcagcctgtgctcgccctgcccaacgcagacagccagacccagggcggcccctctggcggctctgctcctcccgaaggat

SEQ ID NO: 15 - DNA, 940 bp, synthetic construct
  source 1..940: mol_type = other DNA
gccaggccttatagaccctcttattgacactgcccactggatggggaccggagttggactcagcttctgccgaaccctcaaatcccagccccaactaaagcatataactcaagacctacctgcactgaaagctcttctcaacctgagcagggtggtccaattgaaagggtgggtctgaccacctctcctgcacccatgcgggttggcagaggtgtgcaggatctgccacttaccattcaccatgtggccttgaggaagacgcactcggggcctcagtttcctcatctataaaatggggatgtaattacaccctcacactgtagctgtgagtattcaatgagagcactgcaaagggcctggtgtggagtaggtcctcaggaaaggttggatcccatgtcccatcagagctaaaagccccaggaggagagggtggctggtttgtccccacaaacccctgggattcccggctccccagccccttgcccctctctccagccagactctattgaactccccctcttctcaaactcggggccagagaacagtgaagtaggagcagccgtaagtccgggcagggtcctgtccataaaaggcttttcccgggccggctccccgccggcagcgtgccccgccccggcccgctccatctccaaagcatgcagagaatgtctcggcagccccggtagactgctccaacttggtgtctttccccaaatatggagcctgtgtggagtcactgggggagccgggggtggggagcggagccggcttcctctagcagggagggggccgaggagcgagccagtgggggaggctgacatcaccacggcggcagccctttaaacccctcacccagccagcgccccatcctgtctgtccgaacccagacacaagtcttcactccttcctgcgagccctgaggaagccttgtgagtgcatt

SEQ ID NO: 16 - DNA, 1089 bp, synthetic construct
  source 1..1089: mol_type = other DNA
agtaacattgccctttctcctcctgtttgggactaggctgatgttgctgcctggaagggagccagcagaagggccccagcctgaagctgttaggtagaagccaaatccagggccagatttccaggaggcagcctcgggaagttgaaacacccggattcaggggtcaggaggcctgggcttctggcaccaaacggccagggacctactttccacctggagtcttgtaagagccactttcagcttgagctgcactttcgtcctccatgaaatgggggaggggatgctcctcacccaccttgcaaggttattttgaggcaaatgtcatggcgggactgagaattcttctgccctgcgaggaaatccagacatctctcccttacagacagggagactgaggtgaggcccttccaggcagagaaggtcactgttgcagccatgggcagtgccccacaggacctcgggtggtgcctctggagtctggagaagttcctaggggacctccgaggcaaagcagcccaaaagccgcctgtgagggtggctggtgtctgtccttcctcctaaggctggagtgtgcctgtggaggggtctcctgaactcccgcaaaggcagaaaggagggaagtaggggctgggacagttcatgcctcctccctgagggggtctcccgggctcggctcttggggccagagttcagggtgtctgggcctctctatgactttgttctaagtctttagggtggggctggggtctggcccagctgcaagggccccctcacccctgccccagagaggaacagccccgcacgggccctttaagaaggttgagggtgggggcaggtgggggagtccaagcctgaaacccgagcgggcgcgcgggtctgcgcctgccccgcccccggagttaagtgcgcggacacccggagccggcccgcgcccaggagcagagccgcgctcgctccactcagctcccagctcccaggactccgctggctcctcgcaagtcctgccgcccagcccgccgggatgcagtccgggccgcggcccccacttccagcccccggcctggccttggctttgaccctgactatgtt

SEQ ID NO: 17 - DNA, 1056 bp, synthetic construct
  source 1..1056: mol_type = other DNA
ccttgagtgataggaagcaggaaaagtttgaaggagaaggaaagacaagcctaggcaaggatggctatggtggggaggtacaaagaattacaaagcattagtcatccaagcaaagtataatcagaagaccactgacagaattaacagagttcagagataatatcattatactcaatccctactaagattttggtgtttggtctcctagatcacttttttagtaccttcacagacatatggattcataaagaatatatagtaattattcatatttgttttgatttatataaacagtaatcttgtctctcagctccagaaaaccttgcctggtgcttcctgaaggctcacaacatcccagcccggcctccatcacttacttataatgtgttttggggtcttgagggcagggagctttctctttcttctctgaattcctagcatccagctgagtgcctggcacttggtaagtattcacatacacattccttgaattccctagatttcagtttccttcactgtgaaacaaagtgggctaaacgatctccaggagcccttacagtcctaagaatctatgactttgaccgatctgaagtggtccaagacactttgcttcacaggaagctcacatcttccttgtgattggcctgagattaggagttcaaacaaccaatgacctgtgcacagtttagggcctaaccctgccctggccggttagcaagagagtgttgtgataagagcagatgaaagccttatgccacagtagtgcctgtagcagagaaaggattccctcctccacctcagggcccactgctgctgctctccagcctgcagtttcctctaaggctctggattggctggaagagcaacagagggctgggaaagagcttctatatatacctcaggaggaaaggcatcccagacagttttgaagttttcaaagactggctctgctgttaagaagttgtacttaaagcggaggagctaagccacctgccaaaatgtgcaaaggacttgcagctttgccccactcatgcctggaaaggtgagaaccaagtt

SEQ ID NO: 18 - DNA, 937 bp, synthetic construct
  source 1..937: mol_type = other DNA
agggaagttgttgaagggaactcgcagactctgggggcacacccactttctccttggatcttgacacttgcatcttgtaaataacgtaattatcaccgccaccgccttcccccattttgtagctatggacaccaagtctcagagaagtgaagtgacttgcccaaggtcacgcagctggcgagtggcgcacaggggagggggacagctgaaataatcacagtgggcttatttttaatttttatttgtattttggtcgtggtgatgtgggtggaggtggagatggcaagttgggaaaagtaaaaacttccccttcctgcacggttcccagcaagggtgggggcctcctgtcttgcactttgcaaagttcaagaaatcccctttccctacccttcacgctgcacagccggccctctttccagacagtgcgatgccaataaaatgggaagtggggtgggagatgtcaagtcagatccaccacagccccgacacggggaggaagaggttaaagcctttgcggccggaaccgactcagggaagacgttctcaagcatcccgcacagacactgcctgctcgaccccctttctctagggatccggagcgtctgcgaccgcctggggccggggctgagactcccgtccctgtgcgcacctgttccgtgcgcccttgtgcggtgcgcacctgttccgtgcacccttgtcccgagcgccccagctccttgcgctcccgccgggggtgcgccctgcagggggcgcggcgagggggccgcgagggaccctccccaactccaccccttcggcctcctcccctttcccagccgcgggcagctccgggtctataaagagaggcgtccgaggacgcgcagggagatttggacgctccggcctgggaggtgcgtcagatccgagctcgccatccagtttcctctccactagtccc

(19) United States
(12) Patent Application Publication
(10) Pub. No.: US 2025/0064977 A1
(43) Pub. Date: Feb. 27, 2025

(54) Nucleic Acid-Based Compositions and Methods for Treating Small Vessel Diseases

(71) Applicant: The Schepens Eye Research Institute, Inc., Boston, MA (US)

(72) Inventor: Joseph F. Arboleda-Velasquez, Newton, MA (US)

(21) Appl. No.: 18/672,136

(22) Filed: May 23, 2024

Related U.S. Application Data
(63) Continuation of application No. 16/499,234, filed on Sep. 27, 2019, now abandoned.
(60) Provisional application No. 62/477,271, filed on Mar. 27, 2017.

(51) Int. Cl. A61K 48/00; C12N 15/85; C12N 15/90

(52) U.S. Cl. CPC A61K 48/0058; A61K 48/0075; C12N 15/85; C12N 15/907; C12N 2800/107; C12N 2830/008

(57) ABSTRACT

The present subject matter provides, inter alia compositions, formulations, and methods for inhibiting, treating, and preventing small vessel diseases.

### RELATED APPLICATIONS

This application is a continuation of U.S. patent application Ser. No. 16/499,234, filed Sep. 27, 2019, which is filed under national stage application filed under 35 U.S.C. § 371, of International Patent Application No. PCT/US2018/024407, filed Mar. 26, 2018, which claims the benefit of and priority to U.S. Provisional Patent Application No. 62/477,271, filed Mar. 27, 2017, the entire contents of each of which are incorporated herein by reference.

### STATEMENT AS TO FEDERALLY SPONSORED RESEARCH

This invention was made with government support under EY021624 and NS100121 awarded by the National Institutes of Health. The government has certain rights in the invention.

### SEQUENCE LISTING

This application contains a Sequence Listing that has been submitted electronically as an XML file named “006330309002_SL_ST26.XML.” The XML file, created on May 22, 2024, is 41,953 bytes in size. The material in the XML file is hereby incorporated by reference in its entirety.

### FIELD OF THE INVENTION

The present invention relates to small vessel diseases.

### BACKGROUND

Cerebral small vessel disease (SVD) is characterized by progressive degeneration of the small penetrating arteries and arterioles of the brain (Rosenberg et al., 2015, J Cereb Blood Flow Metab). Pathological changes in the small vessels include mural cell loss, thickening of basement membranes, and accumulation of deposits in vessel walls (Iadecola, 2013, Neuron 80:844-866; Rosenberg et al., 2015, J Cereb Blood Flow Metab). SVD is responsible for the vast majority of silent brain infarcts, is the most common cause of vascular cognitive impairment and vascular dementia, and is a major risk factor for clinically overt stroke (Hakim, 2014, Nature 510:S12; Iadecola, 2013, Neuron 80:844-866; Thompson and Hakim, 2009, Stroke 40:e322-330). There is increasing evidence that SVD exacerbates Alzheimer's disease pathology and vice versa; indeed, it is now clear that the most common etiology of dementia in older people includes a mixture of vascular (particularly small vessel) disease and Alzheimer's pathology (Snyder et al., 2015, Alzheimers Dement 11:710-717). SVD is accelerated and exacerbated by cardiovascular risk factors, including high blood pressure and diabetes, but one of the strongest risk factors for SVD is age (Iadecola, 2013, Neuron 80:844-866). Currently, SVD is treated via modification of cardiovascular risk factors without addressing vascular degeneration directly.

New compositions and methods for the treatment of SVDs are needed.

### SUMMARY OF THE INVENTION

Provided herein are, inter alia, compositions, vectors, formulations, and methods for inhibiting, treating, and preventing SVDs. Aspects of the present subject matter relate to increasing the level of functional NOTCH3 in a subject for the treatment and prevention of a wide variety of SVDs including but not limited to cerebral small vessel disease, cerebral autosomal recessive arteriopathy with subcortical infarcts and leukoencephalopathy (CARASIL), age-related macular degeneration (AMD), cerebral autosomal-dominant arteriopathy with subcortical infarcts and leukoencephalopathy (CADASIL), a NOTCH3 loss-of-function-associated SVD (e.g., a SVD associated with a mutation that reduces the expression and/or activity of NOTCH3), nephropathy or another SVD of the kidney, microangiopathy, proximal 19p13.12 microdeletion syndrome, myocardial ischemia, heart failure, Alagille syndrome, familial tetralogy of Fallot, patent ductus arteriosus, a cerebral cavernous malformation, a HTRA1-associated small vessel disease, and diabetic retinopathy.

The present subject matter includes a method for treating or preventing a SVD in a subject, comprising genetically modifying the subject to increase Neurogenic Locus Notch Homolog Protein 3 (NOTCH3) expression or activity in the subject. In some embodiments, genetically modifying the subject comprises replacing a mutant NOTCH3 gene with a purified wild-type or normal, unmutated NOTCH3 gene in the subject or adding an additional copy of a NOTCH3 gene, the additional copy comprising a normal unmutated nucleic acid sequence (e.g., a human wild-type sequence). In certain embodiments, a nucleotide sequence that encodes wild-type NOTCH3 protein is inserted into the genome of a subject.

In various embodiments, genetically modifying the subject comprises replacing the mutant NOTCH3 gene, or a mutated portion thereof, with a NOTCH3 gene or a corresponding portion of a NOTCH3 gene that does not comprise the mutation. In certain embodiments, genetically modifying the subject comprises expressing an exogenous NOTCH3 gene in the subject. In certain embodiments, the exogenous NOTCH3 gene is part of a genetic construct. In some embodiments, the genetic construct comprises a coding sequence that encodes NOTCH3 and a promoter, wherein the coding sequence is operably linked to the promoter. In some embodiments, genetically modifying the subject comprises administering a non-viral vector that comprises the genetic construct to the subject. In various embodiments, the non-viral vector comprises a plasmid. In certain embodiments, the plasmid is administered to the subject in a liposome. In some embodiments, genetically modifying the subject comprises administering a viral vector that comprises the genetic construct to the subject. In some embodiments, the viral vector comprises a retroviral vector, a adeno-associated viral vector, or a poxvirus vector. In various embodiments, altering the NOTCH3 gene comprises altering a promoter, enhancer, or other regulatory element of the gene, or an exon, an intron, or an intron-exon splice site of the gene.

In some embodiments, altering the NOTCH3 gene comprises the administration of (i) a Cas protein, a zinc finger nuclease (ZFN), or a transcription activator-like effector-based nuclease (TALEN), or (ii) an expression vector encoding a Cas protein, a ZFN, or a TALEN, to the subject. For example, the gene is altered using a CRISPR-Cas9 system. In various embodiments, a mutated NOTCH3 gene or a portion thereof is replaced. In some embodiments, a mutated NOTCH3 gene is mutated with a substitution such that the mutation is removed (i.e., reverted). In some embodiments, the expression of a NOTCH3 gene is increased. In certain embodiments, an exogenous polynucleotide that expresses NOTCH3 is administered to a subject.

In various embodiments, genetically modifying the subject comprises inserting a copy of a wild-type human NOTCH3 sequence such as the full-length wild-type sequence into the subject [e.g., directly, or into cells (e.g., vascular smooth muscle cells, mural cells, or pericytes) of the subject that are then administered to the subject]. In some examples, the therapeutic gene is delivered to pericytes cells or tissue of the brain, e.g., at the blood brain barrier.

In certain embodiments, a genetic construct is delivered into cells of the subject. For example, the construct may be delivered using a viral vector. In another example, the construct comprises naked DNA, e.g., in the form of a non-viral vector such as a plasmid. In various embodiments, the construct comprises a nucleic acid sequence that encodes NOTCH3 operably linked to a promoter. In some embodiments, the promoter is a tissue-specific or a cell-specific promoter. The promoter may be, e.g., constitutively active or may direct expression in a specific cell type such as mural cells (e.g., pericyte and vascular smooth muscle cells), pericytes (e.g., a desmin, a NOTCH3, an alpha-smooth muscle actin, a PDGFRβ, or a CSPG4 promoter), vascular smooth muscle cells (e.g., a SM22 promoter), or endothelial cells (e.g., a Tie2, Fli-1, vascular endothelial-cadherin, endoglin, Flt-1, or an intercellular adhesion molecule 2 promoter). In various embodiments, some of these promoters are combined to achieve cell type specificity. In various embodiments, the promoter comprises a desmin or an alpha-smooth muscle actin (α-SMA) promoter. In some embodiments, the promoter comprises a SM22 promoter. In certain embodiments, the promoter comprises a SM22a promoter. In various embodiments, the promoter comprises a Tie2, Fli-1, vascular endothelial-cadherin (VE-cadherin), endoglin, Flt-1, or intercellular adhesion molecule 2 promoter (ICAM-2) promoter. In certain embodiments, the promoter is specific for smooth muscle cells and pericytes [e.g., a NOTCH3 promoter or a platelet-derived growth factor receptor beta gene (PDGFRβ) promoter]. In some embodiments, the promoter is a NOTCH3 promoter (which is a mural cell-specific promoter rather than an endothelial cell-specific promoter). In embodiments, the promoter is a PDGFRβ promoter. In certain embodiments, the construct is administered to the subject in a liposome. In some embodiments, a non-viral vector (e.g., a plasmid that comprises a construct) is combined with an agent that facilitates its entry into cells such as a condensing agent. In some embodiments, the vector is condensed with an agent such as polyethyleneimine (PEI), poly-L-lysine (PLL) a polyamidoamine (PAMAM) dendrimer, spermidine, spermine, or cobalthexamine. In some embodiments, a Micelle-Like Nanoparticle (MNP) that comprises the vector is administered to the subject. In some embodiments, the promoter comprises a NOTCH3 promoter. In certain embodiments, the promoter comprises a MYH11 promoter. In various embodiments, the promoter comprises a PDGFRβ promoter, a CSPG4 promoter, and/or a SMMHC promoter. In some embodiments, specificity may be achieved or improved by combining two or three promoters (e.g., two or three of a PDGFRβ promoter, a CSPG4 promoter, and/or a SMMHC promoter).

In certain embodiments, the lower the level of NOTCH3 activity is, the greater the amount of genetic manipulation to increase NOTCH3 activity is. In various embodiments, the lower the level of NOTCH3 protein or mRNA is, the greater the amount of genetic manipulation to increase NOTCH3 expression is. In some embodiments, if a subject is heterozygous for a mutation that results in NOTCH3 loss-of-function (such as a mutation resulting in reduced function or no function), one copy of a functional NOTCH3 protein (e.g., wild-type NOTCH3) is administered (e.g., a vector is administered at a dose such that one vector enters into each cell to be genetically modified, and/or a vector comprising one open reading frame for functional NOTCH3 is administered). In certain embodiments, if a subject is homozygous for a mutation that results in NOTCH3 loss-of-function (such as a mutation resulting in reduced function or no function), two copies of a functional NOTCH3 protein (e.g., wild-type NOTCH3) is administered (e.g., a vector is administered at a dose such that two vectors enter into each cell to be genetically modified, and/or a vector comprising two open reading frames for functional NOTCH3 is administered). In various embodiments, the promoter of a vector may be selected to adjust the amount of functional NOTCH3 protein a modified cell expresses. In some embodiments, if a subject has a severe loss of NOTCH3 function, then a promoter that drives higher expression will be required. In certain embodiments, an increased dose of the vector is required to deliver a higher dose of the gene.

In various embodiments, vector comprises a natural or synthetic single or double stranded plasmid or viral nucleic acid molecule (e.g., in a viral particle) that can be transfected or transformed into a cell and replicate independently of, or within, the host cell genome. A circular double stranded plasmid can be linearized by treatment with an appropriate restriction enzyme based on the nucleotide sequence of the plasmid vector. A nucleic acid can be inserted into a vector by cutting the vector with restriction enzymes and ligating the pieces together. The nucleic acid molecule can be RNA or DNA.

In some embodiments, an embryonic stem cell (ESC), a mesenchymal stem cell, an induced pluripotent stem cell (iPSC), an iPSC-derived pericytes, or an iPSC-derived smooth muscle cell comprising an altered or exogenous NOTCH3 gene is administered to the subject.

In certain embodiments, the subject expresses NOTCH3 with any of the following CADASIL mutations: C43G, C49F, C49Y, R54C, S60C, C65S, C67Y, W71C, C76R, C76W, 77-82del, 80-84del, C87R, C87Y, R90C, C93F, C93Y, C106W, C108W, C108Y, R110C, 114-120del, C117F, S118C, C123F, C123Y, C128Y, R133C, C134W, R141C, F142C, C144S, C144Y, S145C, C146R, G149C, Y150C, 153-155del, R153C, C155S, C162S, R169C, G171C, C174F, C174R, C174Y, S180C, R182C, C183F, C183R, C183S, C185G, C185R, Y189C, C194F, C194R, C194S, C194Y, C201Y, C206Y, R207C, C212S, R213K, C222G, C222Y, C224Y, C233S, C233Y, 239-253del, C240S, C245R, C251R, Y258C, C260Y, C311G, A319C, R332C, S335C, Y337C, C349S, C379S, C395R, G420C, R421C, C428S, C428Y, C440G, C440R, C446S, R449C, C455R, C484F, C484Y, C495Y, C511R, C542Y, R544C, C549Y, R558C, R578C, R587C, R607C, C608Y, C624S, R635C, R640C, R717C, Y710C, R728C, C775S, G942C, R951C, G953C, F984C, R985C, R1006C, C1015R, Y1021C, R1031C, R1143C, D1063C, R1190C, R1201C, C1202S, R1210C, C1222G, R1231C, R1242C, C1261R, and C1261Y. In certain embodiments, the subject expresses NOTCH3 with a mutation that results in an extracellular domain of NOTCH3 having an odd number of cysteines according to the formula CnX or XnC where C stands for cysteine, n for an amino acid number in the NOTCH3 extracellular domain and X any amino replacing cysteine (for CnX) or replaced by cysteine (for XnC). In certain embodiments, n is the amino acid number (i.e., position) of any amino acid in the extracellular domain of NOTCH3. In certain embodiments, n is any one of positions 40-1643 of SEQ ID NO: 10. In certain embodiments, n is any one of positions 40-100, 100-250, 250-500, 500-750, 750-1000, 1000-1250, 1250-1500, or 1500-1643 of SEQ ID NO: 1. In certain embodiments, the subject express NOTCH3 with cysteine-sparing mutations either of the following: R61W, R75P, D80G 88-91del. See, e.g., Wollenweber et al., (2015) Cysteine-sparing CADASIL mutations in NOTCH3 show proaggregatory properties in vitro. Stroke 46(3):786-92, the entire content of which is incorporated herein by reference. In certain embodiments, the subject carries loss of function mutations in NOTCH3 including frame shift, premature stop codon, out of frame insertions or deletions, or splicing mutations including any of the following mutations: p.R113Ter, p.R103Ter, p.R156Ter, p.Y220Ter, c.1951+2delT, p.C729Ter, p.R735Ter, p.C966Ter, p.G2035RfsTer60, p.T1816ITer3, c.2566+1G>C, p.C1110Ter, p.E1125Ter, p.Y1453Ter, p.R1851VfsTer60, c.5667+1G>A, p.R1893Ter, c.5914-2_5914-linsT, p.G2035RfsTer60, p.G2035VfsTer50. See, e.g., Pippucci et al., (2015) Homozygous NOTCH3 null mutation and impaired NOTCH3 signaling in recessive early-onset arteriopathy and cavitating leukoencephalopathy. EMBO Mol Med. 7(6):848-58; Moccia et al., (2015) Hypomorphic NOTCH3 mutation in an Italian family with CADASIL features. Neurobiol Aging 36(1):547.e5-11, the entire contents of each of which are incorporated herein by reference.

In various embodiments, the SVD comprises cerebral SVD. In some embodiments, SVD comprises CADASIL. In certain embodiments, the SVD comprises CARASIL. In some embodiments, the SVD comprises diabetic retinopathy. In various embodiments, the SVD comprises cerebral SVD, CARASIL, CADASIL, age-related macular degeneration (AMD), retinopathy, nephropathy or another SVD of the kidney, microangiopathy, proximal 19p13.12 microdeletion syndrome, myocardial ischemia, heart failure, NOTCH3 loss of function-associated SVD, Alagille syndrome, familial tetralogy of Fallot, patent ductus arteriosus, a cerebral cavernous malformation, or a HTRA1-associated small vessel disease.

In some embodiments, a subject who has or is at risk of suffering from a SVD has at least 1, 2, 3, or 4 grandparents, parents, aunts, uncles, cousins, or siblings who have the SVD. In various embodiments, the subject has diabetes (e.g., type 1 diabetes or type 2 diabetes). In certain embodiments, the subject is at least about 10, 20, 30, 40, 50, 60, 70, 80, or 90 years old. In some embodiments, the subject has Alzheimer's disease. In certain embodiments, the subject has dementia. In various embodiments, the subject has arterial hypertension.

In some embodiments, the subject comprises granular osmiophilic material (GOM) deposits. In certain embodiments, the subject does not comprise GOM deposits.

In various embodiments, a subject who has or is at risk of suffering from a SVD has an abnormal level of NOTCH3, collagen18α1 or endostatin, IGFBP-1, and/or HTRA1 protein or mRNA. In some embodiments, a test sample obtained from the subject comprises a level of NOTCH3 protein or mRNA that is different than a normal control. For example, the test sample may comprise a level of NOTCH3 protein or mRNA that is at least about 5%, 10%, 15%, 20%, 25%, 30%, 40%, 45%, 50%, 55%, 60%, 65%, 70%, 75%, 80%, 90%, 95%, 99%, 100%, 5-50%, 50-75%, or 75-100% lower in the test sample compared to a normal control. In some embodiments, the test sample comprises a level of collagen18α1 or endostatin and/or HTRA1 protein or mRNA that is at least about 5%, 10%, 15%, 20%, 25%, 30%, 40%, 45%, 50%, 55%, 60%, 65%, 70%, 75%, 80%, 90%, 5-50%, 50-75%, 1-fold, 2-fold, 3-fold, 4-fold, or 5-fold higher in the test sample compared to a normal control. In certain embodiments, a test sample comprises a level of HTRA1 protein or mRNA that is at least about 5%, 10%, 15%, 20%, 25%, 30%, 40%, 45%, 50%, 55%, 60%, 65%, 70%, 75%, 80%, 90%, 95%, 99%, 100%, 5-50%, 50-75%, or 75-100% higher in the test sample compared to a normal control. In certain embodiments, a test sample comprises a level of HTRA1 protein or mRNA that is at least about 5%, 10%, 15%, 20%, 25%, 30%, 40%, 45%, 50%, 55%, 60%, 65%, 70%, 75%, 80%, 90%, 95%, 99%, 100%, 5-50%, 50-75%, or 75-100% lower in the test sample compared to a normal control. In some embodiments, the subject (e.g., a test sample from the subject) comprises a level of NOTCH3 protein bound to collagen18α1 and/or endostatin and/or HTRA1 and/or IGFBP-1 that is different than a normal control. For example, the test sample may comprise levels of NOTCH3 protein bound to collagen18α1 and/or endostatin and/or HTRA1 and/or IGFBP-1 that is at least about 5%, 10%, 15%, 20%, 25%, 30%, 40%, 45%, 50%, 55%, 60%, 65%, 70%, 75%, 80%, 90%, 5-50%, 50-75%, 1-fold, 2-fold, 3-fold, 4-fold, or 5-fold higher in the test sample compared to a normal control. Non-limiting examples of test samples include blood, serum, plasma, saliva, tears, vitreous, cerebrospinal fluid, sweat, cerebrospinal fluid, or urine.

In various embodiments, a test sample comprises a level of HTRA1 protein or mRNA that is at least about 5%, 10%, 15%, 20%, 25%, 30%, 40%, 45%, 50%, 55%, 60%, 65%, 70%, 75%, 80%, 90%, 95%, 99%, 100%, 5-50%, 50-75%, or 75-100% higher in the test sample compared to a normal control. In some embodiments, the subject has or is at risk of suffering from CADASIL.

In various embodiments, a test sample comprises a level of HTRA1 protein or mRNA that is at least about 5%, 10%, 15%, 20%, 25%, 30%, 40%, 45%, 50%, 55%, 60%, 65%, 70%, 75%, 80%, 90%, 95%, 99%, 100%, 5-50%, 50-75%, or 75-100% lower in the test sample compared to a normal control. In some embodiments, the subject has or is at risk of suffering from CARASIL.

Aspects also provide a composition comprising an effective amount of a vector comprising a genetic construct and an ophthalmically acceptable vehicle, wherein the genetic construct comprises a coding sequence that encodes NOTCH3 and a promoter, wherein the coding sequence is operably linked to the promoter. In some embodiments, the vector comprises a plasmid. In some embodiments, the vector comprises a viral vector. In certain embodiments, the composition is in the form of an aqueous solution comprising an osmolality of about 200 to about 400 milliosmoles/kilogram water.

Also included is a non-viral vector for treating or preventing a SVD in a subject, wherein the non-viral vector comprises a genetic construct, wherein the genetic construct comprises a coding sequence that encodes NOTCH3 and a promoter, wherein the coding sequence is operably linked to the promoter.

The present subject matter further provides a viral vector for treating or preventing a SVD in a subject, wherein the viral vector comprises a genetic construct that comprises a promoter and a coding sequence that encodes NOTCH3, wherein the coding sequence is operably linked to the promoter.

Also included is the use of a genetic construct in the manufacture of a medicament for treating or preventing a SVD in a subject, wherein the genetic construct comprises a coding sequence that encodes NOTCH3 and a promoter, wherein the coding sequence is operably linked to the promoter.

Each embodiment disclosed herein is contemplated as being applicable to each of the other disclosed embodiments. Thus, all combinations of the various elements described herein are within the scope of the invention.

Other features and advantages of the invention will be apparent from the following description of the preferred embodiments thereof, and from the claims. Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. GenBank or other sequence database references as well as the contents of journal articles or other publications are hereby incorporated in their entirety by reference.

### DESCRIPTION OF THE DRAWINGS

FIG. 1A is a cartoon, FIG. 1C is a set of graphs and FIGS. 1D, E, F, and G are images showing human NOTCH3 rescue of mural cell loss in N3KO mice. (FIG. 1A) Schematic representation of four mouse strains utilized to study genetic rescue of NOTCH3 signaling: wild-type NOTCH3 (N3WT, white), NOTCH3 knockout (N3KO, light gray), N3KO mice conditionally expressing wild-type human NOTCH3 (hN3WT, dark gray) and N3KO mice conditionally expressing a human CADASIL mutant NOTCH3 (C455R, black). (FIG. 1B) Representative immunofluorescence images of retinal whole mounts showing SMA staining in red and collagen IV (ColIV) in green (left, scale bar-2.5 mm). Red dashed rectangles in left panels indicate regions displayed in center panels. (right, scale bar=250 μm) (FIG. 1C) Quantification of SMA coverage in main retinal arteries and branching arterioles. n=5 for each group, *p<0.05,**p<0.01, statistical analysis was done via ANOVA. (FIG. 1D) Representative ultra structural images of retinal vessels (scale bar=200 μm) and cerebral vessels from the left hemisphere of the cerebral cortex cut at the bregma, (scale bar=2 μm) (FIG. 1E) obtained by transmission electron microscopy. Lumen (Lu), vascular endothelial cell (EC), basement membrane (BM), mural cell (MC), gaps in MC (black arrows) and apoptotic bodies (white arrows). Similarly, six features, listed above are highlighted on each image from retina (FIG. 1F) and brain (FIG. 1G). The N3WT mice exhibit large block-like MC that are in contact or are closely associated whereas the N3KO and C455R mice exhibit large gaps and elongated MCs. hN3WT exhibits elongated MCs juxtaposed to each other. Scale bar=1 μm.

FIG. 2 is a set of images showing image processing of vessels via FIJI-based macro. Images of retinal whole mounts stained with collagen IV (Col IV) in green and smooth muscle actin (SMA) in red were processed. Seven images tracing a single vessel from optic nerve to periphery were stitched together using FIJI's MosaicJ macro. This was done for three vessels per retina/animal. The vascular analysis macro generates an outline of the vascularized area based on the Col IV silhouette, and is then cut up into small rectangles, each of which is identified as part of the main vessel, shown as green rectangles, or as part of branching vessels, shown as blue rectangles. The squares are then superimposed onto the red, SMA binary image and determined to have or not to have SMA staining. Rectangles containing a value of 0, having no SMA staining are qualified as gaps. The SMA positive areas are analyzed and qualified as main vessel coverage, shown as red outlined areas, or branching vessels, shown as orange outlined areas. The macro then saves parameters for each of the vessel types as an excel spreadsheet. Scale bar=200 μm

FIG. 3A are images of fluorescein angiography (FA) of the retina, FIG. 3B is a graph relating to leakage events. (FIG. 3A) Representative FA images from wild-type NOTCH3 (N3WT), NOTCH3 knockout (N3KO), N3KO mice conditionally expressing wild-type human NOTCH3 (hN3WT), N3KO mice conditionally expressing a human CADASIL mutant NOTCH3 (C455R), and N3KO mice conditionally expressing a human CADASIL mutant NOTCH3 and a wild-type human NOTCH3 (C455R/hN3WT). Arrows indicate leakage events. (FIG. 3B) Graph shows quantification of leakage events in the listed genotypes: N3WT (n=6), N3KO (n=6), hN3WT (n=5), C455R (n=3), C455R/hN3WT (n=5). ** p<0.05. NS means non significant.

### DETAILED DESCRIPTION

Provided are methods and compositions for treating SVD, including CADASIL and NOTCH3 loss of function-associated SVD, via gene therapy. Cerebral SVD affects about a third of individuals over 80 years of age, and is a leading cause of stroke, cognitive impairment, and dementia. No disease modifying therapies are available and most treatments focus on managing cardiovascular risk factors known to contribute to the disease. Loss of mural cells, which encompass pericytes and vascular smooth muscle cells, is a hallmark of SVD resulting in vascular instability. NOTCH3 signaling is both necessary and sufficient to support mural cell coverage in arteries using genetic rescue, and SVD may be treated by modulating NOTCH3 signaling.

Small vessel diseases are highly prevalent and impact highly vascularized tissues such as the brain, retina, and the kidney. In the brain, small vessel disease is the most prevalent neurological condition and a strong contributor to the susceptibility to stroke, vascular cognitive impairment, and dementia. In the retina, small vessel disease plays a critical role in early stage diabetic retinopathy, which is characterized by mural cell loss. Prior to the methods and compositions provided herein, there were no specific treatments to prevent mural cell degeneration in small vessel disease. Included herein are methods of treating SVD with Notch signaling activators, including gene therapy constructs and gene replacement. In some embodiments, mural cell degeneration is reduced or prevented in SVD, e.g., in vascularized tissues such as retina and brain.

CADASIL is a monogenic cause of cerebral small vessel disease associated with mutations in the NOTCH3 gene. There are no specific treatments for small vessels disease in general or CADASIL in particular. Methods and compositions provided herein solve this practical problem by using NOTCH3 signaling in mural cells as a therapeutic target to prevent mural cell loss in small vessel diseases. Prior to the present invention, there were no methods to address mural cell loss in SVD.

NOTCH3 loss of function-associated SVD is distinct from CADASIL, because it lacks the characteristic accumulation of NOTCH3 extracellular domain in vessels and it lacks granular osmiophilic deposits (GOMs). Prior to the present invention, there were no methods to address mural cell loss in NOTCH3 loss of function-associated SVD. In embodiments, a subject with such a SVD has symptoms that are similar to CADASIL. See, e.g., Moccia et al., (2015) Hypomorphic NOTCH3 mutation in an Italian family with CADASIL features. Neurobiol Aging 36(1):547.e5-11; and Fouillade et al., (2008) Activating NOTCH3 mutation in a patient with small-vessel-disease of the brain. Hum Mutat. 29(3):452, the entire contents of each of which are incorporated herein by reference. In various embodiments, the subject has migraine headaches. In some embodiments, the subject has migraines with aura. In certain embodiments, the skin of the subject comprises vascular damage. In various embodiments, the subject has cerebral SVD. A non-limiting example of a mutation that may result in a SVD symptoms similar to CADASIL is a C to U substitution at position 307 of the open reading frame of NOTCH3-encoding mRNA (a cDNA sequence is provided as SEQ ID NO: 2), which results in a truncation of the protein such that amino acids from position R103 to the wild-type C-terminus are missing.

In some embodiments, the subject comprises a R103X substitution mutation. In certain embodiments, the mutation results in a substitution or a truncation within an Epidermal Growth-Factor-like Repeat of NOTCH3. In various embodiments, the mutation is within one of exons 2-24 of the NOTCH3 gene. In some embodiments, the mutation is a missense mutation in exon 25 of NOTCH3. In certain embodiments, the mutation comprises a substitution or mutation within the heterodimerization domain of Notch. In various embodiments, the mutation results in a L1515P substitution. In some embodiments, the substitution is not a conservative substitution. In certain embodiments, the subject comprises an autosomal mutation in NOTCH3. In some embodiments, the mutation results in reduced NOTCH3 function. In various embodiments, the mutation results in increased NOTCH3 function. In some embodiments, the mutation comprises substitution in the extracellular domain of NOTCH3 that adds or removes a cysteine compared to wild-type NOTCH3. In certain embodiments, the mutation comprises a truncation beginning in the extracellular domain of the NOTCH3 protein. In embodiments, the subject is heterozygous for the mutation. In embodiments, the subject is homozygous for the mutation.

Without being bound by any scientific theory, the non-limiting data herein show for the first time that NOTCH3 signaling is both necessary and sufficient to sustain mural cell coverage in arteries via a cell autonomous effect. This finding is demonstrated, e.g. by rescuing mural degeneration in a NOTCH3 knockout by expressing the human NOTCH3 protein specifically in mural cells. This finding is demonstrated, e.g. by rescuing vascular leakage events in a NOTCH3 knockout by expressing the human NOTCH3 protein specifically in mural cells. This finding is demonstrated, e.g. by rescuing vascular leakage events in a NOTCH3 knockout expressing the C455R CADASIL mutation by also expressing the wild-type human NOTCH3 protein specifically in mural cells. This finding is demonstrated, e.g. by the observation of indistinguishable levels of mural cell loss and frequency of vascular leakage events between NOTCH3 knockouts and NOTCH3 knockouts expressing the C455R CADASIL mutation. This finding shows that a gene replacement approach, in which a defective NOTCH3 is replaced by a wild-type NOTCH3 specifically in mural cells, results in a functional rescue. This is surprising because NOTCH3 is expressed in other cell types including monocytes/macrophages, other immune cells, and stem cells all of which have been shown to play roles in the vasculatures. See, e.g., Fung et al., (2007) Delta-like 4 induces notch signaling in macrophages: implications for inflammation. Circulation 115(23):2948-56; and Lafkas et al., (2013) Notch3 marks clonogenic mammary luminal progenitor cells in vivo. J Cell Biol. 203(1):47-56, the entire contents of which are hereby incorporated herein by reference. This is surprising because the predominant view is that CADASIL mutations in NOTCH3 operate via neomorphic toxic gain of function effects. It is therefore unexpected and surprising that by increasing NOTCH3 signaling in mural cells one can rescue vascular degeneration and thus bypass the neomorphic effects.

The following findings were unexpected:
(i) The discovery was made using a NOTCH3 mutation associated with the human small vessel disease called CADASIL (cerebral autosomal dominant arteriopathy with subcortical infarcts and leukoencephalopathy) (Arboleda-Velasquez et al., Neurology, 2002; Arboleda-Velasquez et al., PNAS, 2008; Arboleda-Velasquez et al., PNAS, 2011). In that condition, the most widely accepted pathobiological mechanism is that of toxic neomorphism via accumulation of the NOTCH3 receptor extracellular domain whereas NOTCH3 signaling defects due to the mutations are not considered to be primary drivers of the disease (Joutel et al., J Cereb Blood Flow Metab, 2016). In a challenge to this paradigm, the data herein shows that mural cell loss and vascular leakage in a model carrying the CADASIL mutation was similar to that of the mice lacking NOTCH3 (knockout), suggesting a negligible contribution of the toxic neomorphic effects. It is also surprising that introducing a wild-type Notch3 copy was able to rescue an SVD phenotype in mice expressing a Notch3 CADASIL mutant receptor, suggesting again a negligible contribution of the toxic neomorphic effects.
(ii) The discovery was made using a NOTCH3 knockout mouse model. NOTCH3 loss of function mutations including premature stop codons and frame shifts have been reported in individuals with SVD. See, e.g., Pippucci et al., (2015) Homozygous NOTCH3 null mutation and impaired NOTCH3 signaling in recessive early-onset arteriopathy and cavitating leukoencephalopathy. EMBO Mol Med. 7(6):848-58; Moccia et al., (2015) Hypomorphic NOTCH3 mutation in an Italian family with CADASIL features. Neurobiol Aging 36(1):547.e5-11, the entire contents of each of which are incorporated herein by reference. However, the clinical significance of these mutations has been highly debated and the most widely accepted view is that they represent variants of polymorphic nature. See, e.g., Rutten et al., (2013) Hypomorphic NOTCH3 alleles do not cause CADASIL in humans. Hum Mutat. 34(11):1486-9, the entire content of which is incorporated herein by reference. In a challenge to this paradigm, the data herein shows that a NOTCH3 knockout develops an SVD phenotype that can be rescued by expression of wild-type NOTCH3 in mural cells, unambiguously linking NOTCH3 deficiency to SVD in an experimental model.
(iii) Because Notch signaling regulates a developmental program, it is unexpected that a process resulting from defective signaling could be corrected or prevented from being fulfilled. Accordingly, the ability of gene therapy that reestablishes physiological (e.g., by increasing or decreasing NOTCH3 function) expression or activity of NOTCH3 to prevent or treat mural cell loss is surprising.
(iv) Because NOTCH3 is expressed in other cell types that influence the vasculature, it is unexpected that vascular defects could be rescued by expressing NOTCH3 only in mural cells.

In embodiments, vascular defects can be rescued by expressing NOTCH3 only in perivascular fibroblast-like cells. See, e.g., Vanlandewijck et al. (2018) “A molecular atlas of cell types and zonation in the brain vasculature” Nature volume 554, pages 475-480, the entire content of which is incorporated herein by reference. In various embodiments, a tissue-specific or cell type-specific is used to direct the expression of NOTCH3. In some embodiments, the promoter directs expression in a specific cell type such as mural cells (e.g., pericytes and vascular smooth muscle cells). In certain embodiments, the promoter directs expression in pericytes (e.g., the promoter is a desmin, a NOTCH3, an alpha-smooth muscle actin, a PDGFRβ, or a CSPG4 promoter). In various embodiments, the promoter directs expression in vascular smooth muscle cells (e.g., the promoter is a SM22 promoter). In some embodiments, the promoter directs expression in endothelial cells (e.g., the promoter is a Tie2, Fli-1, vascular endothelial-cadherin, endoglin, Flt-1, or an intercellular adhesion molecule 2 promoter. In various embodiments, some of these promoters may need to be combined to achieve cell type specificity. In various embodiments, the promoter comprises a desmin or an alpha-smooth muscle actin (α-SMA) promoter. In some embodiments, the promoter comprises a SM22 promoter. In certain embodiments, the promoter comprises a SM22α promoter. In various embodiments, the promoter comprises a Tie2, Fli-1, vascular endothelial-cadherin (VE-cadherin), endoglin, Flt-1, or intercellular adhesion molecule 2 promoter (ICAM-2) promoter. In certain embodiments, the promoter is specific for smooth muscle cells and pericytes [e.g., a NOTCH3 promoter or a platelet-derived growth factor receptor beta gene (PDGFRβ) promoter]. In some embodiments, the promoter is a NOTCH3 promoter. In embodiments, the promoter is a PDGFRβ promoter. In certain embodiments, the construct is administered to the subject in a liposome. In some embodiments, a non-viral vector (e.g., a plasmid that comprises a construct) is combined with an agent that facilitates its entry into cells such as a condensing agent. In some embodiments, the vector is condensed with an agent such as PEI, PLL, a PAMAM dendrimer, spermidine, spermine, or cobalthexamine. In some embodiments, a MNP that comprises the vector is administered to the subject. In certain embodiments, the promoter comprises a MYH11 promoter. In various embodiments, the promoter comprises a PDGFRβ promoter, a CSPG4 promoter, and/or a SMMHC promoter. In some embodiments, specificity may be achieved or improved by combining two or three promoters (e.g., two or three of a PDGFRβ promoter, a CSPG4 promoter, and/or a SMMHC promoter).

In various embodiments, NOTCH3 binding to itself or another protein (such as collagen18α1/endostatin, IGFBP-1, and/or HTRA1) is indicative of SVD or a risk of developing a SVD. See, e.g., Arboleda-Velasquez et al. (2005) “CADASIL mutations impair Notch3 glycosylation by Fringe” Hum Mol Genet. 14(12):1631-9, the entire contents of which are incorporated herein by reference. In some embodiments, a NOTCH3 homodimer is indicative of SVD or a risk thereof. In certain embodiments, a NOTCH heterodimer is indicative of SVD or a risk thereof. In various embodiments, the protein-protein interaction is an aberrant protein-protein interaction. In certain embodiments, the method further comprises diagnosing a subject as having an SVD (such as CADASIL) if NOTCH3 is binding to collagen18α1/endostatin, IGFBP-1, and/or HTRA1 is detected in the subject (e.g., in a test sample from the subject).

In various embodiments, a mutation in the NOTCH3 gene triggers adult-onset stroke and vascular dementia in, e.g., a subject with a SVD such as CADASIL. In some embodiments, the mutation affects an epidermal growth factor-like (EGF-like) repeat located in the extracellular domain of the NOTCH3 receptor. EGF-like repeats in the NOCTH3 receptor is also the target of sequential complex O-linked glycosylation mediated by protein O-fucosyltransferase 1 and Fringe. In certain embodiments, the mutation does not affect the addition of O-fucose but does impair carbohydrate chain elongation by Fringe. In various embodiments, a subject has aberrant homodimerization of mutant NOTCH3 fragments and/or heterodimerization of mutant NOTCH3 with Lunatic Fringe itself. In certain embodiments, a subject has a complex (such as a dimer) comprising N3ECD and at least one other protein. In various embodiments, the interaction between the components of a homodimer or heterodimer comprising NOTCH3 or a portion thereof (e.g., a mutant NOTCH3 or N3ECD) is enhanced by one or more abnormal disulfide bonds. In some embodiments, a NOTCH3 homodimer comprises one or more disulfide bonds covalently connecting each NOTCH3 monomer.

In various embodiments, a subject comprises a N3ECD homodimer. In some embodiments, the N3ECD homodimer comprises one or more disulfide bonds covalently connecting each NOTCH3 monomer.

### Exemplary Promoter Sequences

A promoter sequence for human NOTCH3 is as follows:

(SEQ ID NO: 13)
AAATGTAGTCCTAGGCTCCTGGCCTGGCTGAGTGAGTTTTAGCGGTTTT
TTGTTTGTTTGTTTTGTTTGAGACAGGGTCTTACTCTGTTGCCCAGGCT
GGAGCGCAGTGGTACGATTTCGGCTAAGTGCAACCTCAGCCTCCTGGGT
TGAAGCGATTTTCCTGCTTCAGCCTCTCGAATAGCTGGGACTACAGGCG
TGCGCCACCACGCTCAGCTAATTTTTGTATTTTCAGTAGAGTCCGGGTT
TCACCATGTTGGCCAGGCTGGTCTCCAACTCTTGACCTCAAGTGATCCA
TCCACCTCTGCCTCTCAAAGTGCTGGGATTACAGGCCGGAGCCACCGCG
CCCGGTCCGACTTTTAGGTTTGTGAATGTTTTAGATCAGAGTCCTGGGG
AAGCTTGGTGCATGGGCTTGCATTTGTGCGTCCGTGGCTGTGGGTCCAT
GAGCCTCTCAGGACGTGACTGGCCTCAGTTTCCAGAGTTTCTGGGAGGC
TGTGTTTTTTGTCCCGGCTCCAGAGGTGTCCGGCTCTGGGTGTGTACTG
GGGGATGGGGATGGGGTGCGTGGGCGTTCACGAGGTTGGGTGTGCCCGC
CACTCCGGGTTCTGCCCGCGTCTCACTGCATGCTCGGCCTGGGTTTCCG
AGGGTCCGCGCGTCCCAGGCTGTGCGGGTGGAGGGTGGGCAGGGACCCC
GGGAGGCCGGGCGGGGGGCGGGGGCGCGCTGGGCCGGCCCCGGGGCGGG
GCGAGCCTTCGAGGGCTGGGGGCGGGGCGGCCCGGCCGCCTCACTTCGG
CGAAGTTGGCGGCGCGGAGGCTGGCCCGGGACGCGCCCGGAGCCCAGGG
AAGGAGGGAGGAGGGGAGGGTCGCGGCCGGCCGCCATGGGGCCGGGGGC
CCGTGGCCGCCGCCGCCGCCGTCGCCCGATGTCGCCGCCACCGCCACCG
CCACCCGTGCGGGCGCTGCCCCTGCTGCTGCTGCTAGCGGGGCCGGGGG
CTGCAGGTGAGGGGCCGGGACCTGGCGGATGGGACGAGGGCGGCAGAGG
GGGAGTGCAAGAAC

A promoter sequence for human platelet derived growth factor receptor beta is as follows:

(SEQ ID NO: 14)
GCTTTGAGACGTGAAAAGGAATGTATCTGTCAATATCCAGAGATATCCA
ATCTGTGCTGGATGACCTTGACCAAATCACATTCCCTCTCTGTGCCAAT
TCACCCCTGGGCCTTGGTTTGTCCTTCTGAAAAACGGCTGATCATCTTG
GAGGATCTTTCCATCTCCTCCATGTCACCTCTTCTCAGCAGTGCTCTCT
GATTCTCTCAGGCTTCCGTTAGGGGTCCTTCTAAATGCACTCTCAGCCT
CCTGTCTGCCGACCGGGCACCTGCTTGCCCAATGAATATCTTGCTCACT
AGCCTGGTGCTTCTGAGGTCAGGACCACGTCTTGCTACTGTCCCCTGCA
CCCAGTACAGTGTTAGAATGTAGTAAATGCTCAGTATGCTTGTTCAATG
AATGAAGGTTGACTGAATCAACCACAGAGATGATCAGAGTCTCCTTTAC
AGATGGGGAAACTGAGGCACTGAGAAATGATCTCCCTGGGTGCCATGGC
AAGGGGAGGCCGCCACCCGACATGGGCATCGGGAAAGTGGGGCCGAGGC
CACTCTTTGACAAAAGTGACTCAGCAGCTGGCGGCTGGCGTGGGCTGTG
GAATTTCTAAGCATCCCCTCACATCCTGAGCGAACGGGCGATGGGAGGG
AGTAAGCAGAGTGAAAAGAAGAAAAACAAGAAACTTGGGGGAAAAGAAA
GAGAGAGGGGGAAAAAAACTGAAATTGAAAACAGACGCACGCGTCCACC
CTCCCTGCCCCGCCGCCCCCCCTTCTCCCAGCTGGGCCAATCAGAAGCC
ACCCTGCAGCCTTTCCCCCAAAGTGGCGGGGGAAGTGGGGGGGCATGGA
AGCCCATAGTGGTGTGAGCTCCTGAGGCTGCCAGCAGCCAGCAGTGACT
GCCCGCCCTATCTGGGACCCAGGATCGCTCTGTGAGCAACTTGGAGCCA
GAGAGGAGATCAACAAGGAGGAGGAGAGAGCCGGCCCCTCAGCCCTGCT
GCCCAGCAGCAGCCTGTGCTCGCCCTGCCCAACGCAGACAGCCAGACCC
AGGGCGGCCCCTCTGGCGGCTCTGCTCCTCCCGAAGGAT

A promoter sequence for human transgelin is as follows:

(SEQ ID NO: 15)
GCCAGGCCTTATAGACCCTCTTATTGACACTGCCCACTGGATGGGGACC
GGAGTTGGACTCAGCTTCTGCCGAACCCTCAAATCCCAGCCCCAACTAA
AGCATATAACTCAAGACCTACCTGCACTGAAAGCTCTTCTCAACCTGAG
CAGGGTGGTCCAATTGAAAGGGTGGGTCTGACCACCTCTCCTGCACCCA
TGCGGGTTGGCAGAGGTGTGCAGGATCTGCCACTTACCATTCACCATGT
GGCCTTGAGGAAGACGCACTCGGGGCCTCAGTTTCCTCATCTATAAAAT
GGGGATGTAATTACACCCTCACACTGTAGCTGTGAGTATTCAATGAGAG
CACTGCAAAGGGCCTGGTGTGGAGTAGGTCCTCAGGAAAGGTTGGATCC
CATGTCCCATCAGAGCTAAAAGCCCCAGGAGGAGAGGGTGGCTGGTTTG
TCCCCACAAACCCCTGGGATTCCCGGCTCCCCAGCCCCTTGCCCCTCTC
TCCAGCCAGACTCTATTGAACTCCCCCTCTTCTCAAACTCGGGGCCAGA
GAACAGTGAAGTAGGAGCAGCCGTAAGTCCGGGCAGGGTCCTGTCCATA
AAAGGCTTTTCCCGGGCCGGCTCCCCGCCGGCAGCGTGCCCCGCCCCGG
CCCGCTCCATCTCCAAAGCATGCAGAGAATGTCTCGGCAGCCCCGGTAG
ACTGCTCCAACTTGGTGTCTTTCCCCAAATATGGAGCCTGTGTGGAGTC
ACTGGGGGAGCCGGGGGTGGGGAGCGGAGCCGGCTTCCTCTAGCAGGGA
GGGGGCCGAGGAGCGAGCCAGTGGGGGAGGCTGACATCACCACGGCGGC
AGCCCTTTAAACCCCTCACCCAGCCAGCGCCCCATCCTGTCTGTCCGAA
CCCAGACACAAGTCTTCACTCCTTCCTGCGAGCCCTGAGGAAGCCTTGT
GAGTGCATT

A promoter sequence for human CSPG4 is as follows:

(SEQ ID NO: 16)
AGTAACATTGCCCTTTCTCCTCCTGTTTGGGACTAGGCTGATGTTGCTG
CCTGGAAGGGAGCCAGCAGAAGGGCCCCAGCCTGAAGCTGTTAGGTAGA
AGCCAAATCCAGGGCCAGATTTCCAGGAGGCAGCCTCGGGAAGTTGAAA
CACCCGGATTCAGGGGTCAGGAGGCCTGGGCTTCTGGCACCAAACGGCC
AGGGACCTACTTTCCACCTGGAGTCTTGTAAGAGCCACTTTCAGCTTGA
GCTGCACTTTCGTCCTCCATGAAATGGGGGAGGGGATGCTCCTCACCCA
CCTTGCAAGGTTATTTTGAGGCAAATGTCATGGCGGGACTGAGAATTCT
TCTGCCCTGCGAGGAAATCCAGACATCTCTCCCTTACAGACAGGGAGAC
TGAGGTGAGGCCCTTCCAGGCAGAGAAGGTCACTGTTGCAGCCATGGGC
AGTGCCCCACAGGACCTCGGGTGGTGCCTCTGGAGTCTGGAGAAGTTCC
TAGGGGACCTCCGAGGCAAAGCAGCCCAAAAGCCGCCTGTGAGGGTGGC
TGGTGTCTGTCCTTCCTCCTAAGGCTGGAGTGTGCCTGTGGAGGGGTCT
CCTGAACTCCCGCAAAGGCAGAAAGGAGGGAAGTAGGGGCTGGGACAGT
TCATGCCTCCTCCCTGAGGGGGTCTCCCGGGCTCGGCTCTTGGGGCCAG
AGTTCAGGGTGTCTGGGCCTCTCTATGACTTTGTTCTAAGTCTTTAGGG
TGGGGCTGGGGTCTGGCCCAGCTGCAAGGGCCCCCTCACCCCTGCCCCA
GAGAGGAACAGCCCCGCACGGGCCCTTTAAGAAGGTTGAGGGTGGGGGC
AGGTGGGGGAGTCCAAGCCTGAAACCCGAGCGGGCGCGCGGGTCTGCGC
CTGCCCCGCCCCCGGAGTTAAGTGCGCGGACACCCGGAGCCGGCCCGCG
CCCAGGAGCAGAGCCGCGCTCGCTCCACTCAGCTCCCAGCTCCCAGGAC
TCCGCTGGCTCCTCGCAAGTCCTGCCGCCCAGCCCGCCGGGATGCAGTC
CGGGCCGCGGCCCCCACTTCCAGCCCCCGGCCTGGCCTTGGCTTTGACC
CTGACTATGTT

A promoter sequence for human RGS5 is as follows:

(SEQ ID NO: 17)
CCTTGAGTGATAGGAAGCAGGAAAAGTTTGAAGGAGAAGGAAAGACAAG
CCTAGGCAAGGATGGCTATGGTGGGGAGGTACAAAGAATTACAAAGCAT
TAGTCATCCAAGCAAAGTATAATCAGAAGACCACTGACAGAATTAACAG
AGTTCAGAGATAATATCATTATACTCAATCCCTACTAAGATTTTGGTGT
TTGGTCTCCTAGATCACTTTTTTAGTACCTTCACAGACATATGGATTCA
TAAAGAATATATAGTAATTATTCATATTTGTTTTGATTTATATAAACAG
TAATCTTGTCTCTCAGCTCCAGAAAACCTTGCCTGGTGCTTCCTGAAGG
CTCACAACATCCCAGCCCGGCCTCCATCACTTACTTATAATGTGTTTTG
GGGTCTTGAGGGCAGGGAGCTTTCTCTTTCTTCTCTGAATTCCTAGCAT
CCAGCTGAGTGCCTGGCACTTGGTAAGTATTCACATACACATTCCTTGA
ATTCCCTAGATTTCAGTTTCCTTCACTGTGAAACAAAGTGGGCTAAACG
ATCTCCAGGAGCCCTTACAGTCCTAAGAATCTATGACTTTGACCGATCT
GAAGTGGTCCAAGACACTTTGCTTCACAGGAAGCTCACATCTTCCTTGT
GATTGGCCTGAGATTAGGAGTTCAAACAACCAATGACCTGTGCACAGTT
TAGGGCCTAACCCTGCCCTGGCCGGTTAGCAAGAGAGTGTTGTGATAAG
AGCAGATGAAAGCCTTATGCCACAGTAGTGCCTGTAGCAGAGAAAGGAT
TCCCTCCTCCACCTCAGGGCCCACTGCTGCTGCTCTCCAGCCTGCAGTT
TCCTCTAAGGCTCTGGATTGGCTGGAAGAGCAACAGAGGGCTGGGAAAG
AGCTTCTATATATACCTCAGGAGGAAAGGCATCCCAGACAGTTTTGAAG
TTTTCAAAGACTGGCTCTGCTGTTAAGAAGTTGTACTTAAAGCGGAGGA
GCTAAGCCACCTGCCAAAATGTGCAAAGGACTTGCAGCTTTGCCCCACT
CATGCCTGGAAAGGTGAGAACCAAGTT

A promoter sequence for human MYH11 is as follows:

(SEQ ID NO: 18)
AGGGAAGTTGTTGAAGGGAACTCGCAGACTCTGGG
GGCACACCCACTTTCTCCTTGGATCTTGACACTTG
CATCTTGTAAATAACGTAATTATCACCGCCACCGC
CTTCCCCCATTTTGTAGCTATGGACACCAAGTCTC
AGAGAAGTGAAGTGACTTGCCCAAGGTCACGCAGC
TGGCGAGTGGCGCACAGGGGAGGGGGACAGCTGAA
ATAATCACAGTGGGCTTATTTTTAATTTTTATTTG
TATTTTGGTCGTGGTGATGTGGGTGGAGGTGGAGA
TGGCAAGTTGGGAAAAGTAAAAACTTCCCCTTCCT
GCACGGTTCCCAGCAAGGGTGGGGGCCTCCTGTCT
TGCACTTTGCAAAGTTCAAGAAATCCCCTTTCCCT
ACCCTTCACGCTGCACAGCCGGCCCTCTTTCCAGA
CAGTGCGATGCCAATAAAATGGGAAGTGGGGTGGG
AGATGTCAAGTCAGATCCACCACAGCCCCGACACG
GGGAGGAAGAGGTTAAAGCCTTTGCGGCCGGAACC
GACTCAGGGAAGACGTTCTCAAGCATCCCGCACAG
ACACTGCCTGCTCGACCCCCTTTCTCTAGGGATCC
GGAGCGTCTGCGACCGCCTGGGGCCGGGGCTGAGA
CTCCCGTCCCTGTGCGCACCTGTTCCGTGCGCCCT
TGTGCGGTGCGCACCTGTTCCGTGCACCCTTGTCC
CGAGCGCCCCAGCTCCTTGCGCTCCCGCCGGGGGT
GCGCCCTGCAGGGGGCGCGGCGAGGGGGCCGCGAG
GGACCCTCCCCAACTCCACCCCTTCGGCCTCCTCC
CCTTTCCCAGCCGCGGGCAGCTCCGGGTCTATAAA
GAGAGGCGTCCGAGGACGCGCAGGGAGATTTGGAC
GCTCCGGCCTGGGAGGTGCGTCAGATCCGAGCTCG
CCATCCAGTTTCCTCTCCACTAGTCCC

### NOTCH3

The NOTCH3 gene encodes the third discovered human homologue of the Drosophila melanogaster type I membrane protein notch. In Drosophila, notch's interaction with its cell-bound ligands (delta, serrate) establishes an intercellular signaling pathway that plays a key role in neural development. Homologues of the notch-ligands have also been identified in humans, but precise interactions between these ligands and the human notch homologues remains to be determined. NOTCH3 functions as a receptor for membrane-bound ligands JAGGED1, JAGGED2 and DELTA1 to regulate cell-fate determination. NOTCH3 has been proposed to affect the implementation of differentiation, proliferation and apoptotic programs. Mutations in NOTCH3 have been identified as the underlying cause of cerebral autosomal dominant arteriopathy with subcortical infarcts and leukoencephalopathy (CADASIL) and other SVD conditions similar to CADASIL.

The cytogenetic band of the NOTCH3 gene has been reported to be 19p13.12 by Ensembl, 19p113.12 by Entrez Gene, and 19p13.12 by the HUGO Gene Nomenclature Committee. According to Ensemble, the location of the NOTCH3 gene is Chromosome 19: 15,159,038-15,200,981 reverse strand (Ensembl release 87—December 2016). Additionally, this gene maps to 15,269,849-15,311,792 in GRCh37 coordinates. Non-limiting examples of NOTCH3 genomic sequences are available from public databases such as Genbank (see, e.g., Accession Nos. NC_000019.10 and AH006054.2).

An amino acid sequence for human NOTCH3 is publically available in the UniProt database under accession number Q9UM47 (SEQ ID NO: 1) and is as follows (exemplary sites that may be substituted in subjects with SVD including CADASIL, cysteine-sparing mutations and NOTCH3 loss of function mutations are bolded and underlined):

MGPGARGRRRRRRPMSPPPPPPPVRALPLLLLLAG
PGAAAPPCLDGSPCANGGRCTQLPSREAACLCPPG
WVGERCQLEDPCHSGPCAGRGVCQSSVVAGTARFS
CRCPRGFRGPDCSLPDPCLSSPCAHGARCSVGPDG
RFLCSCPPGYQGRSCRSDVDECRVGEPCRHGGTCL
NTPGSFRCQCPAGYTGPLCENPAVPCAPSPCRNGG
TCRQSGDLTYDCACLPGFEGQNCEVNVDDCPGHRC
LNGGTCVDGVNTYNCQCPPEWTGQFCTEDVDECQL
QPNACHNGGTCFNTLGGHSCVCVNGWTGESCSQNI
DDCATAVCFHGATCHDRVASFYCACPMGKTGLLCH
LDDACVSNPCHEDAICDTNPVNGRAICTCPPGFTG
GACDQDVDECSIGANPCEHLGRCVNTQGSFLCQCG
RGYTGPRCETDVNECLSGPCRNQATCLDRIGQFTC
ICMAGFTGTYCEVDIDECQSSPCVNGGVCKDRVNG
FSCTCPSGFSGSTCQLDVDECASTPCRNGAKCVDQ
PDGYECRCAEGFEGTLCDRNVDDCSPDPCHHGRCV
DGIASFSCACAPGYTGTRCESQVDECRSQPCRHGG
KCLDLVDKYLCRCPSGTTGVNCEVNIDDCASNPCT
FGVCRDGINRYDCVCQPGFTGPLCNVEINECASSP
CGEGGSCVDGENGFRCLCPPGSLPPLCLPPSHPCA
HEPCSHGICYDAPGGFRCVCEPGWSGPRCSQSLAR
DACESQPCRAGGTCSSDGMGFHCTCPPGVQGRQCE
LLSPCTPNPCEHGGRCESAPGQLPVCSCPQGWQGP
RCQQDVDECAGPAPCGPHGICTNLAGSFSCTCHGG
YTGPSCDQDINDCDPNPCLNGGSCQDGVGSFSCSC
LPGFAGPRCARDVDECLSNPCGPGTCTDHVASFTC
TCPPGYGGFHCEQDLPDCSPSSCFNGGTCVDGVNS
FSCLCRPGYTGAHCQHEADPCLSRPCLHGGVCSAA
HPGFRCTCLESFTGPQCQTLVDWCSRQPCQNGGRC
VQTGAYCLCPPGWSGRLCDIRSLPCREAAAQIGVR
LEQLCQAGGQCVDEDSSHYCVCPEGRTGSHCEQEV
DPCLAQPCQHGGTCRGYMGGYMCECLPGYNGDNCE
DDVDECASQPCQHGGSCIDLVARYLCSCPPGTLGV
LCEINEDDCGPGPPLDSGPRCLHNGTCVDLVGGFR
CTCPPGYTGLRCEADINECRSGACHAAHTRDCLQD
PGGGFRCLCHAGFSGPRCQTVLSPCESQPCQHGGQ
CRPSPGPGGGLTFTCHCAQPFWGPRCERVARSCRE
LQCPVGVPCQQTPRGPRCACPPGLSGPSCRSFPGS
PPGASNASCAAAPCLHGGSCRPAPLAPFFRCACAQ
GWTGPRCEAPAAAPEVSEEPRCPRAACQAKRGDQR
CDRECNSPGCGWDGGDCSLSVGDPWRQCEALQCWR
LFNNSRCDPACSSPACLYDNFDCHAGGRERTCNPV
YEKYCADHFADGRCDQGCNTEECGWDGLDCASEVP
ALLARGVLVLTVLLPPEELLRSSADFLQRLSAILR
TSLRFRLDAHGQAMVFPYHRPSPGSEPRARRELAP
EVIGSVVMLEIDNRLCLQSPENDHCFPDAQSAADY
LGALSAVERLDFPYPLRDVRGEPLEPPEPSVPLLP
LLVAGAVLLLVILVLGVMVARRKREHSTLWFPEGF
SLHKDVASGHKGRREPVGQDALGMKNMAKGESLMG
EVATDWMDTECPEAKRLKVEEPGMGAEEAVDCRQW
TQHHLVAADIRVAPAMALTPPQGDADADGMDVNVR
GPDGFTPLMLASFCGGALEPMPTEEDEADDTSASI
ISDLICQGAQLGARTDRTGETALHLAARYARADAA
KRLLDAGADTNAQDHSGRTPLHTAVTADAQGVFQI
LIRNRSTDLDARMADGSTALILAARLAVEGMVEEL
IASHADVNAVDELGKSALHWAAAVNNVEATLALLK
NGANKDMQDSKEETPLFLAAREGSYEAAKLLLDHF
ANREITDHLDRLPRDVAQERLHQDIVRLLDQPSGP
RSPPGPHGLGPLLCPPGAFLPGLKAAQSGSKKSRR
PPGKAGLGPQGPRGRGKKLTLACPGPLADSSVTLS
PVDSLDSPRPFGGPPASPGGFPLEGPYAAATATAV
SLAQLGGPGRAGLGRQPPGGCVLSLGLLNPVAVPL
DWARLPPPAPPGPSFLLPLAPGPQLLNPGTPVSPQ
ERPPPYLAVPGHGEEYPAAGAHSSPPKARFLRVPS
EHPYLTPSPESPEHWASPSPPSLSDWSESTPSPAT
ATGAMATTTGALPAQPLPLSVPSSLAQAQTQLGPQ
PEVTPKRQVLA

A nucleotide sequence that encodes human NOTCH3 is publically available in the GenBank database under accession number NM_000435.2 and is as follows (the start and stop codons are underlined and bolded):

(SEQ ID No. 19)
GCGGCGCGGAGGCTGGCCCGGGACGCGCCCGGAGC
CCAGGGAAGGAGGGAGGAGGGGAGGGTCGCGGCCG
GCCGCCATGGGGCCGGGGGCCCGTGGCCGCCGCCG
CCGCCGTCGCCCGATGTCGCCGCCACCGCCACCGC
CACCCGTGCGGGCGCTGCCCCTGCTGCTGCTGCTA
GCGGGGCCGGGGGCTGCAGCCCCCCCTTGCCTGGA
CGGAAGCCCGTGTGCAAATGGAGGTCGTTGCACCC
AGCTGCCCTCCCGGGAGGCTGCCTGCCTGTGCCCG
CCTGGCTGGGTGGGTGAGCGGTGTCAGCTGGAGGA
CCCCTGTCACTCAGGCCCCTGTGCTGGCCGTGGTG
TCTGCCAGAGTTCAGTGGTGGCTGGCACCGCCCGA
TTCTCATGCCGGTGCCCCCGTGGCTTCCGAGGCCC
TGACTGCTCCCTGCCAGATCCCTGCCTCAGCAGCC
CTTGTGCCCACGGTGCCCGCTGCTCAGTGGGGCCC
GATGGACGCTTCCTCTGCTCCTGCCCACCTGGCTA
CCAGGGCCGCAGCTGCCGAAGCGACGTGGATGAGT
GCCGGGTGGGTGAGCCCTGCCGCCATGGTGGCACC
TGCCTCAACACACCTGGCTCCTTCCGCTGCCAGTG
TCCAGCTGGCTACACAGGGCCACTATGTGAGAACC
CCGCGGTGCCCTGTGCACCCTCACCATGCCGTAAC
GGGGGCACCTGCAGGCAGAGTGGCGACCTCACTTA
CGACTGTGCCTGTCTTCCTGGGTTTGAGGGTCAGA
ATTGTGAAGTGAACGTGGACGACTGTCCAGGACAC
CGATGTCTCAATGGGGGGACATGCGTGGATGGCGT
CAACACCTATAACTGCCAGTGCCCTCCTGAGTGGA
CAGGCCAGTTCTGCACGGAGGACGTGGATGAGTGT
CAGCTGCAGCCCAACGCCTGCCACAATGGGGGTAC
CTGCTTCAACACGCTGGGTGGCCACAGCTGCGTGT
GTGTCAATGGCTGGACAGGCGAGAGCTGCAGTCAG
AATATCGATGACTGTGCCACAGCCGTGTGCTTCCA
TGGGGCCACCTGCCATGACCGCGTGGCTTCTTTCT
ACTGTGCCTGCCCCATGGGCAAGACTGGCCTCCTG
TGTCACCTGGATGACGCCTGTGTCAGCAACCCCTG
CCACGAGGATGCTATCTGTGACACAAATCCGGTGA
ACGGCCGGGCCATTTGCACCTGTCCTCCCGGCTTC
ACGGGTGGGGCATGTGACCAGGATGTGGACGAGTG
CTCTATCGGCGCCAACCCCTGCGAGCACTTGGGCA
GGTGCGTGAACACGCAGGGCTCCTTCCTGTGCCAG
TGCGGTCGTGGCTACACTGGACCTCGCTGTGAGAC
CGATGTCAACGAGTGTCTGTCGGGGCCCTGCCGAA
ACCAGGCCACGTGCCTCGACCGCATAGGCCAGTTC
ACCTGTATCTGTATGGCAGGCTTCACAGGAACCTA
TTGCGAGGTGGACATTGACGAGTGTCAGAGTAGCC
CCTGTGTCAACGGTGGGGTCTGCAAGGACCGAGTC
AATGGCTTCAGCTGCACCTGCCCCTCGGGCTTCAG
CGGCTCCACGTGTCAGCTGGACGTGGACGAATGCG
CCAGCACGCCCTGCAGGAATGGCGCCAAATGCGTG
GACCAGCCCGATGGCTACGAGTGCCGCTGTGCCGA
GGGCTTTGAGGGCACGCTGTGTGATCGCAACGTGG
ACGACTGCTCCCCTGACCCATGCCACCATGGTCGC
TGCGTGGATGGCATCGCCAGCTTCTCATGTGCCTG
TGCTCCTGGCTACACGGGCACACGCTGCGAGAGCC
AGGTGGACGAATGCCGCAGCCAGCCCTGCCGCCAT
GGCGGCAAATGCCTAGACCTGGTGGACAAGTACCT
CTGCCGCTGCCCTTCTGGGACCACAGGTGTGAACT
GCGAAGTGAACATTGACGACTGTGCCAGCAACCCC
TGCACCTTTGGAGTCTGCCGTGATGGCATCAACCG
CTACGACTGTGTCTGCCAACCTGGCTTCACAGGGC
CCCTTTGTAACGTGGAGATCAATGAGTGTGCTTCC
AGCCCATGCGGCGAGGGAGGTTCCTGTGTGGATGG
GGAAAATGGCTTCCGCTGCCTCTGCCCGCCTGGCT
CCTTGCCCCCACTCTGCCTCCCCCCGAGCCATCCC
TGTGCCCATGAGCCCTGCAGTCACGGCATCTGCTA
TGATGCACCTGGCGGGTTCCGCTGTGTGTGTGAGC
CTGGCTGGAGTGGCCCCCGCTGCAGCCAGAGCCTG
GCCCGAGACGCCTGTGAGTCCCAGCCGTGCAGGGC
CGGTGGGACATGCAGCAGCGATGGAATGGGTTTCC
ACTGCACCTGCCCGCCTGGTGTCCAGGGACGTCAG
TGTGAACTCCTCTCCCCCTGCACCCCGAACCCCTG
TGAGCATGGGGGCCGCTGCGAGTCTGCCCCTGGCC
AGCTGCCTGTCTGCTCCTGCCCCCAGGGCTGGCAA
GGCCCACGATGCCAGCAGGATGTGGACGAGTGTGC
TGGCCCCGCACCCTGTGGCCCTCATGGTATCTGCA
CCAACCTGGCAGGGAGTTTCAGCTGCACCTGCCAT
GGAGGGTACACTGGCCCTTCCTGCGATCAGGACAT
CAATGACTGTGACCCCAACCCATGCCTGAACGGTG
GCTCGTGCCAAGACGGCGTGGGCTCCTTTTCCTGC
TCCTGCCTCCCTGGTTTCGCCGGCCCACGATGCGC
CCGCGATGTGGATGAGTGCCTGAGCAACCCCTGCG
GCCCGGGCACCTGTACCGACCACGTGGCCTCCTTC
ACCTGCACCTGCCCGCCAGGCTACGGAGGCTTCCA
CTGCGAACAGGACCTGCCCGACTGCAGCCCCAGCT
CCTGCTTCAATGGCGGGACCTGTGTGGACGGCGTG
AACTCGTTCAGCTGCCTGTGCCGTCCCGGCTACAC
AGGAGCCCACTGCCAACATGAGGCAGACCCCTGCC
TCTCGCGGCCCTGCCTACACGGGGGCGTCTGCAGC
GCCGCCCACCCTGGCTTCCGCTGCACCTGCCTCGA
GAGCTTCACGGGCCCGCAGTGCCAGACGCTGGTGG
ATTGGTGCAGCCGCCAGCCTTGTCAAAACGGGGGT
CGCTGCGTCCAGACTGGGGCCTATTGCCTTTGTCC
CCCTGGATGGAGCGGACGCCTCTGTGACATCCGAA
GCTTGCCCTGCAGGGAGGCCGCAGCCCAGATCGGG
GTGCGGCTGGAGCAGCTGTGTCAGGCGGGTGGGCA
GTGTGTGGATGAAGACAGCTCCCACTACTGCGTGT
GCCCAGAGGGCCGTACTGGTAGCCACTGTGAGCAG
GAGGTGGACCCCTGCTTGGCCCAGCCCTGCCAGCA
TGGGGGGACCTGCCGTGGCTATATGGGGGGCTACA
TGTGTGAGTGTCTTCCTGGCTACAATGGTGATAAC
TGTGAGGACGACGTGGACGAGTGTGCCTCCCAGCC
CTGCCAGCACGGGGGTTCATGCATTGACCTCGTGG
CCCGCTATCTCTGCTCCTGTCCCCCAGGAACGCTG
GGGGTGCTCTGCGAGATTAATGAGGATGACTGCGG
CCCAGGCCCACCGCTGGACTCAGGGCCCCGGTGCC
TACACAATGGCACCTGCGTGGACCTGGTGGGTGGT
TTCCGCTGCACCTGTCCCCCAGGATACACTGGTTT
GCGCTGCGAGGCAGACATCAATGAGTGTCGCTCAG
GTGCCTGCCACGCGGCACACACCCGGGACTGCCTG
CAGGACCCAGGCGGAGGTTTCCGTTGCCTTTGTCA
TGCTGGCTTCTCAGGTCCTCGCTGTCAGACTGTCC
TGTCTCCCTGCGAGTCCCAGCCATGCCAGCATGGA
GGCCAGTGCCGTCCTAGCCCGGGTCCTGGGGGTGG
GCTGACCTTCACCTGTCACTGTGCCCAGCCGTTCT
GGGGTCCGCGTTGCGAGCGGGTGGCGCGCTCCTGC
CGGGAGCTGCAGTGCCCGGTGGGCGTCCCATGCCA
GCAGACGCCCCGCGGGCCGCGCTGCGCCTGCCCCC
CAGGGTTGTCGGGACCCTCCTGCCGCAGCTTCCCG
GGGTCGCCGCCGGGGGCCAGCAACGCCAGCTGCGC
GGCCGCCCCCTGTCTCCACGGGGGCTCCTGCCGCC
CCGCGCCGCTCGCGCCCTTCTTCCGCTGCGCTTGC
GCGCAGGGCTGGACCGGGCCGCGCTGCGAGGCGCC
CGCCGCGGCACCCGAGGTCTCGGAGGAGCCGCGGT
GCCCGCGCGCCGCCTGCCAGGCCAAGCGCGGGGAC
CAGCGCTGCGACCGCGAGTGCAACAGCCCAGGCTG
CGGCTGGGACGGCGGCGACTGCTCGCTGAGCGTGG
GCGACCCCTGGCGGCAATGCGAGGCGCTGCAGTGC
TGGCGCCTCTTCAACAACAGCCGCTGCGACCCCGC
CTGCAGCTCGCCCGCCTGCCTCTACGACAACTTCG
ACTGCCACGCCGGTGGCCGCGAGCGCACTTGCAAC
CCGGTGTACGAGAAGTACTGCGCCGACCACTTTGC
CGACGGCCGCTGCGACCAGGGCTGCAACACGGAGG
AGTGCGGCTGGGATGGGCTGGATTGTGCCAGCGAG
GTGCCGGCCCTGCTGGCCCGCGGCGTGCTGGTGCT
CACAGTGCTGCTGCCGCCAGAGGAGCTACTGCGTT
CCAGCGCCGACTTTCTGCAGCGGCTCAGCGCCATC
CTGCGCACCTCGCTGCGCTTCCGCCTGGACGCGCA
CGGCCAGGCCATGGTCTTCCCTTACCACCGGCCTA
GTCCTGGCTCCGAACCCCGGGCCCGTCGGGAGCTG
GCCCCCGAGGTGATCGGCTCGGTAGTAATGCTGGA
GATTGACAACCGGCTCTGCCTGCAGTCGCCTGAGA
ATGATCACTGCTTCCCCGATGCCCAGAGCGCCGCT
GACTACCTGGGAGCGTTGTCAGCGGTGGAGCGCCT
GGACTTCCCGTACCCACTGCGGGACGTGCGGGGGG
AGCCGCTGGAGCCTCCAGAACCCAGCGTCCCGCTG
CTGCCACTGCTAGTGGCGGGCGCTGTCTTGCTGCT
GGTCATTCTCGTCCTGGGTGTCATGGTGGCCCGGC
GCAAGCGCGAGCACAGCACCCTCTGGTTCCCTGAG
GGCTTCTCACTGCACAAGGACGTGGCCTCTGGTCA
CAAGGGCCGGCGGGAACCCGTGGGCCAGGACGCGC
TGGGCATGAAGAACATGGCCAAGGGTGAGAGCCTG
ATGGGGGAGGTGGCCACAGACTGGATGGACACAGA
GTGCCCAGAGGCCAAGCGGCTAAAGGTAGAGGAGC
CAGGCATGGGGGCTGAGGAGGCTGTGGATTGCCGT
CAGTGGACTCAACACCATCTGGTTGCTGCTGACAT
CCGCGTGGCACCAGCCATGGCACTGACACCACCAC
AGGGCGACGCAGATGCTGATGGCATGGATGTCAAT
GTGCGTGGCCCAGATGGCTTCACCCCGCTAATGCT
GGCTTCCTTCTGTGGGGGGGCTCTGGAGCCAATGC
CAACTGAAGAGGATGAGGCAGATGACACATCAGCT
AGCATCATCTCCGACCTGATCTGCCAGGGGGCTCA
GCTTGGGGCACGGACTGACCGTACTGGCGAGACTG
CTTTGCACCTGGCTGCCCGTTATGCCCGTGCTGAT
GCAGCCAAGCGGCTGCTGGATGCTGGGGCAGACAC
CAATGCCCAGGACCACTCAGGCCGCACTCCCCTGC
ACACAGCTGTCACAGCCGATGCCCAGGGTGTCTTC
CAGATTCTCATCCGAAACCGCTCTACAGACTTGGA
TGCCCGCATGGCAGATGGCTCAACGGCACTGATCC
TGGCGGCCCGCCTGGCAGTAGAGGGCATGGTGGAA
GAGCTCATCGCCAGCCATGCTGATGTCAATGCTGT
GGATGAGCTTGGGAAATCAGCCTTACACTGGGCTG
CGGCTGTGAACAACGTGGAAGCCACTTTGGCCCTG
CTCAAAAATGGAGCCAATAAGGACATGCAGGATAG
CAAGGAGGAGACCCCCCTATTCCTGGCCGCCCGCG
AGGGCAGCTATGAGGCTGCCAAGCTGCTGTTGGAC
CACTTTGCCAACCGTGAGATCACCGACCACCTGGA
CAGGCTGCCGCGGGACGTAGCCCAGGAGAGACTGC
ACCAGGACATCGTGCGCTTGCTGGATCAACCCAGT
GGGCCCCGCAGCCCCCCCGGTCCCCACGGCCTGGG
GCCTCTGCTCTGTCCTCCAGGGGCCTTCCTCCCTG
GCCTCAAAGCGGCACAGTCGGGGTCCAAGAAGAGC
AGGAGGCCCCCCGGGAAGGCGGGGCTGGGGCCGCA
GGGGCCCCGGGGGCGGGGCAAGAAGCTGACGCTGG
CCTGCCCGGGCCCCCTGGCTGACAGCTCGGTCACG
CTGTCGCCCGTGGACTCGCTGGACTCCCCGCGGCC
TTTCGGTGGGCCCCCTGCTTCCCCTGGTGGCTTCC
CCCTTGAGGGGCCCTATGCAGCTGCCACTGCCACT
GCAGTGTCTCTGGCACAGCTTGGTGGCCCAGGCCG
GGCGGGTCTAGGGCGCCAGCCCCCTGGAGGATGTG
TACTCAGCCTGGGCCTGCTGAACCCTGTGGCTGTG
CCCCTCGATTGGGCCCGGCTGCCCCCACCTGCCCC
TCCAGGCCCCTCGTTCCTGCTGCCACTGGCGCCGG
GACCCCAGCTGCTCAACCCAGGGACCCCCGTCTCC
CCGCAGGAGCGGCCCCCGCCTTACCTGGCAGTCCC
AGGACATGGCGAGGAGTACCCGGCGGCTGGGGCAC
ACAGCAGCCCCCCAAAGGCCCGCTTCCTGCGGGTT
CCCAGTGAGCACCCTTACCTGACCCCATCCCCCGA
ATCCCCTGAGCACTGGGCCAGCCCCTCACCTCCCT
CCCTCTCAGACTGGTCCGAATCCACGCCTAGCCCA
GCCACTGCCACTGGGGCCATGGCCACCACCACTGG
GGCACTGCCTGCCCAGCCACTTCCCTTGTCTGTTC
CCAGCTCCCTTGCTCAGGCCCAGACCCAGCTGGGG
CCCCAGCCGGAAGTTACCCCCAAGAGGCAAGTGTT
GGCCTGAGACGCTCGTCAGTTCTTAGATCTTGGGG
GCCTAAAGAGACCCCCGTCCTGCCTCCTTTCTTTC
TCTGTCTCTTCCTTCCTTTTAGTCTTTTTCATCCT
CTTCTCTTTCCACCAACCCTCCTGCATCCTTGCCT
TGCAGCGTGACCGAGATAGGTCATCAGCCCAGGGC
TTCAGTCTTCCTTTATTTATAATGGGTGGGGGCTA
CCACCCACCCTCTCAGTCTTGTGAAGAGTCTGGGA
CCTCCTTCTTCCCCACTTCTCTCTTCCCTCATTCC
TTTCTCTCTCCTTCTGGCCTCTCATTTCCTTACAC
TCTGACATGAATGAATTATTATTATTTTTATTTTT
CTTTTTTTTTTTACATTTTGTATAGAAACAAATTC
ATTTAAACAAACTTATTATTATTATTTTTTACAAA
ATATATATATGGAGATGCTCCCTCCCCCTGTGAAC
CCCCCAGTGCCCCCGTGGGGCTGAGTCTGTGGGCC
CATTCGGCCAAGCTGGATTCTGTGTACCTAGTACA
CAGGCATGACTGGGATCCCGTGTACCGAGTACACG
ACCCAGGTATGTACCAAGTAGGCACCCTTGGGCGC
ACCCACTGGGGCCAGGGGTCGGGGGAGTGTTGGGA
GCCTCCTCCCCACCCCACCTCCCTCACTTCACTGC
ATTCCAGATGGGACATGTTCCATAGCCTTGCTGGG
GAAGGGCCCACTGCCAACTCCCTCTGCCCCAGCCC
CACCCTTGGCCATCTCCCTTTGGGAACTAGGGGGC
TGCTGGTGGGAAATGGGAGCCAGGGCAGATGTATG
CATTCCTTTGTGTCCCTGTAAATGTGGGACTACAA
GAAGAGGAGCTGCCTGAGTGGTACTTTCTCTTCCT
GGTAATCCTCTGGCCCAGCCTCATGGCAGAATAGA
GGTATTTTTAGGCTATTTTTGTAATATGGCTTCTG
GTCAAAATCCCTGTGTAGCTGAATTCCCAAGCCCT
GCATTGTACAGCCCCCCACTCCCCTCACCACCTAA
TAAAGGAATAGTTAACACTCAAAAAAAAAAAAAAA
AAAA

In a NOTCH3-encoding mRNA sequence, each “T” in the sequence above would be a “U”.

Another amino acid sequence for human NOTCH3 is publically available in the GenBank database under accession number AAB91371.1 and is as follows:

(SEQ ID No. 20)
MGPGARGRRRRRRPMSPPPPPPPVRALPLLLLLAG
PGAAAPPCLDGSPCANGGRCTQLPSREAACLCPPG
WVGERCQLEDPCHSGPCAGRGVCQSSVVAGTARFS
CRCPRGFRGPDCSLPDPCLSSPCAHGARCSVGPDG
RFLCSCPPGYQGRSCRSDVDECRVGEPCRHGGTCL
NTPGSFRCQCPAGYTGPLCENPAVPCAPSPCRNGG
TCRQSGDLTYDCACLPGFEGQNCEVNVDDCPGHRC
LNGGTCVDGVNTYNCQCPPEWTGQFCTEDVDECQL
QPNACHNGGTCFNTLGGHSCVCVNGWTGESCSQNI
DDCATAVCFHGATCHDRVASFYCACPMGKTGLLCH
LDDACVSNPCHEDAICDTNPVNGRAICTCPPGFTG
GACDQDVDECSIGANPCEHLGRCVNTQGSFLCQCG
RGYTGPRCETDVNECLSGPCRNQATCLDRIGQFTC
ICMAGFTGTYCEVDIDECQSSPCVNGGVCKDRVNG
FSCTCPSGFSGSTCQLDVDECASTPCRNGAKCVDQ
PDGYECRCAEGFEGTLCDRNVDDCSPDPCHHGRCV
DGIASFSCACAPGYTGTRCESQVDECRSQPCRHGG
KCLDLVDKYLCRCPSGTTGVNCEVNIDDCASNPCT
FGVCRDGINRYDCVCQPGFTGPLCNVEINECASSP
CGEGGSCVDGENGFRCLCPPGSLPPLCLPPSHPCA
HEPCSHGICYDAPGGFRCVCEPGWSGPRCSQSLAR
DACESQPCRAGGTCSSDGMGFHCTCPPGVQGRQCE
LLSPCTPNPCEHGGRCESAPGQLPVCSCPQGWQGP
RCQQDVDECAGPAPCGPHGICTNLAGSFSCTCHGG
YTGPSCDQDINDCDPNPCLNGGSCQDGVGSFSCSC
LPGFAGPRCARDVDECLSNPCGPGTCTDHVASFTC
TCPPGYGGFHCEQDLPDCSPSSCFNGGTCVDGVNS
FSCLCRPGYTGAHCQHEADPCLSRPCLHGGVCSAA
HPGFRCTCLESFTGPQCQTLVDWCSRQPCQNGGRC
VQTGAYCLCPPGWSGRLCDIRSLPCREAAAQIGVR
LEQLCQAGGQCVDEDSSHYCVCPEGRTGSHCEQEV
DPCLAQPCQHGGTCRGYMGGYMCECLPGYNGDNCE
DDVDECASQPCQHGGSCIDLVARYLCSCPPGTLGV
LCEINEDDCGPGPPLDSGPRCLHNGTCVDLVGGFR
CTCPPGYTGLRCEADINECRSGACHAAHTRDCLQD
PGGGFRCLCHAGFSGPRCQTVLSPCESQPCQHGGQ
CRPSPGPGGGLTFTCHCAQPFWGPRCERVARSCRE
LQCPVGVPCQQTPRGPRCACPPGLSGPSCRSFPGS
PPGASNASCAAAPCLHGGSCRPAPLAPFFRCACAQ
GWTGPRCEAPAAAPEVSEEPRCPRAACQAKRGDQR
CDRECNSPGCGWDGGDCSLSVGDPWRQCEALQCWR
LFNNSRCDPACSSPACLYDNFDCHAGGRERTCNPV
YEKYCADHFADGRCDQGCNTEECGWDGLDCASEVP
ALLARGVLVLTVLLPPEELLRSSADFLQRLSAILR
TSLRFRLDAHGQAMVFPYHRPSPGSEPRARRELAP
EVIGSVVMLEIDNRLCLQSPENDHCFPDAQSAADY
LGALSAVERLDFPYPLRDVRGEPLEPPEPSVPLLP
LLVAGAVLLLVILVLGVMVARRKREHSTLWFPEGF
SLHKDVASGHKGRREPVGQDALGMKNMAKGESLMG
EVATDWMDTECPEAKRLKVEEPGMGAEEAVDCRQW
TQHHLVAADIRVAPAMALTPPQGDADADGMDVNVR
GPDGFTPLMLASFCGGALEPMPTEEDEADDTSASI
ISDLICQGAQLGARTDRTGETALHLAARYARADAA
KRLLDAGADTNAQDHSGRTPLHTAVTADAQGVFQI
LIRNRSTDLDARMADGSTALILAARLAVEGMVEEL
IASHADVNAVDELGKSALHWAAAVNNVEATLALLK
NGANKDMQDSKEETPLFLAAREGSYEAAKLLLDHF
ANREITDHLDRLPRDVAQERLHQDIVRLLDQPSGP
RSPPGPHGLGPLLCPPGAFLPGLKAAQSGSKKSRR
PPGKAGLGPQGPRGRGKKLTLACPGPLADSSVTLS
PVDSLDSPRPFGGPPASPGGFPLEGPYAAATATAV
SLAQLGGPGRAGLGRQPPGGCVLSLGLLNPVAVPL
DWARLPPPAPPGPSFLLPLAPGPQLLNPGTPVSPQ
ERPPPYLAVPGHGEEYPVAGAHSSPPKARFLRVPS
EHPYLTPSPESPEHWASPSPPSLSDWSESTPSPAT
ATGAMATTTGALPAQPLPLSVPSSLAQAQTQLGPQ
PEVTPKRQVLA

A nucleotide sequence that encodes human NOTCH3 is publically available in the GenBank database under accession number U97669.1 (SEQ ID NO: 2) and is as follows (the start and stop codons are underlined and bolded):

ACGCGGCGCGGAGGCTGGCCCGGGACGCGCCCGGA
GCCCAGGGAAGGAGGGAGGAGGGGAGGGTCGCGGC
CGGCCGCCATGGGGCCGGGGGCCCGTGGCCGCCGC
CGCCGCCGTCGCCCGATGTCGCCGCCACCGCCACC
GCCACCCGTGCGGGCGCTGCCCCTGCTGCTGCTGC
TAGCGGGGCCGGGGGCTGCAGCCCCCCCTTGCCTG
GACGGAAGCCCGTGTGCAAATGGAGGTCGTTGCAC
CCAGCTGCCCTCCCGGGAGGCTGCCTGCCTGTGCC
CGCCTGGCTGGGTGGGTGAGCGGTGTCAGCTGGAG
GACCCCTGTCACTCAGGCCCCTGTGCTGGCCGTGG
TGTCTGCCAGAGTTCAGTGGTGGCTGGCACCGCCC
GATTCTCATGCCGGTGCCCCCGTGGCTTCCGAGGC
CCTGACTGCTCCCTGCCAGATCCCTGCCTCAGCAG
CCCTTGTGCCCACGGTGCCCGCTGCTCAGTGGGGC
CCGATGGACGCTTCCTCTGCTCCTGCCCACCTGGC
TACCAGGGCCGCAGCTGCCGAAGCGACGTGGATGA
GTGCCGGGTGGGTGAGCCCTGCCGCCATGGTGGCA
CCTGCCTCAACACACCTGGCTCCTTCCGCTGCCAG
TGTCCAGCTGGCTACACAGGGCCACTATGTGAGAA
CCCCGCGGTGCCCTGTGCGCCCTCACCATGCCGTA
ACGGGGGCACCTGCAGGCAGAGTGGCGACCTCACT
TACGACTGTGCCTGTCTTCCTGGGTTTGAGGGTCA
GAATTGTGAAGTGAACGTGGACGACTGTCCAGGAC
ACCGATGTCTCAATGGGGGGACATGCGTGGATGGC
GTCAACACCTATAACTGCCAGTGCCCTCCTGAGTG
GACAGGCCAGTTCTGCACGGAGGACGTGGATGAGT
GTCAGCTGCAGCCCAACGCCTGCCACAATGGGGGT
ACCTGCTTCAACACGCTGGGTGGCCACAGCTGCGT
GTGTGTCAATGGCTGGACAGGTGAGAGCTGCAGTC
AGAATATCGATGACTGTGCCACAGCCGTGTGCTTC
CATGGGGCCACCTGCCATGACCGCGTGGCTTCTTT
CTACTGTGCCTGCCCCATGGGCAAGACTGGCCTCC
TGTGTCACCTGGATGACGCCTGTGTCAGCAACCCC
TGCCACGAGGATGCTATCTGTGACACAAATCCGGT
GAACGGCCGGGCCATTTGCACCTGTCCTCCCGGCT
TCACGGGTGGGGCATGTGACCAGGATGTGGACGAG
TGCTCTATCGGCGCCAACCCCTGCGAGCACTTGGG
CAGGTGCGTGAACACGCAGGGCTCCTTCCTGTGCC
AGTGCGGTCGTGGCTACACTGGACCTCGCTGTGAG
ACCGATGTCAACGAGTGTCTGTCGGGGCCCTGCCG
AAACCAGGCCACGTGCCTCGACCGCATAGGCCAGT
TCACCTGTATCTGTATGGCAGGCTTCACAGGAACC
TATTGCGAGGTGGACATTGACGAGTGTCAGAGTAG
CCCCTGTGTCAACGGTGGGGTCTGCAAGGACCGAG
TCAATGGCTTCAGCTGCACCTGCCCCTCGGGCTTC
AGCGGCTCCACGTGTCAGCTGGACGTGGACGAATG
CGCCAGCACGCCCTGCAGGAATGGCGCCAAATGCG
TGGACCAGCCCGATGGCTACGAGTGCCGCTGTGCC
GAGGGCTTTGAGGGCACGCTGTGTGATCGCAACGT
GGACGACTGCTCCCCTGACCCATGCCACCATGGTC
GCTGCGTGGATGGCATCGCCAGCTTCTCATGTGCC
TGTGCTCCTGGCTACACGGGCACACGCTGCGAGAG
CCAGGTGGACGAATGCCGCAGCCAGCCCTGCCGCC
ATGGCGGCAAATGCCTAGACCTGGTGGACAAGTAC
CTCTGCCGCTGCCCTTCTGGGACCACAGGTGTGAA
CTGCGAAGTGAACATTGACGACTGTGCCAGCAACC
CCTGCACCTTTGGAGTCTGCCGTGATGGCATCAAC
CGCTACGACTGTGTCTGCCAACCTGGCTTCACAGG
GCCCCTTTGTAACGTGGAGATCAATGAGTGTGCTT
CCAGCCCATGCGGCGAGGGAGGTTCCTGTGTGGAT
GGGGAAAATGGCTTCCGCTGCCTCTGCCCGCCTGG
CTCCTTGCCCCCACTCTGCCTCCCCCCGAGCCATC
CCTGTGCCCATGAGCCCTGCAGTCACGGCATCTGC
TATGATGCACCTGGCGGGTTCCGCTGTGTGTGTGA
GCCTGGCTGGAGTGGCCCCCGCTGCAGCCAGAGCC
TGGCCCGAGACGCCTGTGAGTCCCAGCCGTGCAGG
GCCGGTGGGACATGCAGCAGCGATGGAATGGGTTT
CCACTGCACCTGCCCGCCTGGTGTCCAGGGACGTC
AGTGTGAACTCCTCTCCCCCTGCACCCCGAACCCC
TGTGAGCATGGGGGCCGCTGCGAGTCTGCCCCTGG
CCAGCTGCCTGTCTGCTCCTGCCCCCAGGGCTGGC
AAGGCCCACGATGCCAGCAGGATGTGGACGAGTGT
GCTGGCCCCGCACCCTGTGGCCCTCATGGTATCTG
CACCAACCTGGCAGGGAGTTTCAGCTGCACCTGCC
ATGGAGGGTACACTGGCCCTTCCTGTGATCAGGAC
ATCAATGACTGTGACCCCAACCCATGCCTGAACGG
TGGCTCGTGCCAAGACGGCGTGGGCTCCTTTTCCT
GCTCCTGCCTCCCTGGTTTCGCCGGCCCACGATGC
GCCCGCGATGTGGATGAGTGCCTGAGCAACCCCTG
CGGCCCGGGCACCTGTACCGACCACGTGGCCTCCT
TCACCTGCACCTGCCCGCCGGGCTACGGAGGCTTC
CACTGCGAACAGGACCTGCCCGACTGCAGCCCCAG
CTCCTGCTTCAATGGCGGGACCTGTGTGGACGGCG
TGAACTCGTTCAGCTGCCTGTGCCGTCCCGGCTAC
ACAGGAGCCCACTGCCAACATGAGGCAGACCCCTG
CCTCTCGCGGCCCTGCCTACACGGGGGCGTCTGCA
GCGCCGCCCACCCTGGCTTCCGCTGCACCTGCCTC
GAGAGCTTCACGGGCCCGCAGTGCCAGACGCTGGT
GGATTGGTGCAGCCGCCAGCCTTGTCAAAACGGGG
GTCGCTGCGTCCAGACTGGGGCCTATTGCCTTTGT
CCCCCTGGATGGAGCGGACGCCTCTGTGACATCCG
AAGCTTGCCCTGCAGGGAGGCCGCAGCCCAGATCG
GGGTGCGGCTGGAGCAGCTGTGTCAGGCGGGTGGG
CAGTGTGTGGATGAAGACAGCTCCCACTACTGCGT
GTGCCCAGAGGGCCGTACTGGTAGCCACTGTGAGC
AGGAGGTGGACCCCTGCTTGGCCCAGCCCTGCCAG
CATGGGGGGACCTGCCGTGGCTATATGGGGGGCTA
CATGTGTGAGTGTCTTCCTGGCTACAATGGTGATA
ACTGTGAGGACGACGTGGACGAGTGTGCCTCCCAG
CCCTGCCAGCACGGGGGTTCATGCATTGACCTCGT
GGCCCGCTATCTCTGCTCCTGTCCCCCAGGAACGC
TGGGGGTGCTCTGCGAGATTAATGAGGATGACTGC
GGCCCAGGCCCACCGCTGGACTCAGGGCCCCGGTG
CCTACACAATGGCACCTGCGTGGACCTGGTGGGTG
GTTTCCGCTGCACCTGTCCCCCAGGATACACTGGT
TTGCGCTGCGAGGCAGACATCAATGAGTGTCGCTC
AGGTGCCTGCCACGCGGCACACACCCGGGACTGCC
TGCAGGACCCAGGCGGAGGTTTCCGTTGCCTTTGT
CATGCTGGCTTCTCAGGTCCTCGCTGTCAGACTGT
CCTGTCTCCCTGCGAGTCCCAGCCATGCCAGCATG
GAGGCCAGTGCCGTCCTAGCCCGGGTCCTGGGGGT
GGGCTGACCTTCACCTGTCACTGTGCCCAGCCGTT
CTGGGGTCCGCGTTGCGAGCGGGTGGCGCGCTCCT
GCCGGGAGCTGCAGTGCCCGGTGGGCGTCCCATGC
CAGCAGACGCCCCGCGGGCCGCGCTGCGCCTGCCC
CCCAGGGTTGTCGGGACCCTCCTGCCGCAGCTTCC
CGGGGTCGCCGCCGGGGGCCAGCAACGCCAGCTGC
GCGGCCGCCCCCTGTCTCCACGGGGGCTCCTGCCG
CCCCGCGCCGCTCGCGCCCTTCTTCCGCTGCGCTT
GCGCGCAGGGCTGGACCGGGCCGCGCTGCGAGGCG
CCCGCCGCGGCACCCGAGGTCTCGGAGGAGCCGCG
GTGCCCGCGCGCCGCCTGCCAGGCCAAGCGCGGGG
ACCAGCGCTGCGACCGCGAGTGCAACAGCCCAGGC
TGCGGCTGGGACGGCGGCGACTGCTCGCTGAGCGT
GGGCGACCCCTGGCGGCAATGCGAGGCGCTGCAGT
GCTGGCGCCTCTTCAACAACAGCCGCTGCGACCCC
GCCTGCAGCTCGCCCGCCTGCCTCTACGACAACTT
CGACTGCCACGCCGGTGGCCGCGAGCGCACTTGCA
ACCCGGTGTACGAGAAGTACTGCGCCGACCACTTT
GCCGACGGCCGCTGCGACCAGGGCTGCAACACGGA
GGAGTGCGGCTGGGATGGGCTGGATTGTGCCAGCG
AGGTGCCGGCCCTGCTGGCCCGCGGCGTGCTGGTG
CTCACAGTGCTGCTGCCGCCGGAGGAGCTACTGCG
TTCCAGCGCCGACTTTCTGCAGCGGCTCAGCGCCA
TCCTGCGCACCTCGCTGCGCTTCCGCCTGGACGCG
CACGGCCAGGCCATGGTCTTCCCTTACCACCGGCC
TAGTCCTGGCTCCGAACCCCGGGCCCGTCGGGAGC
TGGCCCCCGAGGTGATCGGCTCGGTAGTAATGCTG
GAGATTGACAACCGGCTCTGCCTGCAGTCGCCTGA
GAATGATCACTGCTTCCCCGATGCCCAGAGCGCCG
CTGACTACCTGGGAGCGTTGTCAGCGGTGGAGCGC
CTGGACTTCCCGTACCCACTGCGGGACGTGCGGGG
GGAGCCGCTGGAGCCTCCAGAACCCAGCGTCCCGC
TGCTGCCACTGCTAGTGGCGGGCGCTGTCTTGCTG
CTGGTCATTCTCGTCCTGGGTGTCATGGTGGCCCG
GCGCAAGCGCGAGCACAGCACCCTCTGGTTCCCTG
AGGGCTTCTCACTGCACAAGGACGTGGCCTCTGGT
CACAAGGGCCGGCGGGAACCCGTGGGCCAGGACGC
GCTGGGCATGAAGAACATGGCCAAGGGTGAGAGCC
TGATGGGGGAGGTGGCCACAGACTGGATGGACACA
GAGTGCCCAGAGGCCAAGCGGCTAAAGGTAGAGGA
GCCAGGCATGGGGGCTGAGGAGGCTGTGGATTGCC
GTCAGTGGACTCAACACCATCTGGTTGCTGCTGAC
ATCCGCGTGGCACCAGCCATGGCACTGACACCACC
ACAGGGCGACGCAGATGCTGATGGCATGGATGTCA
ATGTGCGTGGCCCAGATGGCTTCACCCCGCTAATG
CTGGCTTCCTTCTGTGGGGGGGCTCTGGAGCCAAT
GCCAACTGAAGAGGATGAGGCAGATGACACATCAG
CTAGCATCATCTCCGACCTGATCTGCCAGGGGGCT
CAGCTTGGGGCACGGACTGACCGTACTGGCGAGAC
TGCTTTGCACCTGGCTGCCCGTTATGCCCGTGCTG
ATGCAGCCAAGCGGCTGCTGGATGCTGGGGCAGAC
ACCAATGCCCAGGACCACTCAGGCCGCACTCCCCT
GCACACAGCTGTCACAGCCGATGCCCAGGGTGTCT
TCCAGATTCTCATCCGAAACCGCTCTACAGACTTG
GATGCCCGCATGGCAGATGGCTCAACGGCACTGAT
CCTGGCGGCCCGCCTGGCAGTAGAGGGCATGGTGG
AAGAGCTCATCGCCAGCCATGCTGATGTCAATGCT
GTGGATGAGCTTGGGAAATCAGCCTTACACTGGGC
TGCGGCTGTGAACAACGTGGAAGCCACTTTGGCCC
TGCTCAAAAATGGAGCCAATAAGGACATGCAGGAT
AGCAAGGAGGAGACCCCCCTATTCCTGGCCGCCCG
CGAGGGCAGCTATGAGGCTGCCAAGCTGCTGTTGG
ACCACTTTGCCAACCGTGAGATCACCGACCACCTG
GACAGGCTGCCGCGGGACGTAGCCCAGGAGAGACT
GCACCAGGACATCGTGCGCTTGCTGGATCAACCCA
GTGGGCCCCGCAGCCCCCCCGGTCCCCACGGCCTG
GGGCCTCTGCTCTGTCCTCCAGGGGCCTTCCTCCC
TGGCCTCAAAGCGGCACAGTCGGGGTCCAAGAAGA
GCAGGAGGCCCCCCGGGAAGGCGGGGCTGGGGCCG
CAGGGGCCCCGGGGGCGGGGCAAGAAGCTGACGCT
GGCCTGCCCGGGCCCCCTGGCTGACAGCTCGGTCA
CGCTGTCGCCCGTGGACTCGCTGGACTCCCCGCGG
CCTTTCGGTGGGCCCCCTGCTTCCCCTGGTGGCTT
CCCCCTTGAGGGGCCCTATGCAGCTGCCACTGCCA
CTGCAGTGTCTCTGGCACAGCTTGGTGGCCCAGGC
CGGGCAGGTCTAGGGCGCCAGCCCCCTGGAGGATG
TGTACTCAGCCTGGGCCTGCTGAACCCTGTGGCTG
TGCCCCTCGATTGGGCCCGGCTGCCCCCACCTGCC
CCTCCAGGCCCCTCGTTCCTGCTGCCACTGGCGCC
GGGACCCCAGCTGCTCAACCCAGGGACCCCCGTCT
CCCCGCAGGAGCGGCCCCCGCCTTACCTGGCAGTC
CCAGGACATGGCGAGGAGTACCCGGTGGCTGGGGC
ACACAGCAGCCCCCCAAAGGCCCGCTTCCTGCGGG
TTCCCAGTGAGCACCCTTACCTGACCCCATCCCCC
GAATCCCCTGAGCACTGGGCCAGCCCCTCACCTCC
CTCCCTCTCAGACTGGTCCGAATCCACGCCTAGCC
CAGCCACTGCCACTGGGGCCATGGCCACCACCACT
GGGGCACTGCCTGCCCAGCCACTTCCCTTGTCTGT
TCCCAGCTCCCTTGCTCAGGCCCAGACCCAGCTGG
GGCCCCAGCCGGAAGTTACCCCCAAGAGGCAAGTG
TTGGCCTGAGACGCTCGTCAGTTCTTAGATCTTGG
GGGCCTAAAGAGACCCCCGTCCTGCCTCCTTTCTT
TCTCTGTCTCTTCCTTCCTTTTAGTCTTTTTCATC
CTCTTCTCTTTCCACCAACCCTCCTGCATCCTTGC
CTTGCAGCGTGACCGAGATAGGTCATCAGCCCAGG
GCTTCAGTCTTCCTTTATTTATAATGGGTGGGGGC
TACCACCCACCCTCTCAGTCTTGTGAAGAGTCTGG
GACCTCCTTCTTCCCCACTTCTCTCTTCCCTCATT
CCTTTCTCTCTCCTTCTGGCCTCTCATTTCCTTAC
ACTCTGACATGAATGAATTATTATTATTTTTCTTT
TTCTTTTTTTTTTTACATTTTGTATAGAAACAAAT
TCATTTAAACAAACTTATTATTATTATTTTTTACA
AAATATATATATGGAGATGCTCCCTCCCCCTGTGA
ACCCCCCAGTGCCCCCGTGGGGCTGAGTCTGTGGG
CCCATTCGGCCAAGCTGGATTCTGTGTACCTAGTA
CACAGGCATGACTGGGATCCCGTGTACCGAGTACA
CGACCCAGGTATGTACCAAGTAGGCACCCTTGGGC
GCACCCACTGGGGCCAGGGGTCGGGGGAGTGTTGG
GAGCCTCCTCCCCACCCCACCTCCCTCACTTCACT
GCATTCCAGATTGGACATGTTCCATAGCCTTGCTG
GGGAAGGGCCCACTGCCAACTCCCTCTGCCCCAGC
CCCACCCTTGGCCATCTCCCTTTGGGAACTAGGGG
GCTGCTGGTGGGAAATGGGAGCCAGGGCAGATGTA
TGCATTCCTTTATGTCCCTGTAAATGTGGGACTAC
AAGAAGAGGAGCTGCCTGAGTGGTACTTTCTCTTC
CTGGTAATCCTCTGGCCCAGCCTTATGGCAGAATA
GAGGTATTTTTAGGCTATTTTTGTAATATGGCTTC
TGGTCAAAATCCCTGTGTAGCTGAATTCCCAAGCC
CTGCATTGTACAGCCCCCCACTCCCCTCACCACCT
AATAAAGGAATAGTTAACACTCAAAAAAAAAAAAA
AAAAAA

In a NOTCH3-encoding mRNA sequence, each “T” in the sequence above would be a “U”.

The human NOTCH3 ectodomain sequence comprises amino acid positions 40 to 1571 of accession number Q9UM47. With respect to embodiments relating to CADASIL, the ectodomain comprises the extracellular domain until the furin cleavage site. This excludes the signal peptide from positions 1 to 39 and also excludes the 1572 to 2321 amino acid region encompassing a small portion that is extracellular, the transmembrane domain, and the intracellular domain.

An amino acid sequence for human N3ECD is:

(SEQ ID NO: 3)
APPCLDGSPC ANGGRCTQLP SREAACLCPP GWVGERCQLE DPCHSGPCAG
RGVCQSSVVAGTARFSCRCP RGFRGPDCSL PDPCLSSPCA HGARCSVGPD
GRFLCSCPPG YQGRSCRSDVDECRVGEPCR HGGTCLNTPG SFRCQCPAGY
TGPLCENPAV PCAPSPCRNG GTCRQSGDLTYDCACLPGFE GQNCEVNVDD
CPGHRCLNGG TCVDGVNTYN CQCPPEWTGQ FCTEDVDECQLQPNACHNGG
TCFNTLGGHS CVCVNGWTGE SCSQNIDDCA TAVCFHGATC HDRVASFYCA
CPMGKTGLLC HLDDACVSNP CHEDAICDTN PVNGRAICTC PPGFTGGACD
QDVDECSIGANPCEHLGRCV NTQGSFLCQC GRGYTGPRCE TDVNECLSGP
CRNQATCLDRIGQFTCICMAGFTGTYCEVD IDECQSSPCV NGGVCKDRVN
GFSCTCPSGF SGSTCQLDVD ECASTPCRNGAKCVDQPDGY ECRCAEGFEG
TLCDRNVDDC SPDPCHHGRC VDGIASFSCA CAPGYTGTRCESQVDECRSQ
PCRHGGKCLD LVDKYLCRCP SGTTGVNCEV NIDDCASNPC TFGVCRDGIN
RYDCVCQPGF TGPLCNVEIN ECASSPCGEG GSCVDGENGF RCLCPPGSLP
PLCLPPSHPCAHEPCSHGIC YDAPGGFRCV CEPGWSGPRC SQSLARDACE
SQPCRAGGTC SSDGMGFHCTCPPGVQGRQC ELLSPCTPNP CEHGGRCESA
PGQLPVCSCP QGWQGPRCQQ DVDECAGPAPCGPHGICTNL AGSFSCTCHG
GYTGPSCDQD INDCDPNPCL NGGSCQDGVG SFSCSCLPGFAGPRCARDVD
ECLSNPCGPG TCTDHVASFT CTCPPGYGGF HCEQDLPDCS PSSCFNGGTC
VDGVNSFSCL CRPGYTGAHC QHEADPCLSR PCLHGGVCSA AHPGFRCTCL
ESFTGPQCQTLVDWCSRQPC QNGGRCVQTG AYCLCPPGWS GRLCDIRSLP
CREAAAQIGV RLEQLCQAGGQCVDEDSSHY CVCPEGRTGS HCEQEVDPCL
AQPCQHGGTC RGYMGGYMCE CLPGYNGDNCEDDVDECASQ PCQHGGSCID
LVARYLCSCP PGTLGVLCEINEDDCGPGPP LDSGPRCLHNGTCVDLVGGF
RCTCPPGYTG LRCEADINEC RSGACHAAHT RDCLQDPGGG FRCLCHAGFS
GPRCQTVLSP CESQPCQHGG QCRPSPGPGG GLTFTCHCAQ PFWGPRCERV
ARSCRELQCPVGVPCQQTPR GPRCACPPGL SGPSCRSFPG SPPGASNASC
AAAPCLHGGS CRPAPLAPFFRCACAQGWTG PRCEAPAAAP EVSEEPRCPR
AACQAKRGDQ RCDRECNSPG CGWDGGDCSLSVGDPWRQCE ALQCWRLFNN
SRCDPACSSP ACLYDNFDCH AGGRERTCNP VYEKYCADHFADGRCDQGCN
TEECGWDGLD CASEVPALLA RGVLVLTVLL PPEELLRSSA DFLQRLSAILR
TSLRFRLDAHGQAMVFPYHR PSPGSEPRARR

The amino acid sequence for mouse N3ECD runs from positions 40 to 1572 of the amino acid sequence that is available in the UniProt database under accession number Q61982 (SEQ ID NO: 4), and is as follows:

(SEQ ID NO: 4)
APPCLDGSPC ANGGRCTHQQ PSLEAACLCL PGWVGERCQL EDPCHSGPCA
GRGVCQSSVVAGTARFSCRC LRGFQGPDCS QPDPCVSRPC VHGAPCSVGP
DGRFACACPP GYQGQSCQSDIDECRSGTTC RHGGTCLNTP GSFRCQCPLG
YTGLLCENPV VPCAPSPCRN GGTCRQSSDVTYDCACLPGF EGQNCEVNVD
DCPGHRCLNG GTCVDGVNTY NCQCPPEWTG QFCTEDVDECQLQPNACHNG
GTCFNLLGGH SCVCVNGWTGESCSQNIDDC ATAVCFHGAT CHDRVASFYC
ACPMGKTGLL CHLDDACVSN PCHEDAICDT NPVSGRAICT CPPGFTGGAC
DQDVDECSIGANPCEHLGRC VNTQGSFLCQ CGRGYTGPRC ETDVNECLSG
PCRNQATCLD RIGQFTCICMAGFTGTYCEV DIDECQSSPC VNGGVCKDRV
NGFSCTCPSG FSGSMCQLDV DECASTPCRNGAKCVDQPDG YECRCAEGFE
GTLCERNVDD CSPDPCHHGR CVDGIASFSC ACAPGYTGIRCESQVDECRS
QPCRYGGKCL DLVDKYLCRC PPGTTGVNCE VNIDDCASNP CTFGVCRDGI
NRYDCVCQPG FTGPLCNVEINECASSPCGE GGSCVDGENG FHCLCPPGSL
PPLCLPANHPCAHKPCSHGV CHDAPGGFRC VCEPGWSGPR CSQSLAPDAC
ESQPCQAGGT CTSDGIGFRCTCAPGFQGHQ CEVLSPCTPS LCEHGGHCES
DPDRLTVCSC PPGWQGPRCQ QDVDECAGASPCGPHGTCTN LPGNFRCICH
RGYTGPFCDQ DIDDCDPNPC LHGGSCQDGV GSFSCSCLDGFAGPRCARDV
DECLSSPCGP GTCTDHVASF TCACPPGYGG FHCEIDLPDC SPSSCFNGGT
CVDGVSSFSC LCRPGYTGTH CQYEADPCFS RPCLHGGICN PTHPGFECTC
REGFTGSQCQNPVDWCSQAP CQNGGRCVQT GAYCICPPGW SGRLCDIQSL
PCTEAAAQMG VRLEQLCQEGGKCIDKGRSH YCVCPEGRTG SHCEHEVDPC
TAQPCQHGGT CRGYMGGYVC ECPAGYAGDSCEDNIDECAS QPCQNGGSCI
DLVARYLCSC PPGTLGVLCEINEDDCDLGP SLDSGVQCLHNGTCVDLVGG
FRCNCPPGYT GLHCEADINE CRPGACHAAH TRDCLQDPGG HFRCVCHPGF
TGPRCQIALS PCESQPCQHG GQCRHSLGRG GGLTFTCHCV PPFWGLRCER
VARSCRELQCPVGIPCQQTA RGPRCACPPG LSGPSCRVSR ASPSGATNAS
CASAPCLHGG SCLPVQSVPFFRCVCAPGWG GPRCETPSAA PEVPEEPRCP
RAACQAKRGD QNCDRECNTP GCGWDGGDCSLNVDDPWRQC EALQCWRLFN
NSRCDPACSS PACLYDNFDC YSGGRDRTCN PVYEKYCADHFADGRCDQGC
NTEECGWDGL DCASEVPALL ARGVLVLTVL LPPEELLRSS ADFLQRLSAIL
RTSLRFRLDARGQAMVFPYH RPSPGSESRV RR

### Endostatin

Endostatin is a naturally-occurring, 20-kDa C-terminal fragment derived collagen18α1 (which is encoded by the COL18A1 gene). Endostatin is cleaved off collagen18α1. It is reported to serve as an anti-angiogenic agent, similar to angiostatin and thrombospondin. Endostatin is a broad-spectrum angiogenesis inhibitor and may interfere with the pro-angiogenic action of growth factors such as basic fibroblast growth factor (bFGF/FGF-2) and vascular endothelial growth factor (VEGF).

A binding agent (e.g., an antibody) that specifically binds endostatin may also bind full-length collagen18α1. In various embodiments, it is not necessary to distinguish endostatin that is detected from collagen18α1 (i.e., it is not necessary to rule out or determine that a portion of the endostatin detected is full-length collagen18α1).

An amino acid sequence for human endostatin is publically available in the UniProt database as positions 1572-1754 of accession number P39060 (SEQ ID NO: 5) and is as follows:

HSHRDFQPVLHLVALNSPLSGGMRGIRGADFQCFQQARAVGLAG
TFRAFLSSRLQDLYSIVRRADRAAVPIVNLKDELLFPSWEALFS
GSEGPLKPGARIFSFDGKDVLRHPTWPQKSVWHGSDPNGRRLTE
SYCETWRTEAPSATGQASSLLGGRLLGQSAASCHHAYIVLCIEN
SFMTASK

A nucleotide sequence that encodes human endostatin is publically available in the GenBank database as positions 4021-4569 of accession number NM_030582.3 (SEQ ID NO: 6) and is as follows:

CACAGCCACC GCGACTTCCA GCCGGTGCTC CACCTGGTTG
CGCTCAACAG CCCCCTGTCAGGCGGCATGC GGGGCATCCG
CGGGGCCGAC TTCCAGTGCT TCCAGCAGGC GCGGGCCGT
GGGGCTGGCGG GCACCTTCCG CGCCTTCCTG TCCTCGCGCC
TGCAGGACCT GTACAGCATCGTGCGCCGTG CCGACCGCGC
AGCCGTGCCC ATCGTCAACC TCAAGGACGA GCTGCTGTTT
CCCAGCTGGG AGGCTCTGTT CTCAGGCTCT GAGGGTCCGC
TGAAGCCCGG GGCACGCAT CTTCTCCTTTG ACGGCAAGGA
CGTCCTGAGG CACCCCACCT GGCCCCAGAA GAGCGTGTGG
CATGGCTCGG ACCCCAACGG GCGCAGGCTG ACCGAGAGCT
ACTGTGAGAC GTGGCGGACGGAGGCTCCCT CGGCCACGGG
CCAGGCCTCC TCGCTGCTGG GGGGCAGGCT CCTGGGGCAG
AGTGCCGCGA GCTGCCATCA CGCCTACATC GTGCTCTGCA
TTGAGAACAG CTTCATGACTGCCTCCAAG

### IGFBP-1

IGFBP-1 is a member of the insulin-like growth factor binding protein (IGFBP) family and encodes a protein with an IGFBP domain and a thyroglobulin type-I domain. The protein binds both insulin-like growth factors (IGFs) I and II and circulates in the plasma. Binding of this protein prolongs the half-life of the IGFs and alters their interaction with cell surface receptors.

An amino acid sequence for human IGFBP-1 is publically available in the UniProt database under accession number P08833 (SEQ ID NO: 7) and is as follows:

MSEVPVARVWLVLLLLTVQVGVTAGAPWQCAPCSAEKLALCPP
VSASCSEVTRSAGCGCCPMCALPLGAACGVATARCARGLSCRA
LPGEQQPLHALTRGQGACVQESDASAPHAAEAGSPESPESTEI
TEEELLDNFHLMAPSEEDHSILWDAISTYDGSKALHVTNIKKW
KEPCRIELYRVVESLAKAQETSGEEISKFYLPNCNKNGFYHSR
QCETSMDGEAGLCWCVYPWNGKRIPGSPEIRGDPNCQIYFNVQ
N

A nucleotide sequence that encodes human IGFBP-1 is publically available in the GenBank database under accession number NM_000596.2 (SEQ ID NO: 8) and is as follows (the start and stop codons are underlined and bolded):

GGTGCACTAGCAAAACAAACTTATTTTGAACACTC
AGCTCCTAGCGTGCGGCGCTGCCAATCATTAACCT
CCTGGTGCAAGTGGCGCGGCCTGTGCCCTTTATAA
GGTGCGCGCTGTGTCCAGCGAGCATCGGCCACCGC
CATCCCATCCAGCGAGCATCTGCCGCCGCGCCGCC
GCCACCCTCCCAGAGAGCACTGGCCACCGCTCCAC
CATCACTTGCCCAGAGTTTGGGCCACCGCCCGCCG
CCACCAGCCCAGAGAGCATCGGCCCCTGTCTGCTG
CTCGCGCCTGGAGATGTCAGAGGTCCCCGTTGCTC
GCGTCTGGCTGGTACTGCTCCTGCTGACTGTCCAG
GTCGGCGTGACAGCCGGCGCTCCGTGGCAGTGCGC
GCCCTGCTCCGCCGAGAAGCTCGCGCTCTGCCCGC
CGGTGTCCGCCTCGTGCTCGGAGGTCACCCGGTCC
GCCGGCTGCGGCTGTTGCCCGATGTGCGCCCTGCC
TCTGGGCGCCGCGTGCGGCGTGGCGACTGCACGCT
GCGCCCGGGGACTCAGTTGCCGCGCGCTGCCGGGG
GAGCAGCAACCTCTGCACGCCCTCACCCGCGGCCA
AGGCGCCTGCGTGCAGGAGTCTGACGCCTCCGCTC
CCCATGCTGCAGAGGCAGGGAGCCCTGAAAGCCCA
GAGAGCACGGAGATAACTGAGGAGGAGCTCCTGGA
TAATTTCCATCTGATGGCCCCTTCTGAAGAGGATC
ATTCCATCCTTTGGGACGCCATCAGTACCTATGAT
GGCTCGAAGGCTCTCCATGTCACCAACATCAAAAA
ATGGAAGGAGCCCTGCCGAATAGAACTCTACAGAG
TCGTAGAGAGTTTAGCCAAGGCACAGGAGACATCA
GGAGAAGAAATTTCCAAATTTTACCTGCCAAACTG
CAACAAGAATGGATTTTATCACAGCAGACAGTGTG
AGACATCCATGGATGGAGAGGCGGGACTCTGCTGG
TGCGTCTACCCTTGGAATGGGAAGAGGATCCCTGG
GTCTCCAGAGATCAGGGGAGACCCCAACTGCCAGA
TATATTTTAATGTACAAAACTGAAACCAGATGAAA
TAATGTTCTGTCACGTGAAATATTTAAGTATATAG
TATATTTATACTCTAGAACATGCACATTTATATAT
ATATGTATATGTATATATATATAGTAACTACTTTT
T.ATACTCCATACATAACTTGATATAGAAAGCTGT
TTATTTATTCACTGTAAGTTTATTTTTTCTACACA
GTAAAAACTTGTACTATGTTAATAACTTGTCCTAT
GTCAATTTGTATATCATGAAACACTTCTCATCATA
TTGTATGTAAGTAATTGCATTTCTGCTCTTCCAAA
GCTCCTGCGTCTGTTTTTAAAGAGCATGGAAAAAT
ACTGCCTAGAAAATGCAAAATGAAATAAGAGAGAG
TAGTTTTTCAGCTAGTTTGAAGGAGGACGGTTAAC
TTGTATATTCCACCATTCACATTTGATGTACATGT
GTAGGGAAAGTTAAAAGTGTTGATTACATAATCAA
AGCTACCTGTGGTGATGTTGCCACCTGTTAAAATG
TACACTGGATATGTTGTTAAACACGTGTCTATAAT
GGAAACATTTACAATAAATATTCTGCATGGAAATA
CTGTTAAAAAAAAAAA

### HTRA1

HTRA1 is a serine protease with a variety of targets, including extracellular matrix proteins such as fibronectin. HTRA1-generated fibronectin fragments further induce synovial cells to up-regulate matrix metalloproteinase-1 (MMP1) and matrix metalloproteinase-3 (MMP3) production. HTRA1 may also degrade proteoglycans, such as aggrecan, decorin and fibromodulin. Through cleavage of proteoglycans, HTRA1 may release soluble fibroblast growth factor (FGF)-glycosaminoglycan complexes that promote the range and intensity of FGF signals in the extracellular space. HTRA1 is also thought to regulate the availability of insulin-like growth factors (IGFs) by cleaving IGF-binding proteins. HTRA1 is further believed to inhibit signaling mediated by transforming growth factor beta (TGF-β) family members. This activity requires the integrity of the catalytic site, although it is unclear whether TGF-β proteins are themselves degraded. By acting on TGF-β signaling, HTRA1 may regulate many physiological processes, including retinal angiogenesis and neuronal survival and maturation during development. Intracellularly, HTRA1 degrades Tuberous Sclerosis Complex 2 (TSC2), leading to the activation of TSC2 downstream targets.

An amino acid sequence for human HTRA1 is publically available in the UniProt database under accession number Q92743 (SEQ ID NO: 9) and is as follows:

MQIPRAALLPLLLLLLAAPASAQLSRAGRSAPLAA
GCPDRCEPARCPPQPEHCEGGRARDACGCCEVCGA
PEGAACGLQEGPCGEGLQCVVPFGVPASATVRRRA
QAGLCVCASSEPVCGSDANTYANLCQLRAASRRSE
RLHRPPVIVLQRGACGQGQEDPNSLRHKYNFIADV
VEKIAPAVVHIELFRKLPFSKREVPVASGSGFIVS
EDGLIVTNAHVVTNKHRVKVELKNGATYEAKIKDV
DEKADIALIKIDHQGKLPVLLLGRSSELRPGEFVV
AIGSPFSLQNTVTTGIVSTTQRGGKELGLRNSDMD
YIQTDAIINYGNSGGPLVNLDGEVIGINTLKVTAG
ISFAIPSDKIKKFLTESHDRQAKGKAITKKKYIGI
RMMSLTSSKAKELKDRHRDFPDVISGAYIIEVIPD
TPAEAGGLKENDVIISINGQSVVSANDVSDVIKRE
STLNMVVRRGNEDIMITVIPEEIDP

In the sequence shown above, positions 1-22 (SEQ ID NO: 10) correspond to the signal peptide, positions 204-364 (SEQ ID NO: 11) correspond to a serine protease domain.

A nucleotide sequence that encodes human HTRA1 is publically available in the GenBank database under accession number NM_002775.4 (SEQ ID NO: 12) and is as follows (the start and stop codons are underlined and bolded):

CAATGGGCTGGGCCGCGCGGCCGCGCGCACTCGCA
CCCGCTGCCCCCGAGGCCCTCCTGCACTCTCCCCG
GCGCCGCTCTCCGGCCCTCGCCCTGTCCGCCGCCA
CCGCCGCCGCCGCCAGAGTCGCCATGCAGATCCCG
CGCGCCGCTCTTCTCCCGCTGCTGCTGCTGCTGCT
GGCGGCGCCCGCCTCGGCGCAGCTGTCCCGGGCCG
GCCGCTCGGCGCCTTTGGCCGCCGGGTGCCCAGAC
CGCTGCGAGCCGGCGCGCTGCCCGCCGCAGCCGGA
GCACTGCGAGGGCGGCCGGGCCCGGGACGCGTGCG
GCTGCTGCGAGGTGTGCGGCGCGCCCGAGGGCGCC
GCGTGCGGCCTGCAGGAGGGCCCGTGCGGCGAGGG
GCTGCAGTGCGTGGTGCCCTTCGGGGTGCCAGCCT
CGGCCACGGTGCGGCGGCGCGCGCAGGCCGGCCTC
TGTGTGTGCGCCAGCAGCGAGCCGGTGTGCGGCAG
CGACGCCAACACCTACGCCAACCTGTGCCAGCTGC
GCGCCGCCAGCCGCCGCTCCGAGAGGCTGCACCGG
CCGCCGGTCATCGTCCTGCAGCGCGGAGCCTGCGG
CCAAGGGCAGGAAGATCCCAACAGTTTGCGCCATA
AATATAACTTTATCGCGGACGTGGTGGAGAAGATC
GCCCCTGCCGTGGTTCATATCGAATTGTTTCGCAA
GCTTCCGTTTTCTAAACGAGAGGTGCCGGTGGCTA
GTGGGTCTGGGTTTATTGTGTCGGAAGATGGACTG
ATCGTGACAAATGCCCACGTGGTGACCAACAAGCA
CCGGGTCAAAGTTGAGCTGAAGAACGGTGCCACTT
ACGAAGCCAAAATCAAGGATGTGGATGAGAAAGCA
GACATCGCACTCATCAAAATTGACCACCAGGGCAA
GCTGCCTGTCCTGCTGCTTGGCCGCTCCTCAGAGC
TGCGGCCGGGAGAGTTCGTGGTCGCCATCGGAAGC
CCGTTTTCCCTTCAAAACACAGTCACCACCGGGAT
CGTGAGCACCACCCAGCGAGGCGGCAAAGAGCTGG
GGCTCCGCAACTCAGACATGGACTACATCCAGACC
GACGCCATCATCAACTATGGAAACTCGGGAGGCCC
GTTAGTAAACCTGGACGGTGAAGTGATTGGAATTA
ACACTTTGAAAGTGACAGCTGGAATCTCCTTTGCA
ATCCCATCTGATAAGATTAAAAAGTTCCTCACGGA
GTCCCATGACCGACAGGCCAAAGGAAAAGCCATCA
CCAAGAAGAAGTATATTGGTATCCGAATGATGTCA
CTCACGTCCAGCAAAGCCAAAGAGCTGAAGGACCG
GCACCGGGACTTCCCAGACGTGATCTCAGGAGCGT
ATATAATTGAAGTAATTCCTGATACCCCAGCAGAA
GCTGGTGGTCTCAAGGAAAACGACGTCATAATCAG
CATCAATGGACAGTCCGTGGTCTCCGCCAATGATG
TCAGCGACGTCATTAAAAGGGAAAGCACCCTGAAC
ATGGTGGTCCGCAGGGGTAATGAAGATATCATGAT
CACAGTGATTCCCGAAGAAATTGACCCATAGGCAG
AGGCATGAGCTGGACTTCATGTTTCCCTCAAAGAC
TCTCCCGTGGATGACGGATGAGGACTCTGGGCTGC
TGGAATAGGACACTCAAGACTTTTGACTGCCATTT
TGTTTGTTCAGTGGAGACTCCCTGGCCAACAGAAT
CCTTCTTGATAGTTTGCAGGCAAAACAAATGTAAT
GTTGCAGATCCGCAGGCAGAAGCTCTGCCCTTCTG
TATCCTATGTATGCAGTGTGCTTTTTCTTGCCAGC
TTGGGCCATTCTTGCTTAGACAGTCAGCATTTGTC
TCCTCCTTTAACTGAGTCATCATCTTAGTCCAACT
AATGCAGTCGATACAATGCGTAGATAGAAGAAGCC
CCACGGGAGCCAGGATGGGACTGGTCGTGTTTGTG
CTTTTCTCCAAGTCAGCACCCAAAGGTCAATGCAC
AGAGACCCCGGGTGGGTGAGCGCTGGCTTCTCAAA
CGGCCGAAGTTGCCTCTTTTAGGAATCTCTTTGGA
ATTGGGAGCACGATGACTCTGAGTTTGAGCTATTA
AAGTACTTCTTACACATTGCAAAAAAAAAAAAAAA
AAA

### Exemplary SVDs and the Treatment Thereof

Aspects of the present subject matter relate to the treatment of SVDs. Non-limiting examples of SVDs are discussed below.

### Cerebral Small Vessel Disease

As used herein, the term “cerebral small vessel disease” or “cerebral SVD” refers to a group of pathological processes with various aetiologies that affect the small arteries, arterioles, venules, and capillaries of the brain. See, e.g., Pantoni (2010) Lancet Neurol, 9(7):689-701, the entire contents of which are incorporated herein by reference. Age-related and hypertension-related SVDs and cerebral amyloid angiopathy are the most common forms. The consequences of small vessel disease on the brain parenchyma are mainly lesions located in the subcortical structures such as lacunar infarcts, white matter lesions, large hemorrhages, and microbleeds. Small vessel disease has an important role in cerebrovascular disease and is a leading cause of cognitive decline and functional loss in the elderly.

Cerebral SVD may lead to vascular dementia (also known as vascular cognitive impairment). In vascular dementia, changes in thinking skills sometimes occur suddenly following strokes that block major brain blood vessels. See, e.g., Alzheimer's Association, Alzheimer's & Dementia, available at www.alz.org/dementia/vascular-dementia-symptoms.asp, the entire contents of which are incorporated herein by reference. Thinking problems also may begin as mild changes that worsen gradually as a result of multiple minor strokes or other conditions that affect smaller blood vessels, leading to cumulative damage. Symptoms can vary widely, depending on the severity of the blood vessel damage and the part of the brain affected. Memory loss may or may not be a significant symptom depending on the specific brain areas where blood flow is reduced. Vascular dementia symptoms may be most obvious when they happen soon after a major stroke. Sudden post-stroke changes in thinking and perception may include, e.g., (i) confusion; (ii) disorientation; (iii) trouble speaking or understanding speech; and/or (iv) vision loss. These changes may happen at the same time as stroke symptoms such as a sudden headache, difficulty walking, or numbness or paralysis on one side of the face or the body.

Multiple small strokes or other conditions that affect blood vessels and nerve fibers deep inside the brain may cause more gradual thinking changes as damage accumulates. Common early signs of widespread small vessel disease include impaired planning and judgment; uncontrolled laughing and crying; declining ability to pay attention; impaired function in social situations; and difficulty finding the right words.

The present subject matter provides methods for treating each subtype, symptom, and/or complication of cerebral SVD.

### HTRA1-Associated Small Vessel Disease

As used herein, an “HTRA1-associated small vessel disease” or HTRA1-associated SVD is a SVD that results from a dominant HTRA1 mutation. In various embodiments, a subject is heterozygous for the mutation. Descriptions of exemplary heterozygous mutations of the HTRA1 gene in patients with familial cerebral small vessel disease are included in Donato et al. 2017 “Heterozygous mutations of HTRA1 gene in patients with familial cerebral small vessel disease” CNS Neurosci Ther. 23(9):759-765; and Verdura et al. (2015) “Heterozygous HTRA1 mutations are associated with autosomal dominant cerebral small vessel disease” Brain 138; 2347-2358, the entire contents of each of which are incorporated herein by reference.

Cerebral Autosomal Recessive Arteriopathy with Subcortical Infarcts and Leukoencephalopathy

Cerebral autosomal recessive arteriopathy with subcortical infarcts and leukoencephalopathy, commonly known as CARASIL, is an inherited condition that causes stroke and other impairments. As its name suggests, this condition is inherited in an autosomal recessive pattern. Autosomal recessive inheritance means both copies of the gene in each cell have mutations. The parents of an individual with an autosomal recessive condition each carry one copy of the mutated gene, but they typically do not show signs and symptoms of the condition. See, e.g., the U.S. National Library of Medicine Genetics Home Reference, CARASIL, available at ghr.nlm.nih.gov/condition/cerebral-autosomal-recessive-arteriopathy-with-subcortical-infarcts-and-leukoencephalopathy#inheritance, the entire contents of which are incorporated herein by reference.

Abnormalities affecting the brain and other parts of the nervous system become apparent in an affected person's twenties or thirties. Often, muscle stiffness (spasticity) in the legs and problems with walking are the first signs of the disorder. About half of affected individuals have a stroke or similar episode before age 40. As the disease progresses, most people with CARASIL also develop mood and personality changes, a decline in thinking ability (dementia), memory loss, and worsening problems with movement.

Other characteristic features of CARASIL include premature hair loss (alopecia) and attacks of low back pain. The hair loss often begins during adolescence and is limited to the scalp. Back pain, which develops in early to mid-adulthood, results from the breakdown (degeneration) of the discs that separate the bones of the spine (vertebrae) from one another.

The signs and symptoms of CARASIL worsen slowly with time. Over the course of several years, affected individuals become less able to control their emotions and communicate with others. They increasingly require help with personal care and other activities of daily living; after a few years, they become unable to care for themselves. Most affected individuals die within a decade after signs and symptoms first appear, although few people with the disease have survived for 20 to 30 years.

The present subject matter provides methods for treating each subtype, symptom, and/or complication of CARASIL.

Cerebral Autosomal-Dominant Arteriopathy with Subcortical Infarcts and Leukoencephalopathy

Cerebral autosomal dominant arteriopathy with subcortical infarcts and leukoencephalopathy, usually called CADASIL, is an inherited condition that causes stroke and other impairments. This condition affects blood flow in small blood vessels, particularly cerebral vessels within the brain. The muscle cells surrounding these blood vessels (vascular smooth muscle cells) are abnormal and gradually die. In the brain, the resulting blood vessel damage (arteriopathy) can cause migraines, often with visual sensations or auras, or recurrent seizures (epilepsy). See, e.g., the U.S. National Library of Medicine Genetics Home Reference, CADASIL, available at ghr.nlm.nih.gov/condition/cerebral-autosomal-dominant-arteriopathy-with-subcortical-infarcts-and-leukoencephalopathy#genes, the entire contents of which are incorporated herein by reference.

Damaged blood vessels reduce blood flow and can cause areas of tissue death (infarcts) throughout the body. An infarct in the brain can lead to a stroke. In individuals with CADASIL, a stroke can occur at any time from childhood to late adulthood, but typically happens during mid-adulthood. People with CADASIL often have more than one stroke in their lifetime. Recurrent strokes can damage the brain over time. Strokes that occur in the subcortical region of the brain, which is involved in reasoning and memory, can cause progressive loss of intellectual function (dementia) and changes in mood and personality.

Many people with CADASIL also develop leukoencephalopathy, which is a change in a type of brain tissue called white matter that can be seen with magnetic resonance imaging (MRI).

The age at which the signs and symptoms of CADASIL first begin varies greatly among affected individuals, as does the severity of these features.

CADASIL is not associated with the common risk factors for stroke and heart attack, such as high blood pressure and high cholesterol, although some affected individuals might also have these health problems.

Mutations in the NOTCH3 gene cause CADASIL. One copy of the altered NOTCH3 gene in each cell is sufficient to cause the disorder. The NOTCH3 gene provides instructions for producing the NOTCH3 receptor protein, which is important for the normal function and survival of vascular smooth muscle cells. When certain molecules attach (bind) to NOTCH3 receptors, the receptors send signals to the nucleus of the cell. These signals then turn on (activate) particular genes within vascular smooth muscle cells.

NOTCH3 gene mutations lead to the production of an abnormal NOTCH3 receptor protein that impairs the function and survival of vascular smooth muscle cells. Disruption of NOTCH3 functioning can lead to the self-destruction (apoptosis) of these cells. In the brain, the loss of vascular smooth muscle cells results in blood vessel damage that can cause the signs and symptoms of CADASIL.

Prior to the present invention, no specific treatment was available for CADASIL. However, anti-platelet agents such as aspirin, dipyridamole, ticlopidine, and clopidogrel are used to slow down the disease and help prevent strokes. Aspects of the present invention relate to administering an anti-platelet agent to a subject who is diagnosed with or determined to be at risk of developing CADASIL. In some embodiments, the subject receives therapy for primary or secondary prevention of stroke and myocardial infarction. Risk-reduction measures in primary stroke prevention may include the use of antihypertensive medications; platelet antiaggregants; 3-hydroxy-3-methylglutaryl coenzyme A (HMG-CoA) reductase inhibitors (statins); smoking cessation; dietary intervention; weight loss; and exercise. Secondary prevention may include the use of antiaggregants (aspirin, clopidogrel, extended-release dipyridamole, ticlopidine), cholesterol-reducing medications, and/or blood pressure-lowering medications, as well as the cessation of cigarette smoking, improving the diet (e.g., reducing red meat consumption and/or increasing vegetable consumption), and increased exercise.

The present subject matter provides methods for treating each subtype, symptom, and/or complication of CADASIL.

### NOTCH3 Loss of Function-Associated Small Vessel Disease

NOTCH3 loss of function mutations cause an SVD phenotype strikingly similar to CADASIL but with key differences including the lack of accumulation of the NOTCH3 extracellular domain and the lack of GOM deposits. Typical mutations include changes leading to NOTCH3 frame shifts, premature stop codons, or splicing defects. Partial or complete gene deletions or promoter or enhancer mutations leading to lower than normal NOTCH3 expression are also included. It has been reported that in some patients, typical CADASIL mutations also lead to NOTCH3 loss of function and in these, NOTCH3 loss of function contributes to SVD pathology. In most cases, patients with NOTCH3 loss of function are heterozygotes although a homozygote patient has been reported with earlier age at onset of SVD. This indicates that NOTCH3 is haploinsufficient in humans because one wild-type copy of the gene is not sufficient to produce a wild-type phenotype. Conditions that may indirectly lead to a decrease in NOTCH3 expression or function in the absence of mutations include cardiovascular, metabolic disease, disease, environmental factor, and aging.

### Age-Related Macular Degeneration

Age-related macular degeneration (AMD) is an eye disease that is a leading cause of vision loss in older people in developed countries. The vision loss usually becomes noticeable in a person's sixties or seventies and tends to worsen over time. See, e.g., the U.S. National Library of Medicine Genetics Home Reference, Age-Related Macular Degeneration, available at ghr.nlm.nih.gov/condition/age-related-macular-degeneration, the entire contents of which are incorporated herein by reference.

AMD mainly affects central vision, which is needed for detailed tasks such as reading, driving, and recognizing faces. The vision loss in this condition results from a gradual deterioration of light-sensing cells in the tissue at the back of the eye that detects light and color (the retina). Specifically, age-related macular degeneration affects a small area near the center of the retina, called the macula, which is responsible for central vision. Side (peripheral) vision and night vision are generally not affected.

There are two major types of AMD, known as the dry form and the wet form. The dry form is much more common, accounting for 85 to 90 percent of all cases of age-related macular degeneration. It is characterized by a buildup of yellowish deposits called drusen beneath the retina and slowly progressive vision loss. The condition typically affects vision in both eyes, although vision loss often occurs in one eye before the other.

The wet form of AMD is associated with severe vision loss that can worsen rapidly. This form of the condition is characterized by the growth of abnormal, fragile blood vessels underneath the macula. These vessels leak blood and fluid, which damages the macula and makes central vision appear blurry and distorted.

AMD results from a combination of genetic and environmental factors. Many of these factors have been identified, but some remain unknown.

Without wishing to be bound by any scientific theory, changes in many genes may be risk factors for age-related macular degeneration. The best-studied of these genes are involved in a part of the body's immune response known as the complement system. This system is a group of proteins that work together to destroy foreign invaders (such as bacteria and viruses), trigger inflammation, and remove debris from cells and tissues. Genetic changes in and around several complement system genes, including the complement factor H (CFH) gene, contribute to a person's risk of developing age-related macular degeneration. It is unclear how these genetic changes are related to the retinal damage and vision loss characteristic of this condition. Changes on the long (q) arm of chromosome 10 in a region known as 10q26 are also associated with an increased risk of age-related macular degeneration. The 10q26 region contains two genes of interest, age-related maculopathy susceptibility 2 (ARMS2) and high-temperature requirement A serine peptidase 1 (HTRA1). Changes in both genes have been studied as possible risk factors for the disease. However, because the two genes are so close together, it is difficult to tell which gene is associated with age-related macular degeneration risk, or whether increased risk results from variations in both genes. An estimated 15 to 20 percent of people with age-related macular degeneration have at least one first-degree relative (such as a sibling) with the condition. Other genes that are associated with age-related macular degeneration include genes involved in transporting and processing high-density lipoprotein (HDL) and genes that have been associated with other forms of macular disease.

Nongenetic factors that contribute to the risk of age-related macular degeneration are also known. Age appears to be the most important risk factor; the chance of developing the condition increases significantly as a person gets older. Smoking is another established risk factor for age-related macular degeneration.

Aspects of the present subject matter relate to administering a treatment for AMD to a subject who is diagnosed with or determined to be at risk of developing AMD. In some embodiments, the subject is administered a statin. In some embodiments relating to neovascular AMD, the subject is administered an antiangiogenic steroid such as anecortave acetate or triamcinolone acetonide. In various embodiments relating to wet AMD, the subject can be treated with laser coagulation or a medication that stops and sometimes reverses the growth of blood vessels. In certain embodiments, the subject is treated with bevacizumab, ranibizumab, pegaptanib, or aflibercept. In some embodiments, photodynamic therapy is administered to the subject. For example, the drug verteporfin is administered intravenously and light of a certain wavelength (e.g., 689 nm) is then applied to the abnormal blood vessels, which activates the verteporfin to destroy the vessels.

The present subject matter provides diagnostic, prognostic, treatment, and monitoring methods, as well as related compositions, kits, and systems, for each subtype, symptom, and/or complication of AMD.

### Retinopathy

Retinopathy is persistent or acute damage to the retina of the eye. Ongoing inflammation and vascular remodeling may occur over periods of time where the patient is not fully aware of the extent of the disease. Frequently, retinopathy is an ocular manifestation of systemic disease as seen in diabetes or hypertension. Diabetic retinopathy is the leading cause of blindness in working-aged people.

Causes of retinopathy include but are not limited to: (i) diabetes mellitus, which can cause diabetic retinopathy; (ii) arterial hypertension, which can cause hypertensive retinopathy; (iii) retinopathy of prematurity due to prematurity of a newborn (under the 9 months of human pregnancy); (iv) radiation retinopathy due to exposure to ionizing radiation; (v) solar retinopathy due to direct sunlight exposure; (vi) sickle cell disease; (vii) retinal vascular disease such as retinal vein or artery occlusion; (viii) trauma, especially to the head, and several diseases may cause Purtscher's retinopathy; and (ix) hyperviscosity-related retinopathy as seen in disorders which cause paraproteinemia.

Many types of retinopathy are proliferative, most often resulting from neovascularization or blood vessel overgrowth. Angiogenesis is the hallmark precursor that may result in blindness or severe vision loss, particularly if the macula becomes affected. Retinopathy may also be a symptom or complication of a ciliopathic genetic disorder such as Alström syndrome or Bardet-Biedl syndrome.

Aspects of the present subject matter relate to administering a treatment for retinopathy to a subject who is diagnosed with or determined to be at risk of developing retinopathy. Treatment may include laser therapy to the retina and/or the administration of a vascular endothelial growth factor (VEGF) inhibitor.

The present subject matter provides methods for the treatment of each subtype, symptom, and/or complication of retinopathy.

### Microangiopathy

Microangiopathy (or microvascular disease, or small vessel disease) is an angiopathy (i.e. disease of blood vessels) affecting small blood vessels in the body. The condition can occur in any organ of the body. One cause of microangiopathy is long-term diabetes mellitus. In this case, high blood glucose levels cause the endothelial cells lining the blood vessels to take in more glucose than normal (these cells do not depend on insulin). They then form more glycoproteins on their surface than normal, and also cause the basement membrane in the vessel wall to grow abnormally thicker and weaker. Mural cell loss is also a hallmark of microangiopathy and is associated with hyperglycemia. Therefore vessels bleed, leak protein, and slow the flow of blood through the body. As a result, some organs and tissues do not get enough blood (carrying oxygen & nutrients) and are damaged, for example, the retina (diabetic retinopathy) or kidney (diabetic nephropathy). Nerves and neurons, if not sufficiently supplied with blood, are also damaged, which leads to loss of function (diabetic neuropathy, especially peripheral neuropathy).

Massive microangiopathy may cause microangiopathic hemolytic anemia (MAHA).

The present subject matter provides methods for the treatment of each subtype, symptom, and/or complication of microangiopathy.

### Nephropathy and Small Vessel Diseases of the Kidney

SVD can occur in the kidneys during or as part of nephropathy. For example, diabetic nephropathy (or diabetic kidney disease) is a progressive kidney disease caused by damage to the capillaries in the kidneys' glomeruli. It is characterized by nephrotic syndrome and diffuse scarring of the glomeruli. It is due to longstanding diabetes mellitus, and is a prime reason for dialysis in many developed countries. It is classified as a small blood vessel complication of diabetes. During its early course, diabetic nephropathy often has no symptoms. Symptoms can take 5 to 10 years to appear after the kidney damage begins. These late symptoms include severe tiredness, headaches, a general feeling of illness, nausea, vomiting, frequent voiding, lack of appetite, itchy skin, and leg swelling.

The present subject matter provides methods for the treatment of each subtype, symptom, and/or complication of nephropathy.

### Proximal 19p13.12 Microdeletion Syndrome

In embodiments, the SVD comprises proximal 19p13.12 microdeletion syndrome. Non-limiting descriptions relating to this syndrome are provided in Huynh et al. (2018) “First prenatal case of proximal 19p13.12 microdeletion syndrome: New insights and new delineation of the syndrome” Eur J Med Genet. S1769-7212(17)30466-4, the entire content of which is incorporated herein by reference.

In certain embodiments, proximal 19p13.12 microdeletion syndrome comprises intellectual disability, facial dysmorphism, and/or branchial arch defects. In some embodiments, proximal 19p13.12 microdeletion syndrome comprises hypertrichosis-synophrys-protruding front teeth. In various embodiments, a subject with proximal 19p13.12 microdeletion syndrome comprises a heterozygous interstitial deletion at 19p13.12 chromosome region. In certain embodiments, the deletion is a deletion of about 350 kb to about 750 kb. In some embodiments, the deletion is a deletion of about 745 kb. In various embodiments, the deletion includes at least a portion of the NOTCH3 gene. In certain embodiments, the deletion includes the entire NOTCH3 gene. In some embodiments, the deletion comprises (e.g., in addition to a mutation in part of all of the NOTCH3 gene) a portion of, or the entirety of any one of, any combination of the following genes: SYDE1, AKAP8, AKAP8L, WIZ and BRD4.

The present subject matter provides methods for the treatment of each subtype, symptom, and/or complication of proximal 19p13.12 microdeletion syndrome.

### Myocardial Ischemia

NOTCH3 deficiency impairs coronary microvascular maturation and reduces cardiac recovery after myocardial ischemia. See, e.g., Tao et al. (2017) “Notch3 deficiency impairs coronary microvascular maturation and reduces cardiac recovery after myocardial ischemia” Int J Cardiol. 2017 Jun. 1; 236:413-422, the entire content of which is incorporated herein by reference. In various embodiments, a subject with myocardial ischemia has myocardial infarction.

In certain embodiments, reduced NOTCH3 results in a reduction of pericytes and small arterioles. In some embodiments, the reduction in pericytes and small arterioles increases the severity of myocardial ischemia, and/or reduces cardiac recovery after myocardial ischemia. In various embodiments, a subject with reduced NOTCH3 function (e.g., due to a mutation) is prone to ischemic injury with larger infarcted size and higher rates of mortality. In certain embodiments, the expression of CXCR-4 and VEGF/Ang-1 is decreased in a subject with reduced NOTCH3 function. In some embodiments, a subject with reduced NOTCH3 function has fewer NG2+/Sca1+ and NG2+/c-kit+ progenitor cells in an ischemic area and exhibits worse cardiac function recovery at 2 weeks after myocardial ischemia compared to a corresponding subject with a normal level of NOTCH3 function. In certain embodiments, a subject with reduced NOTCH3 function has a significant reduction of pericyte/capillary coverage and arteriolar maturation compared to a corresponding subject with a normal level of NOTCH3 function. In various embodiments, a subject with a reduced level of NOTCH3 function and who has had myocardial ischemia has increased intracellular adhesion molecule-2 (ICAM-2) expression and CD11b+ macrophage infiltration into ischemic areas compared to that of a corresponding subject with a normal level of NOTCH3 function. In embodiments, a subject has a NOTCH3 mutation that impairs recovery of cardiac function post-myocardial ischemia by the mechanisms involving the pre-existing coronary microvascular dysfunction conditions, and impairment of pericyte/progenitor cell recruitment and microvascular maturation.

The present subject matter provides methods for the treatment of each subtype, symptom, and/or complication of myocardial ischemia.

### Heart Failure

Heart failure is a chronic, progressive condition in which the heart muscle is unable to pump enough blood through to meet the body's needs for blood and oxygen. Loss of NOTCH3 signaling in vascular smooth muscle cells promotes severe heart failure upon hypertension. See, e.g., Ragot et al., (2016) Hypertension. 68(2):392-400; and the American Heart Association, What is Heart Failure? available at www.heart.org/HEARTORG/Conditions/HeartFailure/AboutHeartFailure/About-Heart-Failure_UCM_002044_Article.jsp #.WM16W_71va8, the entire contents of each of which are incorporated herein by reference.

The heart tries to make up for this by enlarging, developing more muscle mass, and/or pumping faster. When the heart chamber enlarges, it stretches more and can contract more strongly, so it pumps more blood. With an enlarged heart, the body starts to retain fluid, the lungs get congested with fluid and the heart begins to beat irregularly. An increase in muscle mass occurs because the contracting cells of the heart get bigger. This lets the heart pump more strongly, at least initially. Increased heartrate helps to increase the heart's output.

The body also tries to compensate in other ways: (i) The blood vessels narrow to keep blood pressure up, trying to make up for the heart's loss of power; and (ii) The body diverts blood away from less important tissues and organs (like the kidneys), the heart and brain.

These temporary measures mask the problem of heart failure, but they do not solve it. Heart failure continues and worsens until these substitute processes no longer work. Eventually the subject experiences the fatigue, breathing problems or other symptoms that usually prompt a trip to the doctor.

Heart failure can involve the heart's left side, right side or both sides. However, it usually affects the left side first.

The present subject matter provides methods for the treatment of each subtype, symptom, and/or complication of heart failure.

### Alagille Syndrome and Familial Tetralogy of Fallot

Alagille syndrome is a genetic disorder that can affect the liver, heart, and other parts of the body. See, e.g., the U.S. National Library of Medicine Genetics Home Reference, Alagille syndrome, available at ghr.nlm.nih.gov/condition/alagille-syndrome, the entire contents of which are incorporated herein by reference.

One of the major features of Alagille syndrome is liver damage caused by abnormalities in the bile ducts. These ducts carry bile (which helps to digest fats) from the liver to the gallbladder and small intestine. In Alagille syndrome, the bile ducts may be narrow, malformed, and reduced in number (bile duct paucity). As a result, bile builds up in the liver and causes scarring that prevents the liver from working properly to eliminate wastes from the bloodstream. Signs and symptoms arising from liver damage in Alagille syndrome may include a yellowish tinge in the skin and the whites of the eyes (jaundice), itchy skin, and deposits of cholesterol in the skin (xanthomas).

Alagille syndrome is also associated with several heart problems, including impaired blood flow from the heart into the lungs (pulmonic stenosis). Pulmonic stenosis may occur along with a hole between the two lower chambers of the heart (ventricular septal defect) and other heart abnormalities. This combination of heart defects is called tetralogy of Fallot.

People with Alagille syndrome may have distinctive facial features including a broad, prominent forehead; deep-set eyes; and a small, pointed chin. The disorder may also affect the blood vessels within the brain and spinal cord (central nervous system) and the kidneys. Affected individuals may have an unusual butterfly shape of the bones of the spinal column (vertebrae) that can be seen in an x-ray.

Problems associated with Alagille syndrome generally become evident in infancy or early childhood. The severity of the disorder varies among affected individuals, even within the same family. Symptoms range from so mild as to go unnoticed to severe heart and/or liver disease requiring transplantation.

Some people with Alagille syndrome may have isolated signs of the disorder, such as a heart defect like tetralogy of Fallot, or a characteristic facial appearance. These individuals do not have liver disease or other features typical of the disorder.

In more than 90 percent of cases, mutations in the JAGGED1 gene cause Alagille syndrome. Another 7 percent of individuals with Alagille syndrome have small deletions of genetic material on chromosome 20 that include the JAG1 gene, which encodes JAGGED1. A few people with Alagille syndrome have mutations in a different gene, called NOTCH2. The JAG1 and NOTCH2 genes provide instructions for making proteins that fit together to trigger interactions called Notch signaling between neighboring cells during embryonic development. This signaling influences how the cells are used to build body structures in the developing embryo. Changes in either the JAG1 gene or NOTCH2 gene probably disrupt the Notch signaling pathway. As a result, errors may occur during development, especially affecting the bile ducts, heart, spinal column, and certain facial features.

The present subject matter provides methods for the treatment of each subtype, symptom, and/or complication of Alagille syndrome and/or familial tetralogy of Fallot.

### Patent Ductus Arteriosus

Patent ductus arteriosus (PDA) is a condition wherein the ductus arteriosus fails to close after birth. Early symptoms are uncommon, but in the first year of life include increased work of breathing and poor weight gain. An uncorrected PDA may lead to congestive heart failure with increasing age.

The ductus arteriosus is a fetal blood vessel that closes soon after birth. In a PDA, the vessel does not close and remains “patent” (open), resulting in irregular transmission of blood between the aorta and the pulmonary artery. PDA is common in newborns with persistent respiratory problems such as hypoxia, and has a high occurrence in premature newborns. Premature newborns are more likely to be hypoxic and have PDA due to underdevelopment of the heart and lungs.

A PDA allows a portion of the oxygenated blood from the left heart to flow back to the lungs by flowing from the aorta (which has higher pressure) to the pulmonary artery. If this shunt is substantial, the neonate becomes short of breath: the additional fluid returning to the lungs increases lung pressure, which in turn increases the energy required to inflate the lungs. This uses more calories than normal and often interferes with feeding in infancy. This condition, as a constellation of findings, is called congestive heart failure.

In some congenital heart defects (such as in transposition of the great vessels) a PDA may need to remain open, as it is the only way that oxygenated blood can mix with deoxygenated blood. In these cases, prostaglandins are used to keep the DA open until surgical correction of the heart defect is completed.

PDA is associated with NOTCH3 loss of function. See, e.g., Baeten et al., (2015) Genesis 53(12):738-48, the entire content of which is incorporated herein by reference.

The present subject matter provides methods for the treatment of each subtype, symptom, and/or complication of PDA.

### Cerebral Cavernous Malformations

Cerebral cavernous malformations are collections of small blood vessels (capillaries) in the brain that are enlarged and irregular in structure. These capillaries have abnormally thin walls, and they lack other support tissues, such as elastic fibers, which normally make them stretchy. See, e.g., the U.S. National Library of Medicine Genetics Home Reference, Cerebral Cavernous Malformation, available at ghr.nlm.nih.gov/condition/cerebral-cavernous-malformation, the entire contents of which are incorporated herein by reference. As a result, the blood vessels are prone to leakage, which can cause the health problems related to this condition. Cavernous malformations can occur anywhere in the body, but usually produce serious signs and symptoms only when they occur in the brain and spinal cord (which are described as cerebral).

Approximately 25 percent of individuals with cerebral cavernous malformations never experience any related health problems. Other people with this condition may experience serious signs and symptoms such as headaches, seizures, paralysis, hearing or vision loss, and bleeding in the brain (cerebral hemorrhage). Severe brain hemorrhages can result in death. The location and number of cerebral cavernous malformations determine the severity of this disorder. These malformations can change in size and number over time.

There are two forms of the condition: familial and sporadic. The familial form is passed from parent to child, and affected individuals typically have multiple cerebral cavernous malformations. The sporadic form occurs in people with no family history of the disorder. These individuals typically have only one malformation.

Defective NOTCH3 signaling is associated with cerebral cavernous malformations. See, e.g., Schultz et al. (2015) Stroke 46(5):1337-43, the entire content of which is incorporated herein by reference.

The present subject matter provides methods for the treatment of each subtype, symptom, and/or complication of cerebral cavernous malformation.

### Lacunar Strokes and Hemorrhagic Strokes

As disclosed herein, the consequences of SVD on the brain parenchyma are mainly lesions located in the subcortical structures such as lacunar infarcts (also termed “lacunar strokes”), white matter lesions, large hemorrhages, and microbleeds. Strokes, such as lacunar strokes and hemorrhagic strokes, are signs of (e.g., may result from) a SVD. A lacunar stroke is the most common type of stroke, and results from the occlusion of one or more small penetrating arteries that provide blood to the brain's deep structures. In some embodiments, a lacunar stroke comprises a small infarct (e.g., 2-20 mm in diameter) in the deep cerebral white matter, basal ganglia, or pons, presumed to result from the occlusion of a single small perforating artery supplying the subcortical areas of the brain. Hemorrhagic strokes (bleeds) result from a weakened vessel that ruptures and bleeds into the surrounding brain. Pericytes have been reported to play different roles during the different phases of ischemic stroke (e.g., lacunar stroke). See, e.g., Yang et al. (2017), Curr Neuropharmacol 15(6): 892-905, the entire content of which is incorporated herein by reference. In some embodiments, pericyte constriction and death may be a cause of the no-reflow phenomenon in brain capillaries during the hyperacute phase of stroke. In certain embodiments, during the acute phase, pericytes detach from microvessels and participate in inflammatory-immunological response, resulting in blood brain barrier (BBB) damage and brain edema. In various embodiments, pericytes are neuroprotective by protecting endothelium, stabilizing BBB and releasing neurotrophins. In some embodiments, pericytes contribute to angiogenesis and neurogenesis, and thereby promote neurological recovery during the recovery phase of stroke.

In certain embodiments, a subject with a SVD has more difficulty recovering from a lacunar stroke compared to a subject without SVD. In some embodiments, a subject with a SVD has more difficulty recovering from a hemorrhagic stroke compared to a subject without SVD. In various embodiments, a treatment herein improves (e.g., the rate or degree of) treatment in a subject with a SVD who has had a lacunar stroke or a hemorrhagic stroke. In certain embodiments, a treatment herein reduces the likelihood that a subject who has a SVD will have a lacunar stroke or a hemorrhagic stroke following treatment. In various embodiments, NOTCH3 signaling manipulation (e.g., increasing NOTCH3 signaling) can stabilize mural cells after stroke (e.g., during the different phases after stroke).

The present subject matter provides methods for the treatment of each subtype, symptom, and/or complication of lacunar strokes and hemorrhagic strokes.

### Subjects at Risk of Developing a Small Vessel Disease or a Symptom or Complication of a Small Vessel Disease

Aspects of the present subject matter relate to inhibiting or preventing a SVD (or a complication or symptom thereof) in a subject who is at risk of developing the SVD (or a symptom or complication thereof). In some embodiments, a subject at risk of developing an SVD or a symptom or complication thereof is administered a therapeutic treatment for the SVD prior to the subject's diagnosis or perception of the SVD or a symptom or complication of the SVD.

Risk factors may vary from SVD to SVD. However, a subject may generally be considered to be at risk of suffering from a SVD or a symptom or complication thereof if the subject has at least 1 grandparent, parent, aunt, uncle, cousin, and/or sibling who suffers from the SVD or the symptom or complication thereof. Additional non-limiting examples of risk factors for SVDs are discussed below.

Cerebral SVD has frequently been found on computed tomography (CT) and magnetic resonance imaging (MRI) scans of elderly people. See, e.g., van Norden et al., (2011) BMC NeurologyBMC series 11:29, the entire contents of which are incorporated herein by reference. In various embodiments, an elderly subject (e.g., a subject who is at least about 70, 75, 80, 85, 90, or 95 years old) is deemed to be at risk of and treated and/or screened for (e.g., using a diagnostic or prognostic method disclosed herein) cerebral SVD and/or a complication or symptom of cerebral SVD. Symptoms and complications of cerebral SVD are disclosed herein and include, e.g., vascular cognitive impairment, hemorrhages and microbleeds, neuropathy, strokes, dementia, and/or parkinsonism. In various embodiments, a subject at risk of developing cerebral SVD or a complication or symptom thereof is a subject who has suffered from at least one stroke. In certain embodiments, a subject is at risk of developing cerebral SVD or a complication or symptom thereof if the subject has hypertension (e.g., a systolic pressure of at least 140 mmHg or a diastolic pressure of at least 90 mmHg) and/or amyloid deposits in the walls of the blood vessels of the central nervous system. There are also hereditary risk factors for cerebral SVD. See, e.g., Plancher et al. Case Rep Neurol. 2015 May-August; 7(2): 142-147, the entire contents of which are incorporated herein by reference. In some embodiments, the subject has a mutated gene that is associated with cerebral SVD. In certain embodiments, a subject is at risk of developing cerebral SVD if the subject has at least 1 grandparent, parent, aunt, uncle, cousin, or sibling who suffers or has suffered from cerebral SVD or a complication or symptom thereof, and/or who has a gene mutation that is associated with cerebral SVD.

In some embodiments, the subject (or at least 1 grandparent, parent, aunt, uncle, cousin, and/or sibling thereof) has a mutation in a COL4A1 gene (which encodes the type IV collagen alpha-1 chain). COL4A1-related brain SVD is part of a group of conditions called the COL4A1-related disorders. See, e.g., the U.S. National Library of Medicine Genetics Home Reference, COL4A1-related brain small-vessel disease, available at ghr.nlm.nih.gov/condition/col4a1-related-brain-small-vessel-disease#genes, the entire contents of which are incorporated herein by reference. The conditions in this group have a range of signs and symptoms that involve fragile blood vessels. COL4A1-related brain small-vessel disease is characterized by weakening of the blood vessels in the brain. Stroke is often the first symptom of this condition, typically occurring in mid-adulthood. In affected individuals, stroke is usually caused by bleeding in the brain (hemorrhagic stroke) rather than a lack of blood flow in the brain (ischemic stroke), although either type can occur. Individuals with this condition are at increased risk of having more than one stroke in their lifetime. People with COL4A1-related brain small vessel disease also have leukoencephalopathy, which is a change in a type of brain tissue called white matter that can be seen with MRI. Affected individuals may also experience seizures and migraine headaches accompanied by visual sensations known as auras. In various embodiments, a subject with a COL4A1 mutation is at risk of and treated and/or screened for (e.g., using a diagnostic or prognostic method disclosed herein) for a symptom or complication such as a ischemic stroke, a hemorrhagic stroke, a migraine, a seizure, leukomalacia, nephropathy, hematuria, chronic muscle cramps, and/or a ocular anterior segment disease.

In some embodiments, a subject is at risk of cerebral SVD (e.g., sporadic cerebral SVD). In certain embodiments, the subject (or at least 1 grandparent, parent, aunt, uncle, cousin, and/or sibling thereof) has a mutation in a COL4A2 gene. COL4A2 is associated with lacunar ischemic stroke and deep intracerebral hemorrhage (ICH). See, e.g., Rannikmae et al. (2017) “COL4A2 is associated with lacunar ischemic stroke and deep ICH: Meta-analyses among 21,500 cases and 40,600 controls” Neurology October 24; 89(17):1829-1839.

In embodiments, subjects at risk of ICH (e.g., deep or lobar ICH) and/or ischemic stroke (IS) (e.g., lacunar, cardioembolic, or large vessel disease) include subjects with a mutation in a COL4A1 or COL4A2 gene.

Subjects at risk of developing CARASIL or CADASIL and/or a symptom or complication thereof include subjects with at least 1 or 2 grandparents, parents, or siblings who suffer from CARASIL, or CADASIL, and/or the symptom or complication thereof. Subjects at risk of developing CARASIL also include subjects who carry a mutation in the HTRA1 gene, or who have a grandparent, parent, or sibling who carries such a mutation. Subjects at risk of developing CADASIL also include subjects who carry a mutation in the NOTCH3 gene, or who have a grandparent, parent, or sibling who carries such a mutation.

Subjects at risk of developing AMD (such as wet or dry AMD) and/or a symptom or complication thereof include subjects with high blood pressure, heart disease, a high-fat diet or one that is low in certain nutrients (such as antioxidants and zinc), obesity, repeated and/or prolonged exposure to ultraviolet (UV) rays from sunlight, or who smoke or have smoked for at least about 1, 5, 10, or more years. Subjects at risk of developing AMD and/or a symptom or complication thereof also include subjects with at least 1 or 2 grandparents, parents, or siblings who suffer from AMD, and/or the symptom or complication thereof. In various embodiments, a subject who carries a mutation in a CFH, ARMS2, HTRA1 gene, or a gene involved in transporting or processing HDL.

Subjects at risk of developing retinopathy include subjects with diabetes, arterial hypertension, sickle cell disease, a retinal vascular disease such as retinal vein or artery occlusion, Alström syndrome, or Bardet-Biedl syndrome. Subjects at risk of developing retinopathy also include premature human newborns (infants about 0, 1, 2, 3, 4, 5, 6, 7, 8, 9, 10 or more weeks old who were born after less than about 9, 8, or 7, months of pregnancy), subjects who have been exposed to ionizing radiation, and subjects whose retinas have been exposed to direct sunlight. In some embodiments, the retinopathy is diabetic retinopathy. Subjects at risk of developing diabetic retinopathy include, e.g., subjects with type 1 or type 2 diabetes. In various embodiments, the retinopathy is proliferative (e.g., proliferative diabetic retinopathy).

Subjects at risk of developing heart failure include subjects with high blood pressure, coronary artery disease, diabetes, sleep apnea, a congenital heart defect, valvular heart disease, or irregular heartbeats. Subjects at risk of heart failure also include alcoholics and former alcoholics, subjects who have used tobacco (e.g., who have smoked cigarettes for at least about 5, 10, 15, or 20 years), subjects who are obese, and subjects who have had a heart attack. Subjects who have taken rosiglitazone, pioglitazone, and nonsteroidal anti-inflammatory drugs (NSAIDs) [e.g., regularly (such as 1, 2, 3, 4, 5, 6, or 7 times per week) for at least about 1, 2, 3, 4, or 5 years] are also at risk for heart failure.

Subjects at risk of developing nephropathy (especially diabetic nephropathy) include subjects who have hyperglycemia, hypertension, at least 1 grandparent, parent, aunt, uncle, cousin, or sibling with nephropathy or hypertension. Additional non-limiting examples include subjects who smoke or have smoked for at least about 1, 5, 10, or more years.

Such subjects may be treated using the methods, agonists, and compositions disclosed herein.

### Gene Therapy

In some embodiments, a gene editing method is used to modulate (e.g., increase) NOTCH3 expression and/or activity. Gene therapy may be used to deliver a nucleic acid polymer into a subject's cells, or in cells to be administered to a subject (e.g., induced pluripotent stem cells or embryonic stem cells), to increase NOTCH3 expression by, e.g. replacing a mutated or defective NOTCH3 gene or a portion thereof, or expressing NOTCH3 (e.g. constitutively or under the direction of a promoter for a specific cell type such as vascular smooth muscle cells) from a construct such as a plasmid, or a virally delivered construct (such as a retroviral construct) or adding one or more extra copies of a full length human wild-type gene.

In some embodiments, a viral construct is delivered using, a retroviral vector, a adeno-associated viral vector, a poxvirus vector, or a non-viral vector.

In certain embodiments, a non-viral vector is used to deliver a construct. Non-viral vectors include naked-DNA and liposomes. In various embodiments, a construct comprises plasmid. Therapeutic genes can be inserted directly into the plasmid, and then this recombinant plasmid can be introduced into cells in a variety of ways. For example, it can be injected directly into targeted tissues as naked-DNA. In certain embodiments, naked DNA (e.g., a plasmid) or a liposome is injected. In some embodiments, particle-mediated gene transfer (‘the gene gun’) is used to deliver a plasmid. The genetic material can be placed in liposomes in order to increase the DNA uptake in cells. In certain embodiments, gene gun delivery comprises micro or nano particles (e.g., gold or tungsten) coated with DNA that are accelerated by either helium pressure or a high-voltage electrical discharge to carry enough energy to penetrate cell membranes.

In some embodiments, a non-viral vector is combined with an agent that facilitates its entry into cells such as a condensing agent. In some embodiments, the vector is condensed with a condensing agent. In certain embodiments, the condensing agent comprises a cationic compound. In various embodiments, the cationic compound comprises a cationic compound. Non-limiting examples of condensing agents include spermidine, spermine, cobalthexamine, PEI, PLL, PAMAM dendrimers, and chitosan. In some embodiments, a complex comprising a non-viral vector and a condensing agent is coated with hydrophilic polyethylene glycol (PEG). In some embodiments, a non-viral vector is complexed with a MNP. Exemplary MNPs for gene delivery are constructed from amphiphilic diblock AB or triblock ABC copolymers where A counts for the hydrophobic micelle-forming segment, B for the cationic nucleic acid-loading segment, and C for hydrophilic micelle-stabilizer segment. Two driving forces are responsible for MNP formation: (i) the hydrophobic interactions between the hydrophobic segments of the amphiphiles due to the reorganization of the surrounding water and (ii) the attractive electrostatic forces that exist between oppositively charged nucleic acids and cationic amphiphiles. Additional non-limiting aspects of MNPs are described in Navarro et al. (2015) Mol Pharm. 12(2): 301-313, the entire content of which is incorporated herein by reference.

In various embodiments, cells (e.g., of a cell line or primary cells in culture) are genetically modified ex vivo and then administered to a subject. In some embodiments, the cells are from the subject. In certain embodiments, the cells are from a donor (e.g., a common donor). In various embodiments, a cell (e.g. primary vascular smooth muscle cells, mural cells, pericytes, as well as others described below), or cell line is modified by transfection using liposomes or by infection using a virus. In some embodiments, the cells comprise pericytes. In certain embodiments, the cells comprise vascular smooth muscle cells. In certain embodiments, the cells comprise perivascular fibroblast-like cells. In various embodiments, the cells are obtained from a donor, e.g., from a donor's blood, bone marrow, or a skin biopsy. In some embodiments, the cells are expanded in vitro. In certain embodiments, the cells are obtained from a donor by obtaining blood (e.g., peripheral blood mononuclear cells) or fibroblasts that are then treated to become induced pluripotent cells, which are then differentiated to become pericytes, vascular smooth muscle cells, or perivascular fibroblast-like cells. In some embodiments, the cells are obtained from a universal donor or from embryonic or induced pluripotent cells, and then differentiated in vitro prior to manipulation. In certain embodiments, genetically modified cells are administered via infusion into the blood or into cerebral ventricles. In various embodiments, genetically modified cells are surgically transplanted into the brain. In some embodiments, the cells have a tropism for vessels and home there.

In some embodiments, gene therapy comprises the transplantation of a stem cell has been obtained from a subject and then genetically modified. For example the cell may have been modified to increase NOTCH3 expression from an exogenous construct or by replacing or reverse-mutating a mutated NOTCH3 gene.

Non-limiting examples of gene editing systems useful in various embodiments include the clustered regularly interspaced short palindromic repeat (CRISPR)-Cas system; zinc finger nuclease (ZFN) systems, and transcription activator-like effector-based nuclease (TALEN) systems.

Exemplary aspects of the CRISPR-Cas system are described in, e.g., U.S. Pat. No. 9,023,649, issued May 5, 2015; U.S. Pat. No. 9,074,199, issued Jul. 7, 2015; and U.S. Pat. No. 8,697,359, issued Apr. 15, 2014 the entire contents of each of which are incorporated herein by reference.

With their highly flexible but specific targeting, CRISPR-Cas systems can be manipulated and redirected to become powerful tools for genome editing. CRISPR-Cas technology permits targeted gene cleavage and gene editing in a variety of eukaryotic cells, and editing can be directed to virtually any genomic locus. Exemplary CRISPR Cas genes include Cas1, Cas2, Cas3′, Cas3″, Cas4, Cas5, Cas6, Cas6e (formerly referred to as CasE, Cse3), Cas6f (i.e., Csy4), Cas7, Cas8a1, Cas8a2, Cas8b, Cas8c, Cas9, Cas10, Cas10d, Csy1, Csy2, CPf1, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, and Csf4. These enzymes are known; for example, the amino acid sequence of Streptococcus pyogenes Cas9 protein may be found in the SwissProt database under accession number Q99ZW2.

Other non-limiting examples of approaches for gene editing include the use of zinc finger nucleases, which are artificial restriction enzymes generated by fusing a zinc finger DNA-binding domain to a DNA-cleavage domain. A zinc finger nuclease is a site-specific endonuclease designed to bind and cleave DNA at specific positions. There are two protein domains. The first domain is the DNA binding domain, which consists of eukaryotic transcription factors and contain the zinc finger. The second domain is the nuclease domain, which consists of the FokI restriction enzyme and is responsible for the catalytic cleavage of DNA. The DNA-binding domains of individual ZFNs typically contain between three and six individual zinc finger repeats and can each recognize between 9 and 18 basepairs. If the zinc finger domains are perfectly specific for their intended target site then even a pair of 3-finger ZFNs that recognize a total of 18 basepairs can, in theory, target a single locus in a mammalian genome. Various strategies have been developed to engineer Cys2His2 zinc fingers to bind desired sequences. These include both “modular assembly” and selection strategies that employ either phage display or cellular selection systems. The most straightforward method to generate new zinc-finger arrays is to combine smaller zinc-finger “modules” of known specificity. The most common modular assembly process involves combining three separate zinc fingers that can each recognize a 3 basepair DNA sequence to generate a 3-finger array that can recognize a 9 basepair target site. Other procedures can utilize either 1-finger or 2-finger modules to generate zinc-finger arrays with six or more individual zinc fingers. Numerous selection methods have been used to generate zinc-finger arrays capable of targeting desired sequences. Initial selection efforts utilized phage display to select proteins that bound a given DNA target from a large pool of partially randomized zinc-finger arrays. More recent efforts have utilized yeast one-hybrid systems, bacterial one-hybrid and two-hybrid systems, and mammalian cells. The non-specific cleavage domain from the type IIs restriction endonuclease FokI is typically used as the cleavage domain in ZFNs. This cleavage domain must dimerize in order to cleave DNA and thus a pair of ZFNs are required to target non-palindromic DNA sites. Standard ZFNs fuse the cleavage domain to the C-terminus of each zinc finger domain. In order to allow the two cleavage domains to dimerize and cleave DNA, the two individual ZFNs must bind opposite strands of DNA with their C-termini a certain distance apart. The most commonly used linker sequences between the zinc finger domain and the cleavage domain requires the 5′ edge of each binding site to be separated by 5 to 7 bp.

TALENs are restriction enzymes that can be engineered to cut specific sequences of DNA. They are made by fusing a TAL effector DNA-binding domain to a DNA cleavage domain. Transcription activator-like effectors (TALEs) can be engineered to bind practically any desired DNA sequence, so when combined with a nuclease, DNA can be cut at specific locations. The restriction enzymes can be introduced into cells, for use in gene editing or for genome editing in situ. Alongside zinc finger nucleases and CRISPR/Cas9, TALEN is a prominent tool in the field of genome editing.

### Pharmaceutical Formulations and Delivery

Dosages, formulations, dosage volumes, regimens, and methods for administering a vector may vary. Thus, minimum and maximum effective dosages vary depending on the method of administration.

“Administering” a vector described herein can be effected or performed using any of the various methods and delivery systems known to those skilled in the art. The administering can be, for example, intravenous, oral, ocular (e.g., subconjunctival, intravitreal, retrobulbar, or intracameral), intramuscular, intravascular, intra-arterial, intracoronary, intramyocardial, intraperitoneal, subcutaneous, inhaled, or intrathecal. Other non-limiting examples include topical administration, or coating of a device to be placed within the subject. In embodiments, administration is effected by injection or via a catheter.

As used herein, “effective” when referring to an amount of a vector refers to the quantity of the vector that is sufficient to yield a desired therapeutic response without undue adverse side effects (such as toxicity, irritation, or allergic response) commensurate with a reasonable benefit/risk ratio when used in the manner of this disclosure.

As used herein, “pharmaceutically acceptable” carrier or excipient refers to a carrier or excipient that is suitable for use with humans and/or animals without undue adverse side effects (such as toxicity, irritation, and allergic response) commensurate with a reasonable benefit/risk ratio. It can be, e.g., a pharmaceutically acceptable solvent, suspending agent or vehicle, for delivering the instant compounds to the subject.

In various embodiments, a composition comprising a vector may be administered only once or multiple times. For example, a vector may be administered using a method disclosed herein at least about once, twice, three times, four times, five times, six times, or seven times per day, week, month, or year. In some embodiments, a composition comprising a vector is administered once per month. In certain embodiments, the composition is administered once per month via intravitreal injection. In various embodiments, such as embodiments involving eye drops, a composition is self-administered. In some embodiments, a viral vector is administered once. In certain embodiments, a non-viral vector (such as a plasmid) is administered more than once, e.g., periodically, or at least about 2, 3, 4, 5, 6, 7, 8, 9, or 10 times.

For the treatment of an ocular disorder, a vector may be administered locally, e.g., as a topical eye drop, peri-ocular injection (e.g., sub-tenon), intraocular injection, intravitreal injection, retrobulbar injection, intraretinal injection, subretinal injection, subconjunctival injection, or using iontophoresis, or peri-ocular devices which can actively or passively deliver drug.

Sustained release of vector (especially in the case of non-viral vectors such as plasmids) may be achieved by the use of technologies such as implants (e.g., solid implants) (which may or may not be bio-degradable) or bio-degradable polymeric matrices (e.g., micro-particles). These may be administered, e.g., peri-ocularly or intravitreally.

Pharmaceutical formulations adapted for topical administration may be formulated as aqueous solutions, ointments, creams, suspensions, lotions, powders, solutions, pastes, gels, sprays, aerosols, liposomes, microcapsules, microspheres, or oils.

The present subject matter provides compositions comprising a vector and a carrier or excipient suitable for administration to ocular tissue. Such carriers and excipients are suitable for administration to ocular tissue (e.g., sclera, lens, iris, cornea, uvea, retina, macula, or vitreous tissue) without undue adverse side effects (such as toxicity, irritation, and allergic response) commensurate with a reasonable benefit/risk ratio.

Pharmaceutical formulations adapted for topical administrations to the eye include eye drops wherein a vector is dissolved or suspended in a suitable carrier, especially an aqueous solvent. Formulations to be administered to the eye will have ophthalmically compatible pH and osmolality. The term “ophthalmically acceptable vehicle” means a pharmaceutical composition having physical properties (e.g., pH and/or osmolality) that are physiologically compatible with ophthalmic tissues.

In some embodiments, an ophthalmic composition of the present invention is formulated as sterile aqueous solutions having an osmolality of from about 200 to about 400 milliosmoles/kilogram water (“mOsm/kg”) and a physiologically compatible pH. The osmolality of the solutions may be adjusted by means of conventional agents, such as inorganic salts (e.g., NaCl), organic salts (e.g., sodium citrate), polyhydric alcohols (e.g., propylene glycol or sorbitol) or combinations thereof.

In various embodiments, the ophthalmic formulations may be in the form of liquid, solid or semisolid dosage form. The ophthalmic formulations may comprise, depending on the final dosage form, suitable ophthalmically acceptable excipients. In some embodiments, the ophthalmic formulations are formulated to maintain a physiologically tolerable pH range. In certain embodiments, the pH range of the ophthalmic formulation is in the range of from about 5 to about 9. In some embodiments, pH range of the ophthalmic formulation is in the range of from about 6 to about 8, or is about 6.5, about 7, or about 7.5.

In some embodiments, the composition is in the form of an aqueous solution, such as one that can be presented in the form of eye drops. By means of a suitable dispenser, a desired dosage of the active agent can be metered by administration of a known number of drops into the eye, such as by one, two, three, four, or five drops.

One or more ophthalmically acceptable pH adjusting agents and/or buffering agents can be included in a composition, including acids such as acetic, boric, citric, lactic, phosphoric, and hydrochloric acids; bases such as sodium hydroxide, sodium phosphate, sodium borate, sodium citrate, sodium acetate, and sodium lactate; and buffers such as citrate/dextrose, sodium bicarbonate, and ammonium chloride. Such acids, bases, and buffers can be included in an amount required to maintain pH of the composition in an ophthalmically acceptable range. One or more ophthalmically acceptable salts can be included in the composition in an amount sufficient to bring osmolality of the composition into an ophthalmically acceptable range. Such salts include those having sodium, potassium, or ammonium cations and chloride, citrate, ascorbate, borate, phosphate, bicarbonate, sulfate, thiosulfate, or bisulfite anions.

Pharmaceutical compositions for ocular delivery also include in situ gellable aqueous composition. Such a composition comprises a gelling agent in a concentration effective to promote gelling upon contact with the eye or with lacrimal fluid. Suitable gelling agents include but are not limited to thermosetting polymers. The term “in situ gellable” as used herein includes not only liquids of low viscosity that form gels upon contact with the eye or with lacrimal fluid, but also includes more viscous liquids such as semi-fluid and thixotropic gels that exhibit substantially increased viscosity or gel stiffness upon administration to the eye. See, for example, Ludwig, Adv. Drug Deliv. Rev. 3; 57:1595-639 (2005), the entire content of which is incorporated herein by reference.

Biocompatible implants for placement in the eye have been disclosed in a number of patents, such as U.S. Pat. Nos. 4,521,210; 4,853,224; 4,997,652; 5,164,188; 5,443,505; 5,501,856; 5,766,242; 5,824,072; 5,869,079; 6,074,661; 6,331,313; 6,369,116; 6,699,493; and 8,293,210, the entire contents of each of which are incorporated herein by reference.

The implants may be monolithic, i.e. having the vector homogenously distributed through the polymeric matrix, or encapsulated, where a reservoir of active agent is encapsulated by the polymeric matrix. Due to ease of manufacture, monolithic implants are usually preferred over encapsulated forms. However, the greater control afforded by the encapsulated, reservoir-type implant may be of benefit in some circumstances, where the therapeutic level of the drug falls within a narrow window. In addition, the therapeutic component, including a vector, may be distributed in a non-homogenous pattern in the matrix. For example, the implant may include a portion that has a greater concentration of a vector relative to a second portion of the implant.

The intraocular implants disclosed herein may have a size of between about 5 μm and about 2 mm, or between about 10 μm and about 1 mm for administration with a needle, greater than 1 mm, or greater than 2 mm, such as 3 mm or up to 10 mm, for administration by surgical implantation. The vitreous chamber in humans is able to accommodate relatively large implants of varying geometries, having lengths of, for example, 1 to 10 mm. The implant may be a cylindrical pellet (e.g., rod) with dimensions of about 2 mm×0.75 mm diameter. The implant may be a cylindrical pellet with a length of about 7 mm to about 10 mm, and a diameter of about 0.75 mm to about 1.5 mm.

The implants may also be at least somewhat flexible so as to facilitate both insertion of the implant in the eye, such as in the vitreous, and accommodation of the implant. The total weight of the implant is usually about 250-5000 μg, more preferably about 500-1000 μg. For example, an implant may be about 500 μg, or about 1000 μg. For non-human subject, the dimensions and total weight of the implant(s) may be larger or smaller, depending on the type of subject. For example, humans have a vitreous volume of approximately 3.8 ml, compared with approximately 30 ml for horses, and approximately 60-100 ml for elephants. An implant sized for use in a human may be scaled up or down accordingly for other animals, for example, about 8 times larger for an implant for a horse, or about, for example, 26 times larger for an implant for an elephant.

Implants can be prepared where the center may be of one material and the surface may have one or more layers of the same or a different composition, where the layers may be cross-linked, or of a different molecular weight, different density or porosity, or the like. For example, where it is desirable to quickly release an initial bolus of vector, the center may be a polylactate coated with a polylactate-polyglycolate copolymer, so as to enhance the rate of initial degradation. Alternatively, the center may be polyvinyl alcohol coated with polylactate, so that upon degradation of the polylactate exterior the center would dissolve and be rapidly washed out of the eye.

The implants may be of any geometry including fibers, sheets, films, microspheres, spheres, circular discs, plaques, and the like. The upper limit for the implant size will be determined by factors such as toleration for the implant, size limitations on insertion, ease of handling, etc. Where sheets or films are employed, the sheets or films will be in the range of at least about 0.5 mm×0.5 mm, usually about 3-10 mm×5-10 mm with a thickness of about 0.1-1.0 mm for ease of handling. Where fibers are employed, the fiber diameter will generally be in the range of about 0.05 to 3 mm and the fiber length will generally be in the range of about 0.5-10 mm. Spheres may be in the range of 0.5 μm to 4 mm in diameter, with comparable volumes for other shaped particles.

The size and form of the implant can also be used to control the rate of release, period of treatment, and drug concentration at the site of implantation. Larger implants will deliver a proportionately larger dose, but depending on the surface to mass ratio, may have a slower release rate. The particular size and geometry of the implant are chosen to suit the site of implantation.

Microspheres for ocular delivery are described, for example, in U.S. Pat. Nos. 5,837,226; 5,731,005; 5,641,750; 7,354,574; and U.S. Pub. No. 2008-0131484, the entire contents of each of which are incorporated herein by reference.

### General Definitions

Unless specifically defined otherwise, all technical and scientific terms used herein shall be taken to have the same meaning as commonly understood by one of ordinary skill in the art (e.g., in cell culture, molecular genetics, and biochemistry).

As used herein, the term “about” in the context of a numerical value or range means ±10% of the numerical value or range recited or claimed, unless the context requires a more limited range.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” In addition, use of the term “based on,” above and in the claims is intended to mean, “based at least in part on,” such that an unrecited feature or element is also permissible

It is understood that where a parameter range is provided, all integers within that range, and tenths thereof, are also provided by the invention. For example, “0.2-5 mg” is a disclosure of 0.2 mg, 0.3 mg, 0.4 mg, 0.5 mg, 0.6 mg etc. up to and including 5.0 mg.

A small molecule is a compound that is less than 2000 daltons in mass. The molecular mass of the small molecule is preferably less than 1000 daltons, more preferably less than 600 daltons, e.g., the compound is less than 500 daltons, 400 daltons, 300 daltons, 200 daltons, or 100 daltons.

As used herein, an “isolated” or “purified” nucleic acid molecule, polynucleotide, polypeptide, or protein, is substantially free of other cellular material, or culture medium when produced by recombinant techniques, or chemical precursors or other chemicals when chemically synthesized. Purified compounds are at least 60% by weight (dry weight) the compound of interest. Preferably, the preparation is at least 75%, more preferably at least 90%, and most preferably at least 99%, by weight the compound of interest. For example, a purified compound is one that is at least 90%, 91%, 92%, 93%, 94%, 95%, 98%, 99%, or 100% (w/w) of the desired compound by weight. Purity is measured by any appropriate standard method, for example, by column chromatography, thin layer chromatography, or high-performance liquid chromatography (HPLC) analysis. A purified or isolated polynucleotide (RNA or DNA) is free of the genes or sequences that flank it in its naturally-occurring state. Purified also defines a degree of sterility that is safe for administration to a human subject, e.g., lacking infectious or toxic agents.

Similarly, by “substantially pure” is meant a nucleotide or polypeptide that has been separated from the components that naturally accompany it. Typically, the nucleotides and polypeptides are substantially pure when they are at least 60%, 70%, 80%, 90%, 95%, or even 99%, by weight, free from the proteins and naturally-occurring organic molecules with they are naturally associated.

As used herein, the term “operably linked” is used to describe the connection between regulatory elements and a gene or its coding region, and the connection between a regulatory element (for example, an operator, a transcription factor binding sequence, or a promoter) and another regulator element. Typically, gene expression is placed under the control of one or more regulatory elements, for example, without limitation, constitutive or inducible promoters, tissue-specific regulatory elements, and enhancers. A gene or coding region is said to be “operably linked to” or “operatively linked to” or “operably associated with” the regulatory elements, meaning that the gene or coding region is controlled or influenced by the regulatory element. For example, a promoter is operably linked to a coding sequence if the promoter effects transcription or expression of the coding sequence. As another example, a transcription factor binding sequence is operably linked to a promoter if the transcription factor binding sequence functions as a regulator (e.g., acts as an on/off switch) for the transcription driven by the promoter.

The terms “coding region” and “coding sequence” as used herein refers to a continuous linear arrangement of nucleotides that may be translated into a protein. A full length coding region is translated into a full length protein; that is, a complete protein as would be translated in its natural state absent any post-translational modifications. A full length coding region may also include any leader protein sequence or any other region of the protein that may be excised naturally from the translated protein.

The term “genetic construct,” as used herein, refers to a recombinant nucleic acid that has been generated for the purpose of the expression of a specific nucleotide sequence(s), or that is to be used in the construction of other recombinant nucleotide sequences.

As used herein, “promoter” refers to a DNA region that controls initiation and rate of transcription. In various embodiments, a promoter can contain genetic elements capable of binding regulatory proteins and other molecules, such as RNA polymerase and other transcription factors. Promoter sequences are commonly, but not always, found in the 5′ non-coding region of genes. A promoter can be functional in a variety of tissue types and in several different species, or its function can be restricted to a particular species and/or a particular tissue or cell type. Further, a promoter can be constitutively active, or it can be selectively activated by certain substances (e.g., a tissue-specific factor), under certain conditions (e.g., heat shock), or during certain developmental stages of the organism (e.g., active in fetus, silent in adult). Examples of promoters include, but are not limited to, promoters from bacteria, yeast, plants, viruses, non-mammal animals, and mammals (including primates such as humans). A promoter can be inducible, repressible, and/or constitutive. Inducible promoters initiate increased levels of transcription from DNA under their control in response to some change in culture conditions, such as a change in temperature.

As used herein, a “tissue-specific” or “cell-specific” promoter refers to a promoter that is capable of driving transcription of a gene in a particular tissue (e.g., vascular tissue, lung, liver, breast, or others) or cell (e.g., mural, endothelial, leukocyte, myocyte, tumor cell, or others) while remaining largely “silent” or expressed at relatively low levels in other tissue or cell types. A tissue-specific or cell-specific promoter can be selective for any tissue or cell-type in a subject. Such promoters are known to one of skill in the art and are disclosed herein. Exemplary of tissue-specific or cell-specific promoters are tumor-specific and cell-specific promoters. It is understood, however, that tissue-specific or cell-specific promoters can have a detectable amount of “background” or “base” activity in those tissues or cells where they are silent. Generally, the promoter is active to a greater degree in a predetermined target cell or tissue as compared to other cells or tissues. For example, the promoter may have at least about 2, 3, 4, 5, 6, 7, 8, 9, 10, 20, 30, 40, 50, 60, 70, 80, 90, 100, 200, 300, 400, 500, 600, 700, 800, 900 times or more activity (i.e. ability to express a nucleic acid sequence operatively linked thereto), in a predetermined tissue or cell than in other tissue or cell types. Thus, a tissue-specific or cell-specific promoter that exhibits some low level activity, e.g., at or about 10% or less in another cell type is still considered to be a tissue-specific or cell-specific promoter if its activity is greater than the activity in a predetermined tissue or cell.

As used herein, the term “gene” refers to any and all discrete coding regions of a host genome, or regions that code for a functional RNA only (e.g., tRNA, rRNA, regulatory RNAs such as ribozymes etc.) as well as associated non-coding regions and optionally regulatory regions. In certain embodiments, the term “gene” includes within its scope the open reading frame encoding specific polypeptides, introns, and adjacent 5′ and 3′ non-coding nucleotide sequences involved in the regulation of expression. In this regard, the gene can further contain control signals such as promoters, enhancers, termination and/or polyadenylation signals that are naturally associated with a given gene, or heterologous control signals. The gene sequences can be cDNA or genomic DNA or a fragment thereof. The gene can be introduced into an appropriate vector for extrachromosomal maintenance or for integration into the host genome.

In various embodiments relating to plasmids, the plasmid is replication competent. As used herein, “replication competent” with reference to a plasmid means that a nucleic acid molecule or plasmid contains the minimal components required for autonomous replication. For purposes herein, a nucleic acid molecule is replication competent if it minimally contains an origin of replication that can be initiated upon binding of a cognate or compatible replication initiator. In some embodiments, a nucleic acid molecule is replication competent if it contains a complete replication unit containing both the origin of replication and a sequence coding for expression of a cognate or compatible replication initiator.

In certain embodiments relating to plasmids, the plasmid is replication-deficient. As used herein, “non-replicating” or “replication-deficient” with reference to a nucleic acid molecule or plasmid refers to a nucleic acid molecule that is not capable of autonomous replication. For example, a non-replicating nucleic acid molecule is one that does not contain an origin of replication.

As used herein, “autonomous replication” with reference to a nucleic acid molecule, such as an autonomously replicating plasmid (ARP), refers to a nucleic acid molecule or plasmid that is capable of self-replication that is episomal or extrachromosomal.

As used herein, an origin of replication (origin) refers to a particular sequence of DNA that is required for replication to begin and at which DNA replication is initiated on a plasmid, virus or chromosome. For purposes herein, an origin of replication includes any origin, including any viral origin such as any polyomavirus origin, that can drive episomal replication in eukaryotic cells, such as mammalian cells or human cells. Exemplary of origins include, but are not limited to, origins from SV40, BKV, JC virus, lymphotropic papovavirus, and simian agent 12. An origin of replication also includes any sequence variant that exhibits a difference in its nucleotide sequence (e.g. due to nucleotide substitution or insertion, truncation or deletion or addition of nucleotides), but that is still capable of initiating replication of DNA in a eukaryotic cell. For example, an origin of replication includes any containing 2, 3, 4, 5, 6, 7, 8, 9, 10 or more binding sites for a compatible or cognate replication initiator.

The term “recombinant cell” refers to a cell that has a new combination of nucleic acid segments that are not covalently linked to each other in nature. A new combination of nucleic acid segments can be introduced into an organism using a wide array of nucleic acid manipulation techniques available to those skilled in the art. A recombinant cell can be a single eukaryotic cell, or a single prokaryotic cell, or a mammalian cell. The recombinant cell can harbor a vector that is extragenomic. An extragenomic nucleic acid vector does not insert into the cell's genome. A recombinant cell can further harbor a vector or a portion thereof that is intragenomic. The term intragenomic defines a nucleic acid construct incorporated within the recombinant cell's genome.

The term “sample” as used herein refers to a biological sample obtained for the purpose of evaluation in vitro. In embodiments, the sample may comprise a body fluid. In some embodiments, the body fluid includes, but is not limited to, whole blood, plasma, serum, lymph, breast milk, saliva, mucous, semen, cellular extracts, inflammatory fluids, cerebrospinal fluid, vitreous humor, tears, vitreous, aqueous humor, or urine obtained from the subject. In some aspects, the sample is a composite panel of two or more body fluids. In exemplary aspects, the sample comprises blood or a fraction thereof (e.g., plasma, serum, or a fraction obtained via leukapheresis). In embodiments, the sample is a tissue sample, such as a biopsy.

A “control” sample or value refers to a sample that serves as a reference, usually a known reference, for comparison to a test sample. For example, a test sample can be taken from a test subject, e.g., a subject with a small vessel disease or in need of diagnosis for a small vessel disease, and compared to samples from known conditions, e.g., a subject (or subjects) that does not have the small vessel disease (a negative or normal control), or a subject (or subjects) who does have the small vessel disease (positive control). A control can also represent an average value gathered from a number of tests or results. One of skill in the art will recognize that controls can be designed for assessment of any number of parameters. One of skill in the art will understand which controls are valuable in a given situation and be able to analyze data based on comparisons to control values. Controls are also valuable for determining the significance of data. For example, if values for a given parameter are variable in controls, variation in test samples will not be considered as significant.

The term, “normal amount” with respect to a compound (e.g., a protein or mRNA) refers to a normal amount of the compound in an individual who does not have a SVD or in a healthy or general population. The amount of a compound can be measured in a test sample and compared to the “normal control” level, utilizing techniques such as reference limits, discrimination limits, or risk defining thresholds to define cutoff points and abnormal values (e.g., for a particular SVD or a symptom thereof). The normal control level means the level of one or more compounds or combined compounds typically found in a subject known not suffering from an SVD. Such normal control levels and cutoff points may vary based on whether a compounds is used alone or in a formula combining with other compounds into an index. Alternatively, the normal control level can be a database of compounds patterns from previously tested subjects who did not develop a SVD or a particular symptom thereof (e.g., in the event the SVD develops or a subject already having the SVD is tested) over a clinically relevant time horizon.

The level that is determined may be the same as a control level or a cut off level or a threshold level, or may be increased or decreased relative to a control level or a cut off level or a threshold level. In some aspects, the control subject is a matched control of the same species, gender, ethnicity, age group, smoking status, body mass index (BMI), current therapeutic regimen status, medical history, or a combination thereof, but differs from the subject being diagnosed in that the control does not suffer from the disease (or a symptom thereof) in question or is not at risk for the disease.

Relative to a control level, the level that is determined may an increased level. As used herein, the term “increased” with respect to level (e.g., protein or mRNA level) refers to any % increase above a control level. In various embodiments, the increased level may be at least or about a 5% increase, at least or about a 10% increase, at least or about a 15% increase, at least or about a 20% increase, at least or about a 25% increase, at least or about a 30% increase, at least or about a 35% increase, at least or about a 40% increase, at least or about a 45% increase, at least or about a 50% increase, at least or about a 55% increase, at least or about a 60% increase, at least or about a 65% increase, at least or about a 70% increase, at least or about a 75% increase, at least or about a 80% increase, at least or about a 85% increase, at least or about a 90% increase, at least or about a 95% increase, relative to a control level.

Relative to a control level, the level that is determined may a decreased level. As used herein, the term “decreased” with respect to level (e.g., protein or mRNA level) refers to any % decrease below a control level. In various embodiments, the decreased level may be at least or about a 5% decrease, at least or about a 10% decrease, at least or about a 15% decrease, at least or about a 20% decrease, at least or about a 25% decrease, at least or about a 30% decrease, at least or about a 35% decrease, at least or about a 40% decrease, at least or about a 45% decrease, at least or about a 50% decrease, at least or about a 55% decrease, at least or about a 60% decrease, at least or about a 65% decrease, at least or about a 70% decrease, at least or about a 75% decrease, at least or about a 80% decrease, at least or about a 85% decrease, at least or about a 90% decrease, at least or about a 95% decrease, relative to a control level.

The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

The terms “subject,” “patient,” “individual,” and the like as used herein are not intended to be limiting and can be generally interchanged. An individual described as a “subject,” “patient,” “individual,” and the like does not necessarily have a given disease, but may be merely seeking medical advice. The terms “subject,” “patient,” “individual,” and the like as used herein include all members of the animal kingdom that may suffer from the indicated disorder. In some aspects, the subject is a mammal, and in some aspects, the subject is a human.

As used herein, the singular forms “a,” “an,” and “the” include the plural reference unless the context clearly dictates otherwise. Thus, for example, a reference to “a disease,” “a disease state”, or “a nucleic acid” is a reference to one or more such embodiments, and includes equivalents thereof known to those skilled in the art and so forth.

As used herein, “treating” encompasses, e.g., inhibition, regression, or stasis of the progression of a disorder. For purposes of this disclosure, beneficial or desired clinical results include, but are not limited to, alleviation or amelioration of any symptom or symptoms, diminishment of extent of disease, stabilized (i.e., not worsening) state of disease, preventing spread of disease, delay or slowing of disease progression, amelioration or palliation of the disease state, and recovery (whether partial or total), whether detectable or undetectable. As used herein, “inhibition” of disease progression or a disease complication in a subject means preventing or reducing the disease progression and/or disease complication in the subject.

As used herein, a “symptom” associated with a disorder includes any clinical or laboratory manifestation associated with the disorder, and is not limited to what the subject can feel or observe.

Embodiments and examples are provided below to facilitate a more complete understanding of the invention. The following embodiments and examples illustrate the exemplary modes of making and practicing the invention. However, the scope of the invention is not limited to specific embodiments disclosed in these embodiments and examples, which are for purposes of illustration only, since alternative methods can be utilized to obtain similar results.

### EMBODIMENTS

Embodiments include Embodiments P1 to P47 following.
Embodiment P1. A method for treating or preventing a small vessel disease (SVD) in a subject, comprising genetically modifying the subject to increase Neurogenic Locus NOTCH Homolog Protein 3 (NOTCH3) expression or activity in the subject.
Embodiment P2. The method of Embodiment P1, wherein genetically modifying the subject comprises replacing a mutant NOTCH3 gene in the subject.
Embodiment P3. The method of Embodiment P2, wherein genetically modifying the subject comprises replacing the mutant NOTCH3 gene, or a mutated portion thereof, with a NOTCH3 gene or a corresponding portion of a NOTCH3 gene that does not comprise the mutation.
Embodiment P4. The method of Embodiment P1, wherein genetically modifying the subject comprises expressing an exogenous NOTCH3 gene in the subject.
Embodiment P5. The method of Embodiment P4, wherein the exogenous NOTCH3 gene is part of a genetic construct.
Embodiment P6. The method of Embodiment P5, wherein the genetic construct comprises a coding sequence that encodes NOTCH3 and a promoter, wherein the coding sequence is operably linked to the promoter.
Embodiment P7. The method of Embodiment P6, wherein genetically modifying the subject comprises administering a non-viral vector that comprises the genetic construct to the subject.
Embodiment P8. The method of Embodiment P7, wherein the non-viral vector comprises a plasmid.
Embodiment P9. The method of Embodiment P8, wherein the plasmid is administered to the subject in a liposome.
Embodiment P10. The method of Embodiment P6, wherein genetically modifying the subject comprises administering a viral vector that comprises the genetic construct to the subject.
Embodiment P11. The method of Embodiment P10, wherein the viral vector comprises a retroviral vector, a adeno-associated viral vector, or a poxvirus vector.
Embodiment P12. The method of Embodiment P6, wherein the promoter is constitutively active in a mammalian cell.
Embodiment P13. The method of Embodiment P12, wherein the promoter is specifically active in a mural cell or an endothelial cell.
Embodiment P14. The method of Embodiment P13, wherein the promoter is specifically active in a mural cell, and the mural cell is a pericyte or a vascular smooth muscle cell.
Embodiment P15. The method Embodiment P14, wherein the promoter comprises a desmin promoter, an alpha-smooth muscle actin (α-SMA) promoter, a SM22 promoter, a NOTCH3 promoter, or a platelet-derived growth factor receptor beta gene (PDGFRβ).
Embodiment P16. The method of Embodiment P13, wherein the promoter is specifically active in an endothelial cell.
Embodiment P17. The method of Embodiment P16, wherein the promoter comprises a Tie2, Fli-1, vascular endothelial-cadherin (VE-cadherin), endoglin, Flt-1, or intercellular adhesion molecule 2 promoter (ICAM-2) promoter.
Embodiment P18. The method of Embodiment P1, wherein genetically modifying the subject comprises administering a genetically modified stem cell, mesenchymal stem cell, induced pluripotent stem cell (iPSC), iPSC-derived pericytes, or iPSC-derived smooth muscle cell to the subject.
Embodiment P19. The method of Embodiment P18, wherein the stem cell, the mesenchymal stem cell or the iPSC is derived from the subject.
Embodiment P20. The method of Embodiment P19, wherein the stem cell or the iPSC has been genetically modified to revert a mutation in a NOTCH3 gene or to express an exogenous NOTCH3 gene.
Embodiment P21. The method of any one of Embodiments P1 to P20, wherein the SVD comprises cerebral SVD.
Embodiment P22. The method of any one of Embodiments P1 to P20, wherein the SVD comprises cerebral autosomal-dominant arteriopathy with subcortical infarcts and leukoencephalopathy (CADASIL).
Embodiment P23. The method of any one of Embodiments P1 to P20, wherein the SVD comprises a NOTCH3 loss-of-function associated SVD.
Embodiment P24. The method of any one of Embodiments P1 to P20, wherein the SVD comprises cerebral autosomal recessive arteriopathy with subcortical infarcts and leukoencephalopathy (CARASIL).
Embodiment P25. The method of any one of Embodiments P1 to P20, wherein the SVD comprises diabetic retinopathy.
Embodiment P26. The method of any one of Embodiments P1 to P20, wherein the SVD comprises age-related macular degeneration (AMD), nephropathy, microangiopathy, heart failure, Alagille syndrome, familial tetralogy of Fallot, patent ductus arteriosus, a cerebral cavernous malformation, or pulmonary arterial hypertension.
Embodiment P27. The method of any one of Embodiments P1 to P26, wherein the subject has at least 1, 2, 3, or 4 grandparents, parents, aunts, uncles, cousins, or siblings who comprise the SVD.
Embodiment P28. The method of any one of Embodiments P1 to P27, wherein the subject comprises diabetes.
Embodiment P29. The method of Embodiment P28, wherein the diabetes is type 1 diabetes or type 2 diabetes.
Embodiment P30. The method of any one of Embodiments P1 to P29, wherein the subject is at least about 80 years old.
Embodiment P31. The method of any one of Embodiments P1 to P30, wherein a test sample obtained from the subject comprises a level of NOTCH3 protein or mRNA that is different than a normal control.
Embodiment P32. The method of Embodiment P31, wherein the test sample comprises a level of NOTCH3 protein or mRNA that is at least about 5%, 10%, 15%, 20%, 25%, 30%, 40%, 45%, 50%, 55%, 60%, 65%, 70%, 75%, 80%, 90%, 95%, 99%, 100%, 5-50%, 50-75%, or 75-100% lower in the test sample compared to a normal control.
Embodiment P33. The method of Embodiment P31, wherein the test sample comprises a level of NOTCH3 activity that is at least about 5%, 10%, 15%, 20%, 25%, 30%, 40%, 45%, 50%, 55%, 60%, 65%, 70%, 75%, 80%, 90%, 95%, 99%, 100%, 5-50%, 50-75%, or 75-100% lower in the test sample compared to a normal control.
Embodiment P34. The method of any one of Embodiments P31-P33, wherein the test sample comprises blood, serum, or plasma.
Embodiment P35. The method of any one of Embodiments P31-P33, wherein the test sample comprises saliva, tears, vitreous, cerebrospinal fluid, sweat, cerebrospinal fluid, or urine.
Embodiment P36. The method of any one of Embodiments P31-P35, wherein a test sample obtained from the subject comprises a level of collagen18α1, endostatin, NOTCH3, N3ECD, insulin-like growth factor binding protein 1 (IGFBP-1), and/or High-Temperature Requirement A Serine Peptidase 1 (HTRA1) protein or mRNA that is different than a normal control.
Embodiment P37. The method of any one of Embodiments P31-P36, wherein the test sample comprises a level of collagen18α1, endostatin, IGFBP-1, and/or HTRA1 protein or mRNA that is at least about 5%, 10%, 15%, 20%, 25%, 30%, 40%, 45%, 50%, 55%, 60%, 65%, 70%, 75%, 80%, 90%, 5-50%, 50-75%, 1-fold, 2-fold, 3-fold, 4-fold, or 5-fold higher in the test sample compared to a normal control.
Embodiment P38. The method of any one of Embodiments P31-P36, wherein the test sample comprises a level of HTRA1 protein or mRNA that is at least about 5%, 10%, 15%, 20%, 25%, 30%, 40%, 45%, 50%, 55%, 60%, 65%, 70%, 75%, 80%, 90%, 95%, 99%, 100%, 5-50%, 50-75%, or 75-100% lower in the test sample compared to a normal control.
Embodiment P39. A composition comprising an effective amount of a vector comprising a genetic construct and an ophthalmically acceptable vehicle, wherein the genetic construct comprises a coding sequence that encodes NOTCH3 and a promoter, wherein the coding sequence is operably linked to the promoter.
Embodiment P40. The composition of Embodiment P39, wherein the vector comprises a plasmid.
Embodiment P41. The composition of Embodiment P39, wherein the vector comprises a viral vector.
Embodiment P42. The composition of any one of Embodiments P39-P41, which is in the form of an aqueous solution comprising an osmolality of about 200 to about 400 milliosmoles/kilogram water.
Embodiment P43. A non-viral vector for treating or preventing a SVD in a subject, wherein the non-viral vector comprises a genetic construct, wherein the genetic construct comprises a coding sequence that encodes NOTCH3 and a promoter, wherein the coding sequence is operably linked to the promoter.
Embodiment P44. The non-viral vector of Embodiment P43, which is a plasmid.
Embodiment P45. A viral vector for treating or preventing a SVD in a subject, wherein the viral vector comprises a genetic construct that comprises a promoter and a coding sequence that encodes NOTCH3, wherein the coding sequence is operably linked to the promoter.
Embodiment P46. The viral vector of Embodiment 45, which is a retroviral vector.
Embodiment P47. Use of a genetic construct in the manufacture of a medicament for treating or preventing a SVD in a subject, wherein the genetic construct comprises a coding sequence that encodes NOTCH3 and a promoter, wherein the coding sequence is operably linked to the promoter.

Additional embodiments include Embodiments 1 to 65 following.

Embodiment 1. A method for treating or preventing a small vessel disease (SVD) in a subject, comprising genetically modifying the subject to increase Neurogenic Locus Notch Homolog Protein 3 (NOTCH3) expression or activity in the subject.
Embodiment 2. The method of Embodiment 1, wherein genetically modifying the subject comprises adding a gene that expresses wild-type NOTCH3 in the subject, wherein the wild-type NOTCH3 comprises the amino acid sequence of SEQ ID NO: 1.
Embodiment 3. The method of Embodiment 1 or 2, wherein genetically modifying the subject comprises administering to the subject a lentivirus particle comprising a transgene that comprises a wild-type NOTCH3 transgene operably linked to a SM22 promoter, wherein administering the lentivirus particle comprises contacting tissue of the subject that is affected by the SVD with the lentivirus particle.
Embodiment 4. The method of Embodiment 1 or 2, wherein genetically modifying the subject comprises contacting a cell with a lentivirus particle comprising a transgene that comprises a wild-type NOTCH3 transgene operably linked to a SM22 promoter, and then administering the cell to the subject.
Embodiment 5. The method of Embodiment 1, wherein genetically modifying the subject comprises replacing a mutant NOTCH3 gene in the subject, wherein a mutant gene encodes a mutant having a C455R mutation compared to SEQ ID NO: 1.
Embodiment 6. The method of Embodiment 1 or 5, wherein genetically modifying the subject comprises replacing a mutant NOTCH3 gene in the subject.
Embodiment 7. The method of Embodiment 1, 5 or 6, wherein genetically modifying the subject comprises replacing the mutant NOTCH3 gene, or a mutated portion thereof, with a NOTCH3 gene or a corresponding portion of a NOTCH3 gene that does not comprise the mutation.
Embodiment 8. The method of any one of Embodiments 1-7, wherein genetically modifying the subject comprises expressing one or more copies of an exogenous NOTCH3 gene in the subject.
Embodiment 9. The method of Embodiment 8, wherein the exogenous NOTCH3 gene is part of a viral or non-viral genetic construct.
Embodiment 10. The method of Embodiment 9, wherein the genetic construct comprises a coding sequence that encodes NOTCH3 and a promoter, wherein the coding sequence is operably linked to the promoter.
Embodiment 11. The method of Embodiment 9 or 10, wherein genetically modifying the subject comprises administering a non-viral vector that comprises the genetic construct to the subject.
Embodiment 12. The method of Embodiment 11, wherein the non-viral vector comprises a plasmid.
Embodiment 13. The method of Embodiment 12, wherein the plasmid is administered to the subject in a liposome.
Embodiment 14. The method of Embodiment 9 or 10, wherein genetically modifying the subject comprises administering a viral vector that comprises the genetic construct to the subject.
Embodiment 15. The method of Embodiment 14, wherein the viral vector comprises a retroviral vector, a adeno-associated viral vector, or a poxvirus vector.
Embodiment 16. The method of any one of Embodiments 10-15, wherein the promoter is constitutively active in a mammalian cell.
Embodiment 17. The method of any one of Embodiments 10-16, wherein the promoter is specifically active in a mural cell or an endothelial cell.
Embodiment 18. The method of Embodiment 17, wherein the promoter is specifically active in a mural cell, and the mural cell is a pericyte or a vascular smooth muscle cell.
Embodiment 19. The method Embodiment 18, wherein the promoter comprises a desmin promoter, an alpha-smooth muscle actin (α-SMA) promoter, a SM22 promoter, a CSPG4 promoter, a SMMHC promoter, a NOTCH3 promoter, or a platelet-derived growth factor receptor beta gene (PDGFRβ).
Embodiment 20. The method of Embodiment 17, wherein the promoter is specifically active in an endothelial cell.
Embodiment 21. The method of Embodiment 17, wherein the promoter comprises a Tie2, Fli-1, vascular endothelial-cadherin (VE-cadherin), endoglin, Flt-1, or intercellular adhesion molecule 2 promoter (ICAM-2) promoter.
Embodiment 22. The method of any one of Embodiments 10-22, wherein the coding sequence is operably linked to a combination of 2 or 3 promoters.
Embodiment 23. The method of any one of Embodiments 1 or 3-22, wherein genetically modifying the subject comprises genetically modifying a cell ex vivo and then administering the cell to the subject.
Embodiment 24. The method of any one of Embodiments 1 or 3-22, wherein genetically modifying the subject comprises administering a genetically modified stem cell, mesenchymal stem cell, induced pluripotent stem cell (iPSC), iPSC-derived pericytes, or iPSC-derived smooth muscle cell to the subject.
Embodiment 25. The method of Embodiment 24, wherein the stem cell, the mesenchymal stem cell or the iPSC is derived from the subject.
Embodiment 26. The method of Embodiment 25, wherein the stem cell or the iPSC has been genetically modified to revert a mutation in a NOTCH3 gene or to express an exogenous NOTCH3 gene.
Embodiment 27. The method of any one of Embodiments 1-26, wherein the SVD comprises cerebral SVD.
Embodiment 28. The method of any one of Embodiments 1-26, wherein the SVD comprises cerebral autosomal-dominant arteriopathy with subcortical infarcts and leukoencephalopathy (CADASIL).
Embodiment 29. The method of any one of Embodiments 1-26, wherein the SVD comprises a NOTCH3 loss-of-function associated SVD.
Embodiment 30. The method of any one of Embodiments 1-26, wherein the SVD comprises cerebral autosomal recessive arteriopathy with subcortical infarcts and leukoencephalopathy (CARASIL).
Embodiment 31. The method of any one of Embodiments 1-26, wherein the SVD comprises diabetic retinopathy.
Embodiment 32. The method of any one of Embodiments 1-26, wherein the SVD comprises a cerebral SVD, CARASIL, CADASIL, age-related macular degeneration (AMD), retinopathy, nephropathy or another SVD of the kidney, microangiopathy, proximal 19p13.12 microdeletion syndrome, myocardial ischemia, heart failure, Alagille syndrome, familial tetralogy of Fallot, patent ductus arteriosus, a cerebral cavernous malformation, or a HTRA1-associated small vessel disease.
Embodiment 33. The method of any one of Embodiments 1-32, wherein the subject has at least 1, 2, 3, or 4 grandparents, parents, aunts, uncles, cousins, or siblings who comprise the SVD.
Embodiment 34. The method of any one of Embodiments 1-33, wherein the subject comprises diabetes.
Embodiment 35. The method of Embodiment 34, wherein the diabetes is type 1 diabetes or type 2 diabetes.
Embodiment 36. The method of any one of Embodiments 1-35, wherein the subject is at least about 80 years old.
Embodiment 37. The method of any one of Embodiments 1-36, wherein the subject comprises a level of NOTCH3 protein or mRNA that is different than a normal control.
Embodiment 38. The method of any one of Embodiments 1-37, wherein the subject comprises a level of NOTCH3 protein or mRNA that is at least about 5%, 10%, 15%, 20%, 25%, 30%, 40%, 45%, 50%, 55%, 60%, 65%, 70%, 75%, 80%, 90%, 95%, 99%, 100%, 5-50%, 50-75%, or 75-100% lower compared to a normal control.
Embodiment 39. The method of any one of Embodiments 1-38, wherein the subject comprises a level of NOTCH3 activity that is at least about 5%, 10%, 15%, 20%, 25%, 30%, 40%, 45%, 50%, 55%, 60%, 65%, 70%, 75%, 80%, 90%, 95%, 99%, 100%, 5-50%, 50-75%, or 75-100% lower compared to a normal control.
Embodiment 40. The method of any one of Embodiments 1-39, wherein the subject comprises a level of collagen18α1 or endostatin protein or mRNA that is at least about 5%, 10%, 15%, 20%, 25%, 30%, 40%, 45%, 50%, 55%, 60%, 65%, 70%, 75%, 80%, 90%, 95%, 99%, 100%, 5-50%, 50-75%, or 75-100% lower compared to a normal control.
Embodiment 41. The method of any one of Embodiments 1-39, wherein the subject comprises a level of NOTCH3 protein bound to collagen18α1 and/or endostatin and/or HTRA1 and/or IGFBP-1 that is at least about 5%, 10%, 15%, 20%, 25%, 30%, 40%, 45%, 50%, 55%, 60%, 65%, 70%, 75%, 80%, 90%, 5-50%, 50-75%, 1-fold, 2-fold, 3-fold, 4-fold, or 5-fold higher compared to a normal control.
Embodiment 42. The method of any one of Embodiments 1-33, wherein the subject comprises a white matter hyperintensity and/or a lacunar stroke as observed by magnetic resonance imaging.
Embodiment 43. The method of any one of Embodiments 1-42, wherein the subject comprises a level of neurofilament light chain (NF-L) protein or activity that is at least about 5%, 10%, 15%, 20%, 25%, 30%, 40%, 45%, 50%, 55%, 60%, 65%, 70%, 75%, 80%, 90%, 5-50%, 50-75%, 1-fold, 2-fold, 3-fold, 4-fold, or 5-fold higher compared to a normal control.
Embodiment 44. The method of Embodiment 40, wherein the subject comprises a level of NOTCH3 activity that is at least about 5%, 10%, 15%, 20%, 25%, 30%, 40%, 45%, 50%, 55%, 60%, 65%, 70%, 75%, 80%, 90%, 95%, 99%, 100%, 5-50%, 50-75%, or 75-100% lower compared to a normal control.
Embodiment 45. The method of any one of Embodiments 37-44, wherein the level is in a test sample obtained from the subject.
Embodiment 46. The method of Embodiment 45, wherein the test sample comprises blood, serum, or plasma.
Embodiment 47. The method of Embodiment 45, wherein the test sample comprises saliva, tears, vitreous, cerebrospinal fluid, sweat, cerebrospinal fluid, or urine.
Embodiment 48. The method of any one of Embodiments 1-53, wherein the subject comprises a level of collagen18α1, endostatin, NOTCH3, N3ECD, insulin-like growth factor binding protein 1 (IGFBP-1), High-Temperature Requirement A Serine Peptidase 1 (HTRA1), MRI and/or NF-L protein or mRNA that is different than a normal control.
Embodiment 49. The method of any one of Embodiments 1-48, wherein the subject comprises a level of collagen18α1, endostatin, IGFBP-1, HTRA1, and/or NF-L protein or mRNA that is at least about 5%, 10%, 15%, 20%, 25%, 30%, 40%, 45%, 50%, 55%, 60%, 65%, 70%, 75%, 80%, 90%, 5-50%, 50-75%, 1-fold, 2-fold, 3-fold, 4-fold, or 5-fold higher compared to a normal control.
Embodiment 50. The method of any one of Embodiments 1-49, wherein the subject comprises a level of HTRA1 protein or mRNA that is at least about 5%, 10%, 15%, 20%, 25%, 30%, 40%, 45%, 50%, 55%, 60%, 65%, 70%, 75%, 80%, 90%, 95%, 99%, 100%, 5-50%, 50-75%, or 75-100% lower compared to a normal control.
Embodiment 51. The method of any one of Embodiments 1-50, wherein the subject comprises a protein-protein complex comprising NOTCH3 bound to collagen18α1/endostatin, HTRA1, IGFBP-1, and/or NOTCH3.
Embodiment 52. The method of Embodiment 51, wherein NOTCH3 is the NOTCH3 extracellular domain (N3ECD).
Embodiment 53. The method of any one of Embodiments 1-52, wherein the subject comprises a N3ECD homodimer.
Embodiment 54. The method of claim 1, wherein the genetic modification is administered as a monotherapy.
Embodiment 55. The method of Embodiment 28, wherein the subject is not administered a thrombolytic agent.
Embodiment 56. The method of any one of Embodiments 1-55, wherein the subject has had a lacunar stroke.
Embodiment 57. The method of any one of Embodiments 1-56, wherein the subject has had a hemorrhagic stroke.
Embodiment 58. The method of any one of Embodiments 1 or 3-55, wherein genetically modifying the subject comprises administering genetically modified histocompatible primary cells to the subject.
Embodiment 59. The method of any one of Embodiments 1 or 3-55, wherein genetically modifying the subject comprises administering genetically modified primary cells or a genetically modified cell line to the subject.
Embodiment 60. The method of Embodiment 58 or 59, wherein the genetically modified cells were obtained from the subject and genetically modified ex vivo before being administered to the subject.
Embodiment 61. The method of Embodiment 58 or 59, wherein the cells were obtained from a donor and genetically modified ex vivo before being administered to the subject.
Embodiment 62. A composition comprising an effective amount of a vector comprising a genetic construct and an ophthalmically acceptable vehicle, wherein the genetic construct comprises a coding sequence that encodes NOTCH3 and a promoter, wherein the coding sequence is operably linked to the promoter.
Embodiment 63. The composition of Embodiment 62, wherein the vector comprises a plasmid.
Embodiment 64. The composition of Embodiment 62, wherein the vector comprises a viral vector.
Embodiment 65. The composition of any one of Embodiments 62-64, which is in the form of an aqueous solution comprising an osmolality of about 200 to about 400 milliosmoles/kilogram water.
Embodiment 66. A non-viral vector for treating or preventing a SVD in a subject, wherein the non-viral vector comprises a genetic construct, wherein the genetic construct comprises a coding sequence that encodes NOTCH3 and a promoter, wherein the coding sequence is operably linked to the promoter.
Embodiment 67. The non-viral vector of Embodiment 66, which is a plasmid.
Embodiment 68. A viral vector for treating or preventing a SVD in a subject, wherein the viral vector comprises a genetic construct that comprises a promoter and a coding sequence that encodes NOTCH3, wherein the coding sequence is operably linked to the promoter.
Embodiment 69. The viral vector of Embodiment 68, which is a retroviral vector.
Embodiment 70. Use of a genetic construct in the manufacture of a medicament for treating or preventing a SVD in a subject, wherein the genetic construct comprises a coding sequence that encodes NOTCH3 and a promoter, wherein the coding sequence is operably linked to the promoter.

### Example 1: Therapeutic Targeting of NOTCH3 Signaling Prevents Mural Cell Loss in Small Vessel Disease

The present disclosure is not limited by any particular scientific theory. However, discussions regarding the potential role of NOTCH3 in SVD are provided to facilitate the understanding of possible mechanisms involved with SVD in various embodiments described herein.

This Example discloses the characterization of a mouse model of SVD in which mural cell coverage in arteries depends upon human NOTCH3 function, a cell signaling mechanism associated with SVD and mural cell pathology in humans. The data presented herein shows that arteriolar degeneration linked to Notch mutations is suppressed by reestabling physiological Notch signaling. Without being bound by any scientific theory, the data herein show Notch loss-of-function (and not Notch toxic gain-of-function or neomorphism) as the relevant mechanism in SVD.

A modality of treatment focused on preventing mural cell loss (a mechanistic cause of SVD) was tested. For that purpose mouse models of NOTCH3 were utilized. NOTCH3 is a gene strongly associated to SVD in humans (Arboleda-Velasquez et al., 2011, Proc Natl Acad Sci USA 108:E128-135; Arboleda-Velasquez et al., 2008, Proc Natl Acad Sci USA 105:4856-4861; Chabriat et al., 2009, Cadasil. Lancet Neurol 8:643-653; Henshall et al., 2015, Arterioscler Thromb Vase Biol 35:409-420; Joutel et al., 1996, Nature 383:707-710). In mammalian cells, Notch receptors at the plasma membrane are heterodimers resulting from an S1 proteolytic cleavage mediated by Furin (Louvi and Artavanis-Tsakonas, 2012, Semin Cell Dev Biol 23:473-480). In the absence of the ligand, a Negative Regulatory Region (NRR) comprising the Lin12-Notch repeats (LNR) and the heterodimerization domain keep the receptor in an autoinhibited configuration stabilized via non-covalent bonds (Xu et al., 2015, Structure 23:1227-1235). Interactions with Notch ligands (DELTA or JAGGED) expose an S2 cleavage site within the NRR to proteolysis by ADAM (A Disintegrin And Metalloproteinase Domain) (Louvi and Artavanis-Tsakonas, 2012, Semin Cell Dev Biol 23:473-480). Presenilin-containing gamma secretase constitutively cuts S-2 cleaved Notch receptors at a transmembrane site (S3) leading to nuclear translocation of the Notch intracellular domain and regulation of transcriptional downstream targets (Kopan, 2012, Cold Spring Harb Perspect Biol 4(10). pii: a011213).

Mutations in NOTCH3 leading to a NOTCH3 receptor with unpaired cysteines in the extracellular domain are a cause of CADASIL, the most common monogenic cause of cerebral SVD (Joutel et al., 1996, Nature 383:707-710). It has been proposed that CADASIL mutations trigger aggregation of the NOTCH3 extracellular domain and aberrant interactions between it and other proteins, leading to neomorphic effects (Arboleda-Velasquez et al., 2005, Hum Mol Genet 14:1631-1639; Chabriat et al., 2009, Lancet Neurol 8:643-653; Joutel et al., 2015, J Cereb Blood Flow Metab 36(1):143-57). CADASIL mutations located in the ligand-binding domain of the NOTCH3 receptor and those that impair plasma membrane localization overtly impair NOTCH3 downstream signaling (Arboleda-Velasquez et al., 2002, Neurology 59:277-279; Arboleda-Velasquez et al., 2011, Proc Natl Acad Sci USA 108:E128-135; Joutel et al., 2004, Am J Hum Genet 74:338-347). A distinct class of NOTCH3 mutations including premature stop codons or frame shift mutations in NOTCH3 are also associated with cerebral SVD; patients with these loss-of-function mutations in NOTCH3 develop symptoms later in life, show incomplete penetrance compared to CADASIL patients, and lack CADASIL's characteristic vascular deposits (e.g. NOTCH3 extracellular domain and granular osmiophilic deposits, GOM)(Dotti et al., 2004, Arch Neurol 61:942-945; Erro et al., 2015, Folia Neuropathol 53:168-171; Moccia et al., 2015, Neurobiol Aging 36:547 e545-511; Pippucci et al., 2015, EMBO Mol Med 7(6):848-58; Rutten et al., 2013, Hum Mutat 34:1486-1489; Yoon et al., 2015, Neurobiol Aging 36:2443 e2441-2447). Consistent with the pathobiology of these human conditions, CADASIL and NOTCH3 knockout mice develop progressive loss of mural cells (Arboleda-Velasquez et al., 2011, Proc Natl Acad Sci USA 108:E128-135; Ghosh et al., 2015, Ann Neurol 78(6):887-900; Henshall et al., 2015, Arterioscler Thromb Vasc Biol 35:409-420; Kofler et al., 2015, Sci Rep 5:16449).

The data herein show that targeting NOTCH3 signaling in mural cells is a useful therapeutic modality in SVD. To examine efficacy of treatment, a roster of morphological biomarkers and retinal vascular leakage analyses were leveraged.

### Results and Discussion

### Mural Cell Coverage in Vessels is Mechanistically Linked to NOTCH3 Signaling

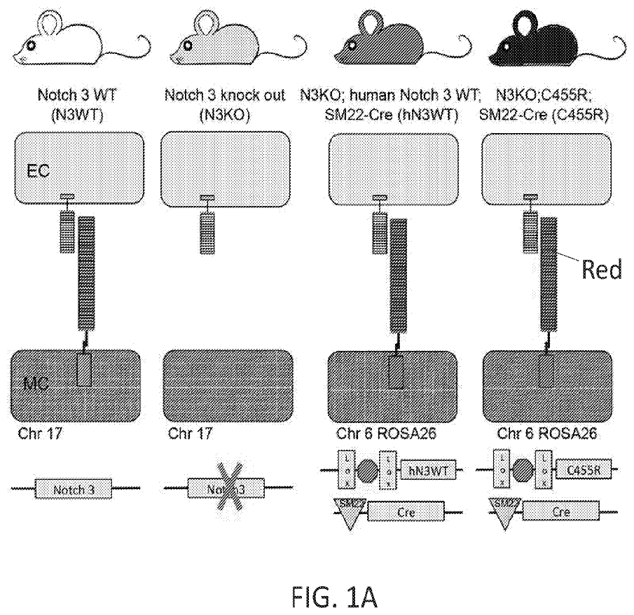

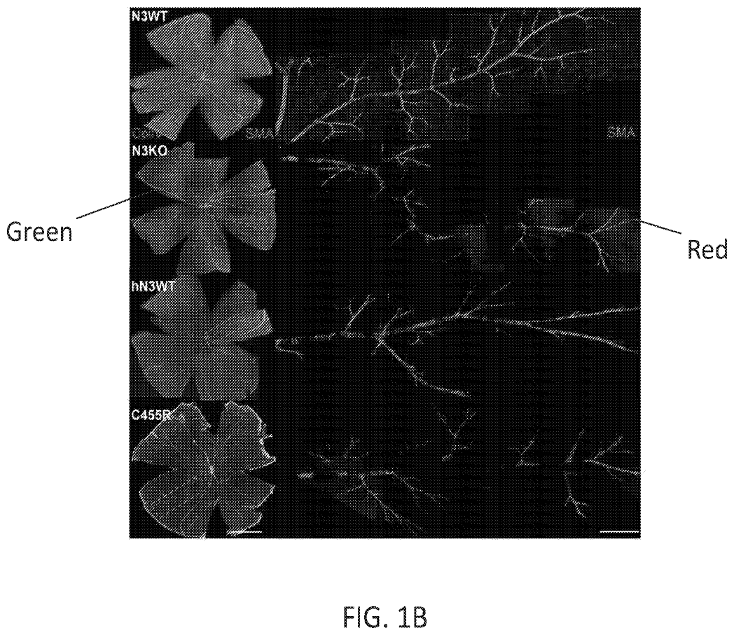

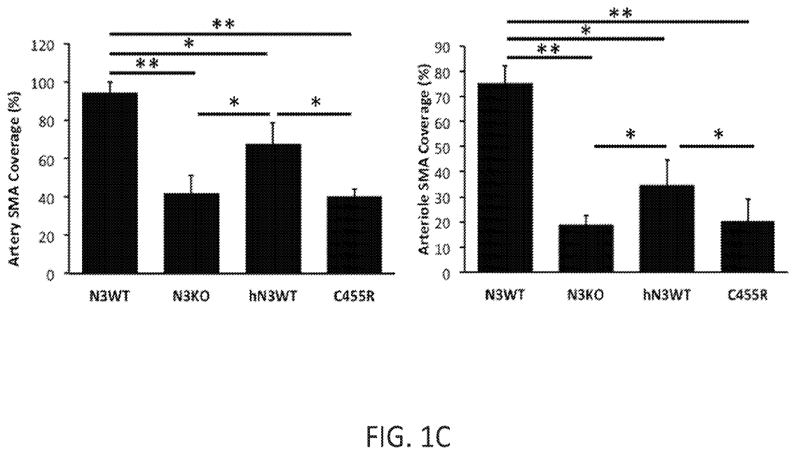

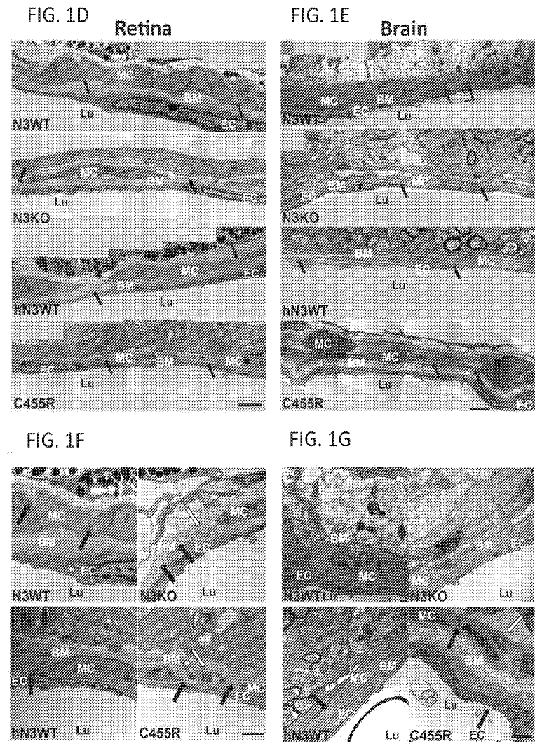

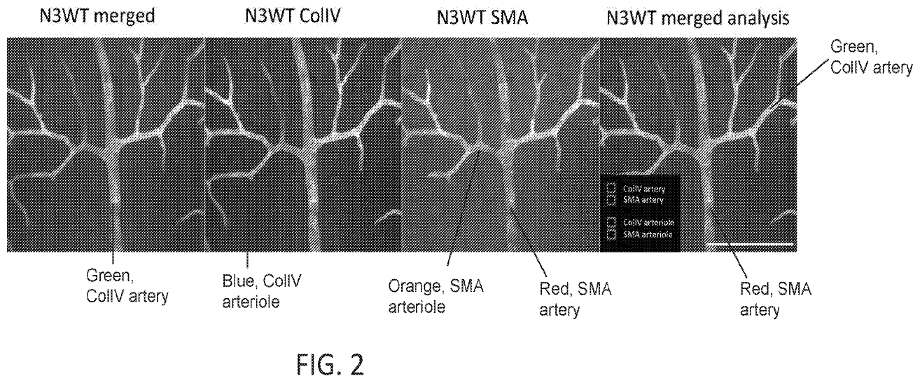

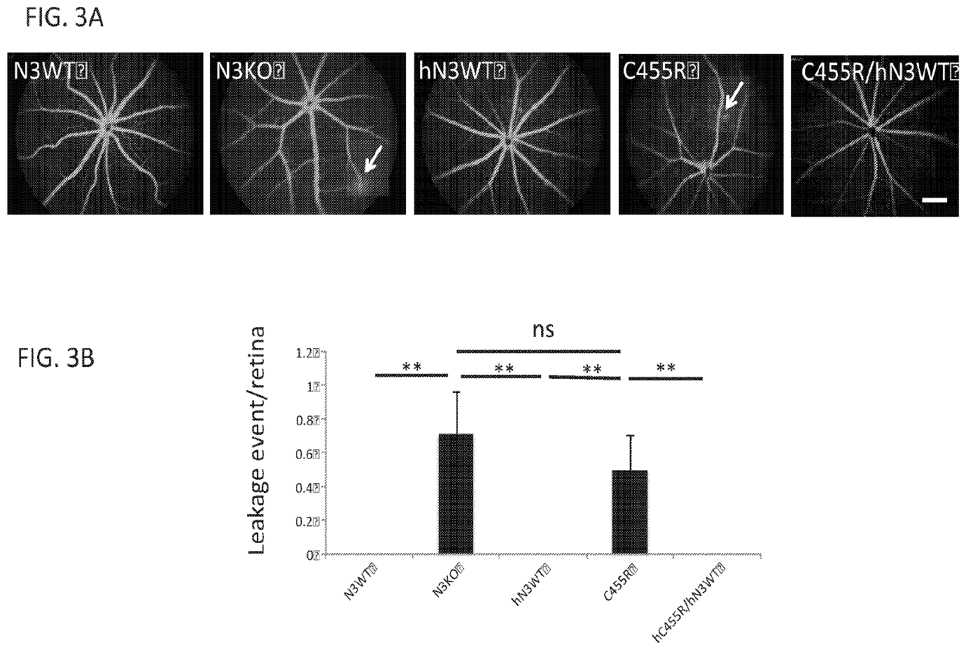

To investigate cell autonomous effects of NOTCH3 signaling in mural cells, mural cell coverage was examined in retinal vessels from NOTCH3 knockout (N3KO) mice and N3KO mice conditionally expressing wild-type (hN3WT) or CADASIL mutant (C455R) alleles of human NOTCH3 in mural cells (FIG. 1A). Morphometric software separated main and branching vessel analyses, quantifying α-smooth muscle actin (SMA) coverage in both (FIG. 4). SMA staining was chosen to detect mural cells because expression of this marker is not impacted by changes in NOTCH3 activity (Arboleda-Velasquez et al., 2014, Invest Ophthalmol Vis Sci 55:5191-5199; Arboleda-Velasquez et al., 2008, Proc Natl Acad Sci USA 105:4856-4861). The retina has a very stereotypic vessel distribution and therefore offers unique advantages for quantitative assessments of changes in vascular structure. Moreover, there is clinical evidence for retinal changes in CADASIL patients (Robinson et al., 2001, Surv Ophthalmol 45:445-448; Rufa et al., 2011, Cerebrovasc Dis 31:77-82).

Absence of NOTCH3 expression was found to dramatically reduce mural cell coverage in retinal arteries and arterioles of six-month old animals (FIG. 1B, C). Furthermore, expression of hN3WT was sufficient to rescue mural cell loss in both large vessels and smaller caliber arteriole branches of N3KO mice (FIG. 1B, C). Indicative of a systemic phenotype, electron microscopy showed large gaps in mural cell coverage in vessels from the brain cortex and the retina in N3KO mice, whereas mural cells were juxtaposed to each other in knockout animals expressing human NOTCH3 in mural cells (FIG. 1D, E). Mural cells undergoing apoptosis were detected within the arterial gaps in N3KO animals (FIG. 1F, G). Altogether, these findings indicate that NOTCH3 signaling is both necessary and sufficient to support mural cell coverage in arteries and is indicative of a cell autonomous effect.

The impact of human NOTCH3 receptor with the C455R mutation was further investigated, because this mutation was identified in a family with early age at onset of CADASIL and can impair ligand-mediated NOTCH3 signaling (Arboleda-Velasquez et al., 2002, Neurology 59:277-279; Arboleda-Velasquez et al., 2011, Proc Natl Acad Sci USA 108:E128-135). The C455R mutant did not rescue nor did it worsen mural cell loss in the N3KO animals (FIG. 1C). It was concluded that the C455R is a loss-of-function mutation and neomorphic effects, previously reported for CADASIL mutations, likely do not contribute to mural cell loss in this SVD model. Additionally, genetic rescue of mural cell loss in N3KO mice by hN3WT supports the notion that patients with SVD due to reduced NOTCH3 signaling may benefit from therapeutic approaches leading to NOTCH3 signaling normalization.

Consistent with the morphological observations, wild-type mice showed no evidence of vascular leakage in the retina using fluorescein angiography whereas NOTCH3 knockout mice and NOTCH3 knockout mice expressing the C455R CADASIL mutation in NOTCH3 showed equally high number of leakage events in the retina. Expression of the human wild-type NOTCH3 was able to significantly reduce the frequency of leakage events in NOTCH3 knockout animals and in NOTCH3 knockout mice expressing the C455R CADASIL mutation.

### Abbreviations

SVD: small vessel disease
NRR: negative regulatory region
SMA: α-smooth muscle actin

### Materials and Methods

Statistical analyses. Pairwise comparisons were assessed using an unpaired two-tailed Student's t-test. One way ANOVA was used to compare more than two experimental groups. Results were considered significant for P<0.05. Analyses were performed and displayed using Prism software (GraphPad).

Animal models. All mouse models used in this study were previously described and were in a C57BL/6 (Arboleda-Velasquez et al., 2011, Proc Natl Acad Sci USA 108:E128-135; Arboleda-Velasquez et al., 2008, Proc Natl Acad Sci USA 105:4856-4861; Mitchell et al., 2001, Nat Genet 28:241-249). Both male and female littermates were included in the study. Briefly, mice are either wild-type (N3WT), lacking endogenous mouse NOTCH3 (N3KO), or express either a wild-type human NOTCH3 transgene (hN3WT, MMRRC:032998 B6; 129 Gt(ROSA)26Sor^{tm1(NOTCH3)Sat}/Mmjax;) or a mutated human NOTCH3 transgene (C455R, MMRRC:033000 129-Gt(ROSA)26Sor^{tm2(NOTCH3*C455R)Sat}/Mmjax) in a N3KO background. Transgenes were inserted into the ROSA26 locus (Soriano, 1999, Nat Genet 21:70-71) and expression of this transgene occurs through Cre-mediated recombination under the smooth muscle cell promoter SM22 (Holtwick et al., 2002, Proc Natl Acad Sci USA 99:7142-7147). The hN3WT and C455R mouse models are available from the Jackson Laboratory under the auspices of the Mutant Mouse Regional Resource Centers program and National Institutes of Health (NIH).

Immunofluorescence. Eyes were harvested and fixed in 4% paraformaldehyde overnight at 4° C. The eyes were then washed three times in phosphate buffered saline (Sigma, D5652-10x1L), at which point retinas were dissected out of each eye and washed as described above. Retinas were then placed in borosilicate glass vials (VWR, 16218-126) and 300 μl of blocking buffer for 6 hrs. Blocking buffer was prepared as previously described (Primo et al., 2016, Brain Res 1644:118-126). Retinas were washed again as above, and double stained with primary antibodies against; goat anti-mouse, smooth muscle actin (Novus, NB300-978) at 1:100 concentration and rabbit anti-mouse collagen IV (Abcam, ab6586) in blocking buffer overnight at 4° C. on a rocker. Retinas were then washed as described above, and immersed for four h at room temperature in secondary antibodies; Donkey Anti-Goat IgG H&L (Cy3 ®) preadsorbed (Abcam, ab6949), Donkey Anti-Rabbit IgG H&L (Alexa Fluor®488) (Abcam, ab150073), all at a 1:100 concentration in blocking buffer. Retinas were then washed as described above and whole mounted on glass slides, (Azer Scientific, EMSC200L), coated with 50% Glycerol in PBS under a rectangular cover slip (Fisher Scientifc, 12-545-F) and sealed with nail polish (REVLON, 8435-76). Entire retinas were imaged at 5×1.25 magnification and three vessels from each retina were imaged at 20×1.25 magnification with an Axioscope 2 Mot Plus (Zeiss).

Electron microscopy (EM): Tissue was fixed in 2.5% glutaraldehyde and 2% paraformaldehyde (PFA) in 0.1 M sodium cacodylate buffer (pH 7.4), rinsed, dehydrated in a series of ethanol dilutions (50-100%), and embedded in epoxy resin (Embed 812; Electron Microscopy Sciences). Ultrathin sections (60 nm) were cut on a Reichert ultramicrotome and collected on Formvar- and carboncoated grids. Samples were stained with 2% uranyl acetate and lead citrate and examined on a Philips Tecnai 12 BioTWIN electron microscope. Images were captured digitally using a CCD camera (Morada; Soft Imaging Systems).

Fluorescein angiography: A Micron III (Phoenix Research) system was used to take fundus photographs in anesthetized mice according to manufacturers instructions. The animals' pupils were dilated using a drop of 1% Tropicamide followed by a drop of 1% cyclopentolate hydrochloride applied on the corneal surface. Eyes were kept moist with ocular lubricant (Genteal). The mice were placed in front of the Fundus camera and pictures of the retina taken. FA was performed after intraperitoneal injection of 0.05 ml of 25% fluorescein sodium (Akron, pharmaceutical grade). Photographs were taken with a preset 20D lens appositioned to the camera lens at regular time (from 1 min to 4 min post IP injection). Fluorescein leakage was noted as diffuse opacity in the vitreous over-time.

### OTHER EMBODIMENTS

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. Genbank and NCBI submissions indicated by accession number cited herein are hereby incorporated by reference. All other published references, documents, manuscripts and scientific literature cited herein are hereby incorporated by reference.

While this invention has been particularly shown and described with references to preferred embodiments thereof, it will be understood by those skilled in the art that various changes in form and details may be made therein without departing from the scope of the invention encompassed by the appended claims.

1. A method for treating or preventing a small vessel disease (SVD) in a subject, comprising genetically modifying the subject to increase wild-type Neurogenic Locus Notch Homolog Protein 3 (NOTCH3) expression or activity in the subject.

2. (canceled)

3. The method of claim 1, wherein genetically modifying the subject comprises A) administering to the subject a lentivirus particle comprising a transgene that comprises a wild-type NOTCH3 transgene operably linked to a SM22 promoter, wherein administering the lentivirus particle comprises contacting tissue of the subject that is affected by the SVD with the lentivirus particle: or B) contacting a cell with a lentivirus particle comprising a transgene that comprises a wild-type NOTCH3 transgene operably linked to a SM22 promoter, and then administering the cell to the subject.

4.-10. (canceled)

11. The method of claim 1, wherein genetically modifying the subject comprises administering a non-viral vector that comprises a viral or non-viral genetic construct comprising an exogenous NOTCH3 gene operably linked to a promotor to the subject.

12.-14. (canceled)

15. The method of claim 11, wherein the viral vector comprises a retroviral vector, a adeno-associated viral vector, or a poxvirus vector.

16.-17. (canceled)

18. The method of claim 11, wherein the promoter is specifically active in pericyte or a vascular smooth muscle cell(s).

19. The method of claim 18, wherein the promoter comprises a desmin promoter, an alpha-smooth muscle actin (α-SMA) promoter, a SM22 promoter, a CSPG4 promoter, a SMMHC promoter, a NOTCH3 promoter, or a platelet-derived growth factor receptor beta gene (PDGFRβ).

20. The method of claim 1, wherein the promoter is specifically active in an endothelial cell.

21. The method of claim 20, wherein the promoter comprises a Tie2, Fli-1, vascular endothelial-cadherin (VE-cadherin), endoglin, Flt-1, or intercellular adhesion molecule 2 promoter (ICAM-2) promoter.

22. The method of claim 19, wherein the promoter comprises a SM22 promoter.

23. The method of claim 1, wherein genetically modifying the subject comprises genetically modifying a cell ex vivo and then administering the cell to the subject.

24. The method of claim 1, wherein genetically modifying the subject comprises administering a genetically modified stem cell, mesenchymal stem cell, induced pluripotent stem cell (iPSC), iPSC-derived pericytes, or iPSC-derived smooth muscle cell to the subject.

25. The method of claim 24, wherein the stem cell, the mesenchymal stem cell or the iPSC is derived from the subject.

26. The method of claim 25, wherein the stem cell or the iPSC has been genetically modified to revert a mutation in a NOTCH3 gene or to express an exogenous NOTCH3 gene.

27. The method of claim 1, wherein the SVD comprises cerebral SVD.

28. The method of claim 1, wherein the SVD comprises cerebral autosomal-dominant arteriopathy with subcortical infarcts and leukoencephalopathy (CADASIL).

29. The method of claim 1, wherein the SVD comprises a NOTCH3 loss-of-function associated SVD.

30. The method of claim 1, wherein the SVD comprises cerebral autosomal recessive arteriopathy with subcortical infarcts and leukoencephalopathy (CARASIL).

31. The method of claim 1, wherein the SVD comprises diabetic retinopathy.

32. The method of claim 1, wherein the SVD comprises a cerebral SVD, CARASIL, CADASIL, age-related macular degeneration (AMD), retinopathy, nephropathy or another SVD of the kidney, microangiopathy, proximal 19p13.12 microdeletion syndrome, myocardial ischemia, heart failure, Alagille syndrome, familial tetralogy of Fallot, patent ductus arteriosus, a cerebral cavernous malformation, or a HTRA1-associated small vessel disease.

33.-61. (canceled)

62. A composition comprising an effective amount of a vector comprising a genetic construct and an ophthalmically acceptable vehicle, wherein the genetic construct comprises a coding sequence that encodes NOTCH3 and a promoter, wherein the coding sequence is operably linked to the promoter.

63.-67. (canceled)

68. A viral vector for treating or preventing a SVD in a subject, wherein the viral vector comprises a genetic construct that comprises a promoter and a coding sequence that encodes NOTCH3, wherein the coding sequence is operably linked to the promoter.

69.-70. (canceled)
